(19) United States
(12) Patent Application Publication
(10) Pub. No.: US 2025/0014683 A1
(43) Pub. Date: Jan. 9, 2025

(54) SYSTEMS AND METHODS FOR POLYMER SEQUENCE PREDICTION

(71) Applicant: ZYMEWORKS BC INC., Vancouver (CA)

(72) Inventors: James Liam McWhirter, Vancouver (CA); Gregory Lakatos, Vancouver (CA); Surjit Bhimarao Dixit, Vancouver (CA); Abhishek Mukhopadhyay, Vancouver (CA); Patrick Farber, Vancouver (CA)

(21) Appl. No.: 18/705,910

(22) PCT Filed: Nov. 1, 2022
(86) PCT No.: PCT/CA2022/051613

§ 371 Date: Apr. 29, 2024

Related U.S. Application Data
(60) Provisional application No. 63/274,403, filed on Nov. 1, 2021.

(51) Int. Cl. G16B 40/20; G16B 15/30; G16H 20/10

(52) U.S. Cl. CPC G16B 40/20; G16B 15/30; G16H 20/10

(57) ABSTRACT

Systems and methods for polymer sequence prediction are provided. Atomic coordinates for at least the main chain atoms of a polypeptide comprising a plurality of residues is obtained and used to encode the residues into residue feature sets. Each residue feature set comprises, for the respective residue and for each neighboring residue within a nearest neighbor cutoff, an indication of secondary structure, a relative solvent accessible surface area of backbone atoms, and cosine and sine values of backbone dihedrals; a C_{α} to C_{α} distance of each neighboring residue; and an orientation and position of each neighboring residue backbone relative to the backbone residue segment of the respective residue. A residue in the plurality of residues is identified, and the corresponding residue feature set is inputted into a neural network comprising at least 500 parameters, thus obtaining a plurality of probabilities, including a probability for each naturally occurring amino acid.

### CROSS REFERENCE TO RELATED PATENT APPLICATIONS

This application claims priority to U.S. Provisional Patent Application No. 63/274,403 entitled “Systems and Methods for Polymer Sequence Prediction,” filed Nov. 1, 2021, which is hereby incorporated by reference.

### TECHNICAL FIELD

The disclosed embodiments relate generally to systems and methods for predicting polymer sequences using neural networks. The disclosed embodiments have wide range of applications in efforts in understanding and improving the physical properties of molecules.

### BACKGROUND

The design of novel proteins is a challenging problem, requiring a search over a huge space of possible structural and chemical changes. Even when complemented with modern experimental techniques and computer-aided design tools, the modeling of proteins such as bispecific antibodies remains challenging and time-consuming. Such engineering efforts often require multiple rounds of rational design, followed by experimental screening of variants or mutations. When trying to conceive of novel protein mutations during rational design and optimization, protein engineers are guided by physicochemical rules which describe interactions limited to pairs and triplets of amino acid residues; however, these rules are incomplete, unable to predict higher-order, coordinated interactions between residues in a large group.

Despite these difficulties, the design process is partially simplified by starting with a known folded protein structure. Given a known folded structure, protein sequence design becomes an inverse-protein folding problem: different combinations of amino acid swaps are applied at different sequence positions to a folded structure, with a fixed or approximately fixed backbone, in the search for sequence mutations that potentially enhance a desired protein characteristic while maintaining, or even enhancing, protein stability. See, e.g., Wernisch et al., “Automated protein design with all atom force-fields by exact and heuristic optimization.” J Mol. Biol. 301, 713-736 (2000); Xiang and Honig, “Extending the Accuracy Limits of Prediction for Side-chain Conformations.” J Mol. Biol. 311, 421-430 (2001); and Simonson et al., “Computational Protein Design: The Proteus Software and Selected Applications.” J Comp. Chem. 34, 2472-2484 (2013). However, despite the significant simplification derived from constraining a protein structure to a known fold, the space of all possible sequences can still be potentially enormous. Thus, armed with physicochemical rules and knowledge gained from previous design rounds, it is still difficult for a protein engineer to determine how amino acid swaps at different residue positions might couple together in order to satisfy one or more physical objectives.

Substantial protein design challenges exist where it would be extremely valuable to have computational, automated methods and workflows, which are able to propose novel mutations previously inconceivable by a traditional physicochemical modeling and simulation based rational design process. The design of functional proteins is usually a multi-objective satisfaction problem with several, often competing objectives, such as, for example, changing the function of a protein while maintaining its stability. See, e.g., Havranek, “Specificity in Computational Protein Design.” J. Biol. Chem. 285, 41, 31095-31099 (2010); Leaver-Fay et al., “A Generic Program for Multistate Protein Design.” PLoS ONE 6, 7, e20937 (2011); and Leaver-Fay et al., “Computationally Designed Bispecific Antibodies Using Negative State Repertoires.” Structure 24, 4, 641-651 (2016).

In particular, to produce bispecific antibodies, the heterodimerization of two different Fc fragments is a requirement for the formation of heavy chain heterodimers. For this antibody Fc engineering problem, the objective is to promote Fc heterodimer formation while suppressing the formation of two corresponding Fc homodimers. However, in rational design processes, achieving this main objective may come at the initial cost of not satisfying the second objective, which is maintaining or enhancing the stability of the Fc heterodimer. The ultimate fulfillment of both these objectives requires several design rounds with mutations involving multiple residue sites and/or sequence positions. See, for instance, Ridgeway et al., “‘Knobs-into-Holes’ engineering of antibody CH3 domains for heavy chain heterodimerization.” Protein Engineering vol. 9 no. 7 pp. 617-621 (1996); Carter, “Bispecific human IgG by design.” J Immuno. Methods 248, 7-15 (2001); Gunasekaran et al., “Enhancing Antibody Fc Heterodimer Formation through Electrostatic Steering Effects.” J Biol. Chem. 285, 25, 19637-19646 (2010); Von Kreudenstein et al., “Improving biophysical properties of a bispecific antibody scaffold to aid developability.” mAbs 5, 5, 646-654 (2013); and Ha et al., “Immunoglobulin Fc Heterodimer Platform Technology: From Design to Applications in Therapeutic Antibodies and Proteins.” Front Immunol. 7, 394 (2016).

As alluded to above, protein design is essentially a combinatorial problem. Consequently, in the absence of expert or prior knowledge about the couplings between the mutation sites, there can be a combinatorial explosion of possible solutions, many of which are incorrect. For example, consider the case in which there are L mutation sites in a region targeted for design and 4 possible choices for amino acid swaps per site. In such a case, there will be 4^{L} (4 to the power of L) possible combinations (e.g., mutations) to test. Prior work on designing a Fc heterodimer demonstrated that successfully satisfying the two objectives of enhanced stability and enhanced binding specificity often required a value of L greater than 7. Moreover, there can be many possible choices of target design regions to screen (i.e., recognizing which regions are the L mutation sites of interest). The large number of potential screening sites increases the complexity of the problem. Further, there can be negative design requirements which constrain the nature of mutations, thereby adding to the complexity of screening and optimization required.

Accordingly, there is a need in the art for methods that can quickly and accurately reduce the number of possible amino acid identities at any given target mutation site to those that are the most probable. In other words, there is a need in the art for computationally automated protein design methods and workflows that can predict the properties of mutation sites in a target design region and generate polymer sequence designs which accurately satisfy one or more physical objectives.

### SUMMARY

The present disclosure addresses the need in the art. Disclosed are systems and methods for predicting polymer sequences by determining probabilities for a plurality of naturally occurring amino acids for a respective residue in a polypeptide, using a neural network. In some implementations, an amino acid identity is determined based on the obtained probabilities, such as by selecting an amino acid identity having a maximum-valued probability and/or by drawing from the plurality of amino acids based on the obtained probabilities. An amino acid for the respective residue is swapped with the determined amino acid, and residue features for residues in the polypeptide are updated based on the swap. In some embodiments, the identity of the respective residue is selected when the updating of the residue features based on the change in amino acid identity satisfies one or more criteria, such as a convergence criterion and/or one or more polypeptide properties (e.g., design objectives).

Advantageously, in some embodiments, the systems and methods disclosed herein provide improved prediction of amino acid identities at target residue sites using a multi-stage neural network model, the neural network model including a first stage comprising parallel branches of sequential convolution layers. Additionally, in some embodiments, the systems and methods disclosed herein provide improved sequence and mutation sampling in order to simultaneously satisfy multiple design objectives. For example, in some such implementations, the systems and methods disclosed herein provide simultaneous tracking of sequence identities (e.g., selection and/or swapping of amino acid identities and determination of polypeptide properties thereof) for a plurality of chemical species (e.g., multiple residue sites and/or sequence positions). In another example, in some such implementations, the systems and methods disclosed herein utilize algorithms (e.g., Gibbs sampling, Metropolis criteria) to guide and control a distribution of candidate polypeptide designs towards enhanced values for a plurality of properties of interest.

In more detail, one aspect of the present disclosure provides a computer system for polymer sequence prediction, the computer system comprising one or more processors, and memory addressable by the one or more processors, the memory storing at least one program for execution by the one or more processors. The at least one program comprises instructions for obtaining a plurality of atomic coordinates for at least the main chain atoms of a polypeptide, where the polypeptide comprises a plurality of residues.

The plurality of atomic coordinates is used to encode each respective residue in the plurality of residues into a corresponding residue feature set in a plurality of residue feature sets, where the corresponding residue feature set includes, for the respective residue and for each respective residue having a C_{α} carbon that is within a nearest neighbor cutoff of the C_{α} carbon of the respective residue (i) an indication of the secondary structure of the respective residue encoded as one of helix, sheet and loop, (ii) a relative solvent accessible surface area of the C_{α}, N, C, and O backbone atoms of the respective residue, and (iii) cosine and sine values of the backbone dihedrals ϕ, ψ and ω of the respective residue. The corresponding residue feature set further includes a C_{α} to C_{α} distance of each neighboring residue having a C_{α} carbon within a threshold distance of the C_{α} carbon of the respective residue, and an orientation and position of a backbone of each neighboring residue relative to the backbone residue segment of the respective residue in a reference frame centered on C_{α} atom of the respective residue.

A respective residue in the plurality of residues is identified, and the residue feature set corresponding to the identified respective residue is inputted into a neural network comprising at least 500 parameters. Thus, a plurality of probabilities is obtained, including a probability for each respective naturally occurring amino acid.

In some embodiments, the at least one program further comprises instructions for selecting, as the identity of the respective residue, the naturally occurring amino acid having the greatest probability in the plurality of probabilities.

In some embodiments, the at least one program further comprises, for each respective residue in at least a subset of the plurality of residues, randomly assigning an amino acid identity to the respective residue prior to the using the plurality of atomic coordinates to encode each respective residue in the plurality of residues into a corresponding residue feature set. For each respective residue in the at least a subset of the plurality of residues, a procedure is performed comprising performing the identifying and the inputting to obtain a corresponding plurality of probabilities for the respective residue, obtaining a respective swap amino acid identity for the respective residue based on a draw from the corresponding plurality of probabilities, and, when the respective swap amino acid identity of the respective residue changes the identity of the respective residue, updating each corresponding residue feature set in the plurality of residue feature sets affected by the change in amino acid identity. In some embodiments, the procedure is repeated until a convergence criterion is satisfied.

In some embodiments, the convergence criterion is a requirement that the identity of none of the amino acid residues in at least the subset of the plurality of residues is changed during the last instance of the procedure performed for each residue in at least the subset of the plurality of residues.

In some embodiments, the obtaining a respective swap amino acid identity for the respective residue based on a draw from the corresponding plurality of conditional probabilities comprises determining a respective difference, E_{final}−Eᵢₙᵢₜᵢₐₗ, between (i) a property of the polypeptide without the respective swap amino acid identity for the respective residue (Eᵢₙᵢₜᵢₐₗ) against (ii) a property of the polypeptide with the respective swap amino acid identity for the respective residue (E_{final}) to determine whether the respective swap amino acid identity for the respective residue improves the property. When the respective difference indicates that the respective swap amino acid identity for the respective residue improves the property of the polypeptide, the identity of the respective residue is changed to the respective swap amino acid identity, and, when the respective difference indicates that the respective swap amino acid identity for the respective residue fails to improve the property of the polypeptide, the identity of the respective residue is conditionally changed to the respective swap amino acid identity based on a function of the respective difference. In some embodiments, the (i) property of the polypeptide without the respective swap amino acid identity for the respective residue (Eᵢₙᵢₜᵢₐₗ) is of the same type as (ii) the property of the polypeptide with the respective swap amino acid identity for the respective residue (E_{final}). Thus, in some embodiments, the difference between the property Eᵢₙᵢₜᵢₐₗ and the property E_{final} is a difference between a metric that is measured with and without the swap amino acid identity.

In some embodiments, the function of the respective difference has the form e^{-(E}^{_{final}}^{-E}^{ᵢₙᵢₜᵢₐₗ}^{)/T}, wherein T is a predetermined user adjusted temperature.

In some embodiments, the property of the polypeptide is a stability of the polypeptide in forming a heterocomplex with a polypeptide of another type. In some embodiments, the polypeptide is an Fc chain of a first type, the polypeptide of another type is an Fc chain of a second type, and the property of the polypeptide is a stability of a heterodimerization of the Fc chain of a first type with the Fc chain of the second type.

In some embodiments, the property of the polypeptide is a composite of (i) a stability of the polypeptide within a heterocomplex with a polypeptide of another type, and (ii) a stability of the polypeptide within the homocomplexes. In some embodiments, the (i) stability of the polypeptide within a heterocomplex with a polypeptide of another type comprises the same type of stability measure as the (ii) stability of the polypeptide within the homocomplexes. In some embodiments, the stabilities of the polypeptide in the homocomplexes are defined using a weighted average of the stability of each homocomplex with the weights bound by [0,1] and sum to 1. In some embodiments, the stability of the polypeptide in the homocomplexes is a non-linear weighted average of the stability of each homocomplex with the weights bound by [0,1] and sum to 1.

In some embodiments, the property of the polypeptide is a composite of (i) a combination of a stability of the polypeptide within a heterocomplex with a polypeptide of another type and a binding specificity or binding affinity of the polypeptide for the polypeptide of another type, and (ii) a combination of a stability of the polypeptide within a homocomplex and a binding specificity or binding affinity of the polypeptide for itself to form the homocomplexes. In some embodiments, the (i) combination of the stability of the polypeptide within a heterocomplex with a polypeptide of another type and the binding specificity or binding affinity of the polypeptide for the polypeptide of another type includes the same type of stability metric and the same type of binding specificity or binding affinity metric as the (ii) combination of a stability of the polypeptide within a homocomplex and a binding specificity or binding affinity of the polypeptide for itself to form the homocomplexes. Thus, in some embodiments, the combination of stability and binding specificity or binding affinity used to characterize the polypeptide within a heterocomplex includes the same types of metrics as the combination of stability and binding specificity or binding affinity used to characterize the polypeptide within a homocomplex. In some embodiments, the stability, binding specificity or binding affinity of the polypeptide in the homocomplexes are defined using a weighted average of the stability, binding specificity or binding affinity of each homocomplex with the weights bound by [0,1] and sum to 1. In some embodiments, the stability, binding specificity or binding affinity of the polypeptide in the homocomplexes is a non-linear weighted average of the stability, binding specificity or binding affinity of each homocomplex with the weights bound by [0,1] and sum to 1.

In some embodiments, the property of the polypeptide is a stability of polypeptide, a pI of polypeptide, a percentage of positively charged residues in the polypeptide, an extinction coefficient of the polypeptide, an instability index of the polypeptide, or an aliphatic index of the polypeptide, or any combination thereof.

In some embodiments, the polypeptide is an antigen-antibody complex.

In some embodiments, the plurality of residues comprises 50 or more residues. In some embodiments, the subset of the plurality of residues is 10 or more, 20 or more, or 30 more residues within the plurality of residues.

In some embodiments, the nearest neighbor cutoff is the K closest residues to the respective residue as determined by C_{α} carbon to C_{α} carbon distance, where K is a positive integer of 10 or greater. In some embodiments, K is between 15 and 25.

In some embodiments, the corresponding residue feature set comprises an encoding of one or more physicochemical property of each side-chain of each residue within the nearest neighbor cutoff of the C_{α} carbon of the respective residue.

In some embodiments, the neural network comprises a first-stage one-dimensional sub-network architecture that feeds into a fully connected neural network having a final node that outputs the probability of each respective naturally occurring amino acid as a twenty element probability vector in which the twenty elements sum to 1.

In some embodiments, the first-stage one-dimensional sub-network architecture comprises a plurality of pairs of convolutional layers, including a first pair of convolutional layers and a second pair of convolutional layers. The first pair of convolutional layers includes a first component convolutional layer and a second component convolutional layer that each receive the residue feature set during the inputting, and the second pair of convolutional layers includes a third component convolutional layer and a fourth component convolutional layer. The first component convolutional layer of the first pair of convolutional layers and the third component convolutional layer of the second pair of convolutional layers each convolve with a first filter dimension, and the second component convolutional layer of the first pair of convolutional layers and the fourth component convolutional layer of the second pair of convolutional layers each convolve with a second filter dimension that is different than the first filter dimension. A concatenated output of the first and second component convolutional layers of the first pair of convolutional layers serves as input to both the third component and fourth component convolutional layers of the second pair of convolutional layers.

In some embodiments, the plurality of pairs of convolutional layers comprises between two and ten pairs of convolutional layers, each respective pair of convolutional layers includes a component convolutional layer that convolves with the first filter dimension, and each respective pair of convolutional layers includes a component convolutional layer that convolves with the second filter dimension. Each respective pair of convolutional layers other than a final pair of convolutional layers in the plurality of pairs of convolutional layers passes a concatenated output of the component convolutional layers of the respective convolutional layer into each component convolutional layer of another pair of convolutional layers in the plurality of convolutional layers.

In some embodiments, the first filter dimension is one and the second filter dimension is two.

In some embodiments, the neural network is characterized by a first convolution filter and a second convolutional filter that are different in size.

In some embodiments, the at least one program further comprises instructions for training the neural network to minimize a cross-entropy loss function across a training dataset of reference protein residue sites labelled by their amino acid designations obtained from a dataset of protein structures.

In some embodiments, the at least one program further comprises instructions for using the probability for each respective naturally occurring amino acid for the respective residue to determine an identity of the respective residue, using the respective residue to update an atomic structure of the polypeptide, and using the updated atomic structure of the polypeptide to determine, in silico, an interaction score between the polypeptide and a composition. In some embodiments, the polypeptide is an enzyme, the composition is being screened in silico to assess an ability to inhibit an activity of the enzyme, and the interaction score is a calculated binding coefficient of the composition to the enzyme. In some embodiments, the protein is a first protein, the composition is a second protein being screened in silico to assess an ability to bind to the first protein in order to inhibit or enhance an activity of the first protein, and the interaction score is a calculated binding coefficient of the second protein to the first protein. In some embodiments, the protein is a first Fc fragment of a first type, the composition is a second Fc fragment of a second type, and the interaction score is a calculated binding coefficient of the second Fc fragment to the first Fc fragment.

Another aspect of the present disclosure provides a non-transitory computer readable storage medium storing one or more computational modules for polymer sequence prediction. The one or more computational modules collectively comprise instructions for obtaining a plurality of atomic coordinates for at least the main chain atoms of a polypeptide, wherein the polypeptide comprises a plurality of residues. The plurality of atomic coordinates is used to encode each respective residue in the plurality of residues into a corresponding residue feature set in a plurality of residue feature sets, where the corresponding residue feature set comprises, for the respective residue and for each respective residue having a C_{α} carbon that is within a nearest neighbor cutoff of the C_{α} carbon of the respective residue (i) an indication of the secondary structure of the respective residue encoded as one of helix, sheet and loop, (ii) a relative solvent accessible surface area of the C_{α}, N, C, and O backbone atoms of the respective residue, and (iii) cosine and sine values of backbone dihedrals ϕ, ψ and ω of the respective residue. The corresponding residue feature set further includes a C_{α} to C_{α} distance of each neighboring residue having a C_{α} carbon within a threshold distance of the C_{α} carbon of the respective residue, and an orientation and position of a backbone of each neighboring residue relative to the backbone residue segment of the respective residue in a reference frame centered on C_{α} atom of the respective residue. A respective residue in the plurality of residues is identified, and the residue feature set corresponding to the identified respective residue is inputted into a neural network comprising at least 500 parameters, Thus, a plurality of probabilities is obtained, including a probability for each respective naturally occurring amino acid.

Another aspect of the present disclosure provides a method for polymer sequence prediction. The method comprises, at a computer system comprising a memory, obtaining a plurality of atomic coordinates for at least the main chain atoms of a polypeptide, where the polypeptide comprises a plurality of residues. The plurality of atomic coordinates is used to encode each respective residue in the plurality of residues into a corresponding residue feature set in a plurality of residue feature sets, where the corresponding residue feature set comprises, for the respective residue and for each respective residue having a C_{α} carbon that is within a nearest neighbor cutoff of the C_{α} carbon of the respective residue, (i) an indication of the secondary structure of the respective residue encoded as one of helix, sheet and loop, (ii) a relative solvent accessible surface area of the C_{α}, N, C, and O backbone atoms of the respective residue, and (iii) cosine and sine values of backbone dihedrals ϕ, ψ and ω of the respective residue. The corresponding residue feature set further includes a C_{α} to C_{α} distance of each neighboring residue having a C_{α} carbon within a threshold distance of the C_{α} carbon of the respective residue, and an orientation and position of a backbone of each neighboring residue relative to the backbone residue segment of the respective residue in a reference frame centered on C_{α} atom of the respective residue. A respective residue in the plurality of residues is identified, and the residue feature set corresponding to the identified respective residue is inputted into a neural network comprising at least 500 parameters. Thus, a plurality of probabilities is obtained, including a probability for each respective naturally occurring amino acid.

### BRIEF DESCRIPTION OF THE DRAWINGS

The embodiments disclosed herein are illustrated by way of example, and not by way of limitation, in the figures of the accompanying drawings. Like reference numerals refer to corresponding parts throughout the drawings.

FIGS. 1A and 1B collectively illustrate a computer system in accordance with some embodiments of the present disclosure.

FIGS. 2A, 2B, 2C, 2D, 2E, 2F, and 2G collectively illustrate an example method for polymer sequence prediction, in which optional steps are indicated by dashed boxes, in accordance with some embodiments of the present disclosure.

FIGS. 3A, 3B, 3C, and 3D collectively illustrate an example neural network workflow and architecture for polymer sequence prediction, in accordance with some embodiments of the present disclosure.

FIGS. 4A, 4B, and 4C collectively illustrate performance metrics obtained using an example neural network for polymer sequence prediction, in accordance with an embodiment of the present disclosure.

FIGS. 5A and 5B illustrate performance metrics obtained using an example neural network for polymer sequence prediction, in accordance with an embodiment of the present disclosure.

FIGS. 6A, 6B, and 6C illustrate amino acid identity predictions for heterodimeric Fc (HetFc) design, in accordance with an embodiment of the present disclosure.

FIGS. 7A, 7B, and 7C illustrate performance metrics obtained using an example neural network for polymer sequence prediction, in accordance with an embodiment of the present disclosure.

FIGS. 8A, 8B, and 8C illustrate performance metrics obtained using an exemplary biased sampling procedure, in accordance with an embodiment of the present disclosure.

FIGS. 9A, 9B, 9C, 9D, and 9E illustrate hierarchical clustering of polymer sequences generated using an exemplary biased sampling procedure, in accordance with an embodiment of the present disclosure.

Like reference numerals refer to corresponding parts throughout the several views of the drawings.

### DETAILED DESCRIPTION OF THE EMBODIMENTS

The embodiments described herein provide systems and methods for predicting polymer sequences using a neural network.

### Introduction

As described above, there is a need in the art for computationally automated protein design methods and workflows that can quickly and efficiently predict properties of residues (e.g., coupling interactions between mutation sites) in a target design region of a polypeptide, and generate accurate polymer sequence designs which satisfy one or more physical objectives.

Software packages for traditional computational protein optimization and design have two essential ingredients: a force-field and/or energy function for ranking the fitness of different protein structures and conformations, and a simulation and/or optimization method to sample and generate these different protein backbone and side-chain conformations. Automated protein sequence design (APSD) includes an additional component in which the composition or sequence identity provides extra degrees of freedom that are co-optimized along with the structure and energy of the protein. Simplifications are made in the treatment of the protein structure to make traditional APSD more feasible.

One popular approach fixes the protein backbone to be immobile, thereby reducing the combinatorial complexity, and in turn the size of the geometric and composition search space. See, for example, Wernisch et al., “Automated protein design with all atom force-fields by exact and heuristic optimization.” J. Mol. Biol. 301, 713-736 (2000); Xiang and Honig, “Extending the Accuracy Limits of Prediction for Side-chain Conformations.” J. Mol. Biol. 311, 421-430 (2001); and Simonson et al., “Computational Protein Design: The Proteus Software and Selected Applications.” J. Comp. Chem. 34, 2472-2484 (2013). Such “fixed-backbone protein design” is typically applied to the re-design of native proteins with well-resolved structures (or a functional scaffold pre-selected based on a backbone structure) with the objective of optimizing a biological function, for example stronger binding affinity to a specified target. With the backbone fixed, there is still the complexity of the side-chain conformational space, which is treated by discretizing the space into a number of well-defined possible conformations, called “rotamers.” See, Xiang and Honig, above. A rotamer is obtained by screening the crystal Protein Data Bank (PDB) database and represents the frequently observed dihedral angle conformations for each individual amino-acid residue type. As a result, a rotamer library will contain, for each residue type, a collection of frequently appearing rotamers. Despite these simplifications to the treatment of a protein structure, crafting an energy function and tuning the parameters within its contributing terms remains a challenge with traditional APSD.

The energy function should be computationally efficient within a combinatorial optimization, especially in the context of variable sequence identity, but also accurate about its predictions of the protein sequence-structure relationship. A poor choice of energy function can lead to poor packing of side-chains in cavities, and an over-abundance of polar amino-acids that tend to replace hydrophobic amino acids, the result of an incorrect relative calibration of protein-protein, protein-solvent interactions as well as entropic effects. The optimization method searches for sequences, while being guided by an energy function which should 1) favor interactions between unlike charges, 2) penalize the burial of polar groups, 3) favor rotamer arrangements that are frequently observed in natural occurring proteins, and 4) reproduce the natural frequencies and/or abundances of different amino-acid types. To achieve the last requirement, energy functions can be augmented with empirical corrections that allow the sequence optimization method to better guide the instantaneous abundances towards natural, PDB abundances (see, Simonson et al., above).

APSD methods are also validated using wild-type sequence and wild-type side-chain recovery tests, achieving reasonable results of about 30% similarity to wild-type sequence. However, since it is difficult to understand how empirical corrections can achieve correct sequence-structure mapping across all protein systems, it is unclear how to systematically improve the sequence recovery. Another shortcoming with traditional APSD methods is their reliance on rotamer databases that, by the very definition of a rotamer, largely omit dynamic, floppy side-chain conformations which yield low electronic densities. Therefore, “rotamers” with important functional conformations may not be correctly sampled. This omission could adversely affect the sequence optimization's capacity to satisfy two or more objectives, for example, maintaining stability while improving binding specificity. Finally, another complication with traditional APSD is how to properly include pH-dependent effects, such as the accurate modeling of protonation state changes and their influence on generated sequences (see, Simonson et al., above).

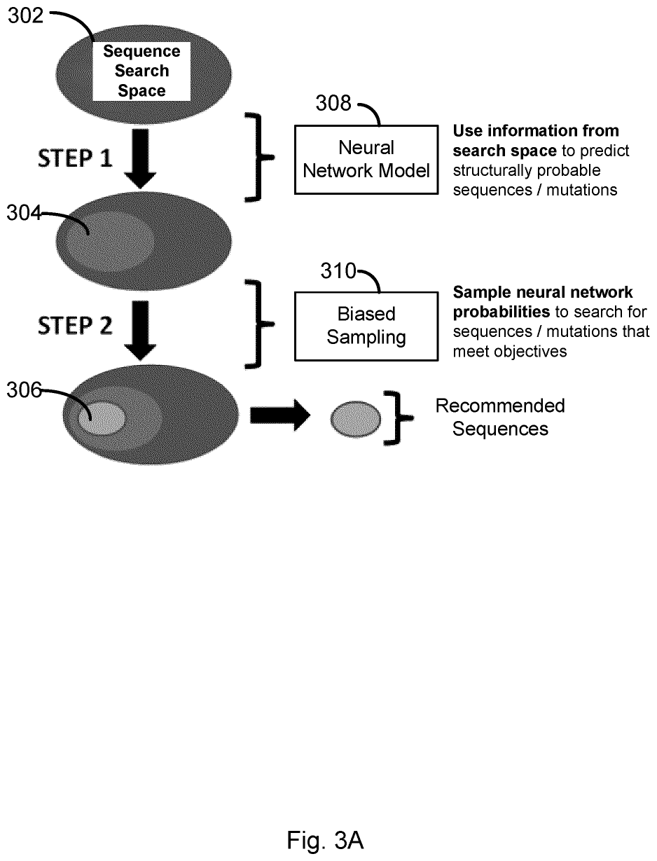

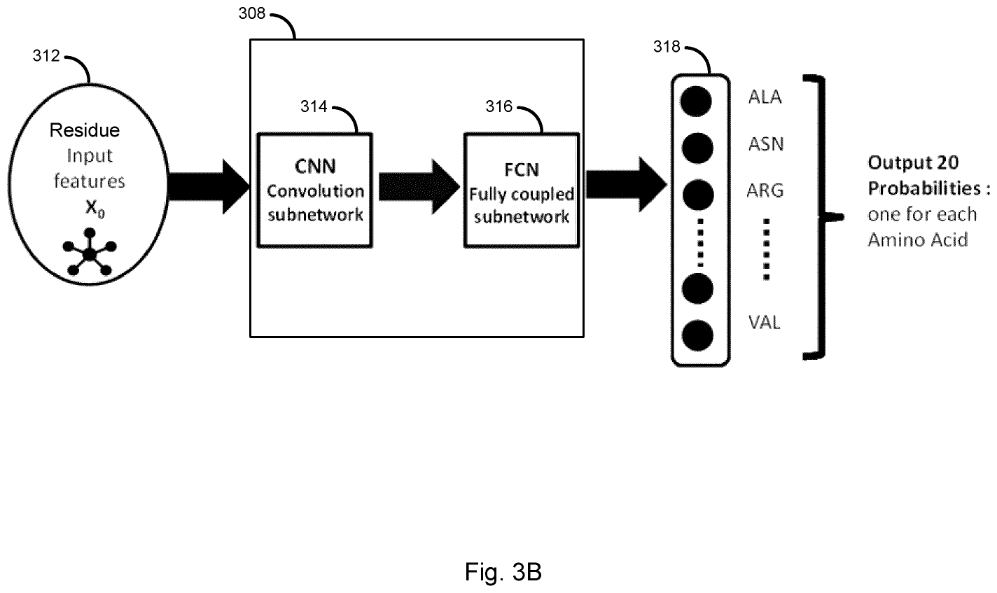

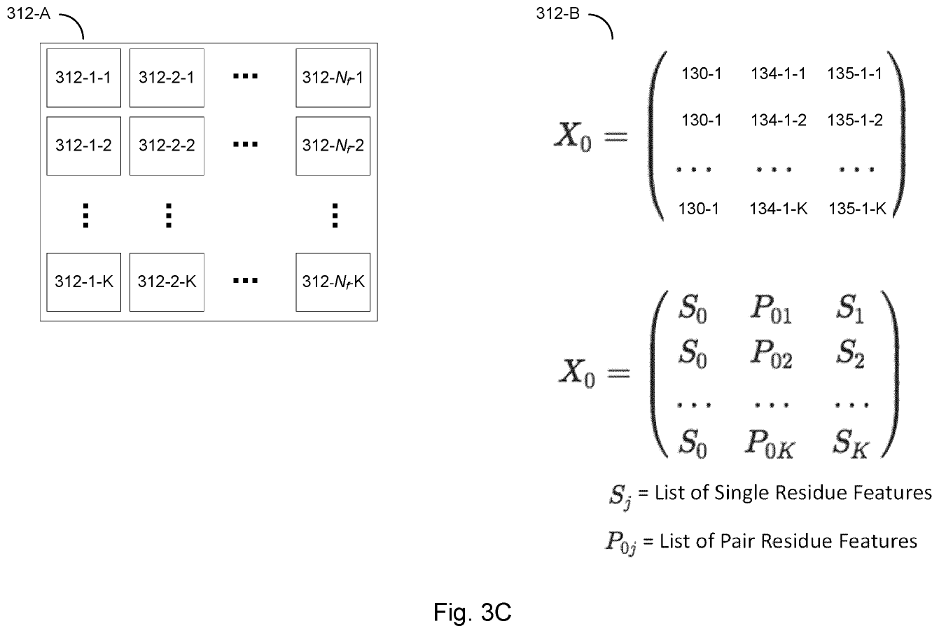

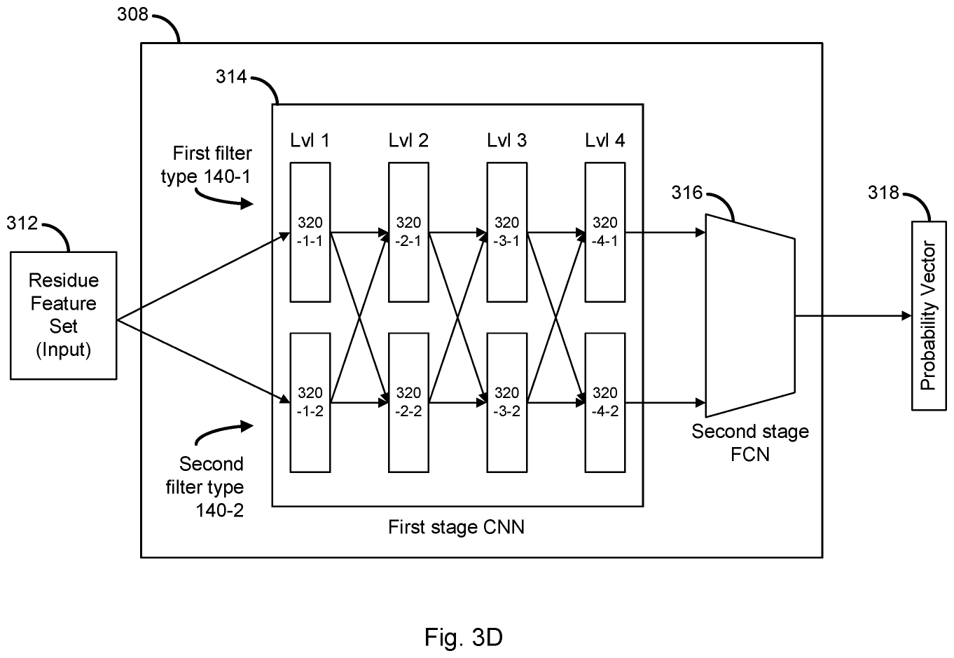

Recently, efforts have been devoted to leveraging deep learning technologies to solve the protein folding problem. See, for example, O'Connell et al., “SPIN2: Predicting sequence profiles from protein structures using deep neural networks.” Proteins 86, 629-633 (2018); Wang et al., “Computational Protein Design with Deep Learning Neural Networks.” Nature. Sci. Rep. 8, 6349 (2018); Cao et al., “DeepDDG: Predicting the Stability Change of Protein Point Mutations Using Neural Networks.” J. Chem. Inf Model 59, 4, 1508-1514 (2019); Qi and Zhang, “DenseCPD: Improving the Accuracy of Neural-Network-Based Computational Protein Sequence Design with DenseNet.” J. Chem. Inf Model 60, 3, 1245-1252 (2020); Senior et al., “Protein structure prediction using multiple deep neural networks in the 13^{th} Critical Assessment of Protein Structure Prediction (CASP 13).” Proteins 87, 1141-1148 (2019); and Xu, “Distance-based protein folding powered by deep learning.” Proc. Natl. Acad. Sci. USA 116, 16856-16865 (2019). For instance, as illustrated by the schematic in FIG. 3A, deep neural network models can be trained on large sequence search spaces 302, such as the vast amounts of sequence and structure data found in the Protein Data Bank (PDB), allowing researchers to discover insights into the intricate dependence of protein sequence on protein structure. As a result, a trained neural network model can be used to significantly reduce the size of the sequence search space from over all possible mutations to over only the most probable, potentially stabilizing mutations 304. Protein engineers can utilize deep neural networks to provide a smaller, more viable sequence/mutation space at the start of their rational protein design process, reducing reliance on a limited set of chemical rules.

In particular, various neural network architectures have been tuned and trained on wild-type protein structures to predict protein sequence. Early architectures included simple, fully-connected neural network models that took, as input, features depending on just backbone geometry; these models relied heavily on choosing a set of pre-engineered input features. More recently, three-dimensional convolution neural networks (3D-CNN) and enormous graph neural network architectures have been developed that more effectively and efficiently learn higher order hierarchical relationships between input features, which consist of atom coordinates, thereby reducing the need for feature pre-engineering. See, e.g., Anand-Achim et al., “Protein sequence design with a learned potential.” biorxiv.org/content/10.1101/2020.01.06.895466v3 (2021); and Ingraham et al., “Generative models for graph-based protein design.” Advances in Neural Information Processing Systems 32, Curran Associates Inc., 15820-15831 (2019). These models have been evaluated and compared mostly based on their ability to recover the protein sequences given only the backbone structure as input information. Typically, these models achieve wild-type sequence recovery rates of about 45 to 55 percent. However, for naturally occurring protein backbones, there is a distribution of sequences that fold into any given target structure, such that it is difficult to determine whether a higher wild-type sequence recovery rate is especially meaningful above a certain threshold. Therefore, alternative approaches to protein optimization and design focus on the generation of sequence distributions (e.g., sequence diversity).

For instance, rather than training on information derived from just backbone geometry or atomic coordinates, one state-of-the-art approach includes training a 3D-CNN model given all backbone and side-chain atoms of those residues neighboring a target residue site within a fixed field of view. The side-chain atoms of the target site were masked out because these atoms pertained to the site of the prediction. The model was then incorporated into a sampling algorithm which cycles through the target residues, updating the amino acid identities at each target residue visited given knowledge of the amino acid identities and side-chain conformations of the surrounding residues. After the assignment of a new amino acid identity at a target residue site, the side-chain conformation at a target residue site must be built subject to the condition that clashes are not introduced with surrounding residues. As a result, this method generally relies on a rotamer repack, using PyRosetta4 (Rosetta Software Suite), applied to the target residue site and surrounding residue sites within a radius of 5 Å from the target. In one implementation, the rotamer repack is not performed but is replaced with a neural network prediction of side-chain conformations; sequence designs are then ranked via a heuristic energy function defined from the probabilities output by their neural networks. See, Anand-Achim et al., above.

Concurrent with the above approach, a second state-of-the-art approach utilizes a graph transformer model that treats the sequence design as a machine translation from an input structure (represented as a graph) to a sequence. The model predicts amino acid identities sequentially using an autoregressive decoder given the structure and previously decoded amino acid identities along a protein chain, proceeding one sequence position to the next in one decoding step, starting from one end of a protein chain and finishing at the other. See, Ingraham et al., above.

As depicted in FIGS. 3A-D, the systems and methods of the present disclosure improve upon the state of the art by providing a workflow for predicting amino acid identities at selected target residue or mutation sites within a protein. In some embodiments, the workflow encompasses two phases used to reduce a traditionally large sequence search space 302 to a subset 306 of recommended polymer sequences and mutations for downstream protein design and optimization. A first phase (“Step 1”) includes using a trained neural network model 308 trained on features (e.g., residue features and/or protein structural features) and labels (e.g., amino acid identities) obtained from the search space 302 (e.g., a protein database). The neural network model 308 predicts sequences and/or mutations (e.g., amino acid identities) that have the highest structural probability, given a set of features 312 (e.g., residue features) for a respective one or more residues 128 of a polypeptide 122, thus reducing the size of the sequence search space 302 from that of all possible mutations for the respective residues to a subset 304 of the most probable, potentially stabilizing mutations. A second phase (“Step 2”) includes using biased sampling 310 (e.g., biased Gibbs deep neural network sampling) over the subset 304 of probable mutations (e.g., amino acid identities derived from the neural network 308). Biased sampling 310 samples neural network probabilities to identify sequences and/or mutations (e.g., amino acid identities) for the respective residues that best meet design goals and objectives, including such properties as stability, affinity, specificity, and/or selectivity. The resulting subset 306 includes the recommended sequences and/or mutations that satisfy objectives for downstream protein design and optimization.

Referring to FIGS. 3B-D, in an example embodiment, the model includes two stages in which, near to its input ports, a first stage 314 comprises a one-dimensional convolution neural (1D-CNN) sub-network architecture. Input 312 for the model includes a residue feature set (e.g., a residue feature data frame) as a K×N_{f} matrix, with a first dimension (e.g., “height”) of K and a second dimension (e.g., “width”) of N_{f}. This first stage 314 CNN sub-network consists of two parallel (e.g., “left” branch and “right” branch), sequential series of convolution layers 320. For instance, in an example implementation illustrated in FIG. 3D, there are four levels (e.g., 320-1-n, 320-2-n, 320-3-n, 320-4-n) in this sub-network and each level consists of two parallel convolution layers (e.g., 320-m−1 and 320-m−2).

At the end of a given level after the activation stage of a convolution for that level, the outputs from two parallel, coincident convolution layers (e.g., the left and right branch layers) are concatenated laterally. In the first stage, each convolution layer is characterized by the number of filters 140 in that layer, as well as the common shape (e.g., height and width) of each of these filters. There are two filter types (e.g., 140-1 and 140-2), each characterized by a distinct “height.” For instance, in the example embodiment, for the “left” branch convolution layer filters, the height is size one; for the “right” branch convolution layer filters, the height is size two. For both filter types, the stride is size one, and the filters when striding run down the height axis of the residue feature data frame. A concatenation yields a new data frame, which in turn serves as the input for the next two parallel, coincident convolution layers (e.g., again including a “left” and a “right” branch) deeper in the overall network. The final concatenation merges the two branches and yields information about the learned higher-order hierarchy of features. In some embodiments, the model comprises, for each respective convolutional layer in the model (e.g., in the first-stage CNN and/or in the second-stage FCN), a respective batch normalization layer that is applied prior to the respective activation stage for the respective convolutional layer.

A 1D-Global Average pooling operation layer is applied to the final concatenated data frame, collapsing this data frame along its height axis; then, the pooled output data frame is inputted into the second stage 316 of the model. In some embodiments, the second stage is a fully-connected traditional neural network. In the example embodiment, the final output of the model consists of a single node that outputs a probability vector 318 including a respective probability 144 for each of a plurality of amino acid identities possible at a target residue site. Each element of the vector is associated with a particular amino acid identity (e.g., Arginine or Alanine), and the vector represents a probability distribution in which the sum of the 20 elements in the vector is equal to one. In some embodiments, an amino acid prediction for the target residue site is made by selecting the amino acid identity for the highest-valued probability element. Parameters 138 in the model are “learned” by training on PDB structure-sequence data.

In some embodiments, the model (e.g., a neural network) takes, as input 312, backbone features or, alternatively, backbone and sidechain features. These features collectively describe the local protein environment “neighboring” a given target residue site and include, but are not limited to, features specific to a respective target residue 130, features specific to a neighboring residue of a respective target residue 134, and/or features specific to a pair of the respective target residue and a neighboring residue 135. In some embodiments, this neighboring environment is defined, for instance, by the K (e.g., a positive integer) nearest neighboring residues to the target residue site based on the C_{α} to C_{α} residue to residue distance. The backbone features, features computed using only the protein backbone atoms, can include the secondary structure, crude relative solvent-accessible surface area (SASA), and backbone torsion angles of collectively all the K neighboring residue sites and the target residue site. In addition, inputs can include geometric and/or edge features like the C_{α} to C_{α} distances between the neighbor sites and the target site, as well as backbone features describing the orientations and positions of the different backbone residue segments of the neighbors relative to the backbone residue segment of the target in a standardized reference frame. This standard frame can be defined so that it is centered on the target residue site: the target's C_{α} atom is chosen as the origin in this local reference frame. The side-chain features can include the amino acid identities of the K-nearest neighboring residues which are encoded (e.g., made numeric) based on their physicochemical properties. This encoding is generated by a “symmetric neural network” used as an encoder-decoder. Once trained, in some embodiments, the encoder learns to represent a higher order (e.g., 7-D physicochemical properties) vector as a lower order (e.g., 2-D) vector. For instance, in an example embodiment, the training dataset for the encoder-decoder consists of a set of twenty 7-D properties vectors, where each of the twenty amino-acid types has a distinct 7-D properties vector.

In some embodiments, the set of input features 312 is obtained using atomic coordinates 124 for the respective polypeptide 122. In some implementations, the set of input features 312 constitutes a simple star graph, which, prior to being fed into the model, are re-shaped into a K×N_{f} matrix where N_{f} is the number of features pertaining to the target residue site and one of its K neighbors. Thus, in some embodiments, a structure of a polypeptide 122 can be represented as a data array of such matrices.

Accordingly, in some embodiments, the model 308 (e.g., the neural network) is trained following a typical machine learning procedure by varying the model's internal parameters 138 so as to minimize a cross-entropy loss function across a training dataset comprising a plurality of protein residue sites (e.g., ˜1,800,000 protein residue sites) and a validation dataset comprising a plurality of protein residue sites (e.g., ˜300,000 protein residue sites) labeled by their amino acid designations. This loss function measures the total cost of errors made by the model in making the amino acid label predictions across a PDB-curated dataset.

Once trained, the model 308 (e.g., the neural network) can be used in a variety of ways. A first application includes predicting amino acid identities at specified target residue sites. A second application includes scoring and/or ranking specified mutations on their ability to improve one or more protein properties of interest, such as stability or protein-protein binding affinity. A third application includes automatically generating novel mutations that could simultaneously improve one or more protein properties, such as stability (e.g., protein fold), binding affinity (e.g., protein-protein binding), binding specificity (e.g., selective protein-protein binding), or a combination thereof. For instance, in some embodiments, the model provides a functional representation for an approximation to the conditional probability of finding an amino acid identity at a single target residue site given a set of input features, with which it is possible to formulate an improved, streamlined neural network-based energy function and in turn generate improved stability and affinity scoring metrics for polymer design.

For example, in some such embodiments, a mutation can be represented as a set of swaps, where each swap consists of an initial and a final amino acid state. The energy and/or stability change due to a mutation (“energy function”) can then be calculated as the sum of contributions from each of its swaps. To evaluate this change, each target residue site 128 in a plurality of target residue sites for the set of swaps in the mutation is sequentially selected and used to obtain probabilities 144 for amino acid swaps using the trained model 308, in accordance with the systems and methods of the present disclosure. The energy/stability contribution from a swap at a target residue site can be defined as the minus of the natural logarithm (−ln( )) of the probability of the final state over the probability of the initial state, where these probabilities 144 are taken from the output 318 of the trained model. For each respective target residue site 128, once a swap has been made, the residue features data frames (e.g., in residue feature data store 126) associated with the other target residue sites in the mutation are updated to reflect the change in the amino acid identity at the respective target residue site. The order in which each target residue site in the plurality of target residue sites is sequentially selected for the swapping and updating is random. Thus, to remove this arbitrariness and make the energy function generally more accurate, the sequential selection of each respective target residue site in the plurality of target residue sites is repeated for a number of iterations (e.g., at least 100, at least 200, or at least 1000 iterations), and the final generated mutations obtained from each iteration in the plurality of iterations can be, e.g., ranked, scored, and/or averaged.

In some embodiments, the model does not provide an output for the joint probability of the amino acid identities across several target residue sites (e.g., the joint probability of multiple residues in a polymer sequence) but rather provides probabilities for amino acid identities or swaps at on a single-site basis. Accordingly, in some embodiments, the present disclosure provides systems and methods for effectively sampling an approximation to the joint probability by cyclically sampling the conditional probability distributions pertaining to each target residue site in the specified target region, using an adaptation of a standard stochastic algorithm such as Gibbs sampling.

For instance, in an example embodiment, a sampling algorithm 310 is performed by randomly assigning an amino acid identity to each target site. The algorithm cycles through the target sites, reassigning amino acid identities based on draws from the conditional probabilities. Each reassignment is referred to as a swap. After a swap at a current target residue site, the input features of those residues having this target as one of its K-nearest neighbors are updated to reflect the new amino acid identity at the target site. This is a stochastic algorithm, in which new amino acid identities are assigned according to draws based on the conditional probability distribution outputted by the model and not on the identity of the maximum-valued probability element. As such, there can be fluctuations in the target region amino acid sequence. The method further comprises repeating the sampling algorithm until the updating of input features (e.g., of target and neighboring residues) based on amino acid swaps reaches convergence. For instance, in some embodiments, the distribution of sequences shifts towards regions of sequence space that are characterized by increased stability with each iteration of the sampling algorithm, such that, upon convergence, the sequences generated by the sampling algorithm are deemed to be stabilizing sequences.

In some embodiments, to meet additional objectives such as enhanced protein binding affinity or specificity, the sampling algorithm 310 is expanded to include a bias (e.g., a Metropolis criterion) involving the additional protein property to be enhanced. This bias (e.g., the Metropolis criterion) imposes a constraint on the sampling in which a drawn swap is not accepted outright, but rather each drawn swap is treated as an “attempted” swap. If the attempted swap leads to enhancement of the additional protein property of interest, then this swap is accepted outright; however, if the attempted swap does not lead to the enhancement of the respective property, then the attempted swap is accepted conditionally, based on a Boltzmann factor which is a function of the potential change to the protein property. In some embodiments, the factor also contains an artificial temperature variable that can be adjusted to control the conditional acceptance of the attempted swap. For instance, in some implementations, attempted swaps that will lead to large declines in the protein property are less likely to be accepted than attempted swaps that will lead to small declines. However, the acceptance of larger declines can be made more likely by increasing the temperature. Thus, the sampling algorithm can be used to generate novel target region sequences (e.g., protein designs) that improve or maintain protein stability while simultaneously enhancing one or more additional protein properties. In some such embodiments, the Boltzmann factor within the Metropolis criterion controls the “drive” of the distribution or ensemble of designs towards an improved value of the one or more additional properties. Once these designs are generated, they can be ranked by stability and affinity metrics either 1) derived from a standard physics-based or knowledge-based forcefield or 2) derived from the neural network energy function based on the conditional probability distributions output by the systems and methods disclosed herein.

In some embodiments, enhanced binding affinity or specificity design simulations utilize the tracking of several chemical species, such as ligands and receptors in bound and unbound states. As a result, in a respective sampling algorithm, when a swap is accepted, for each respective chemical species in a plurality of chemical species affected by the swap, a data array representation (e.g., one or more residue feature sets) for the respective chemical species is updated accordingly. As an illustrative example, enhancement of HetFc binding specificity comprises tracking 7 chemical species including three bound and four unbound chemical species, such that a swap being applied to the heterodimer will also require this swap to correspondingly occur at two residue sites on one of the two homodimers and at one residue site on each of two unbound Fc chains.

### Benefit.

As described above, conventional protein design and optimization is associated with a number of technical problems. For instance, conventional functional protein design is a combinatorial problem with a large search space, where traditional approaches are often computationally expensive or require multiple rounds of design and experimental screening. Adding to this complexity is the problem of multi-objective optimization, where traditional approaches are frequently limited to pairs and triplets of amino acid mutations and have limited ability to predict higher order coupled interactions.

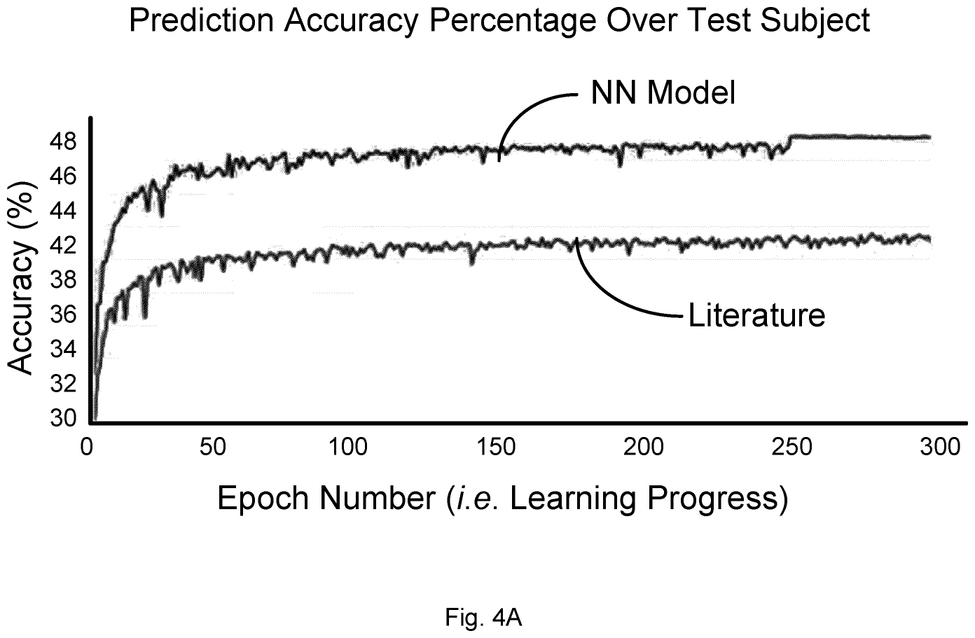

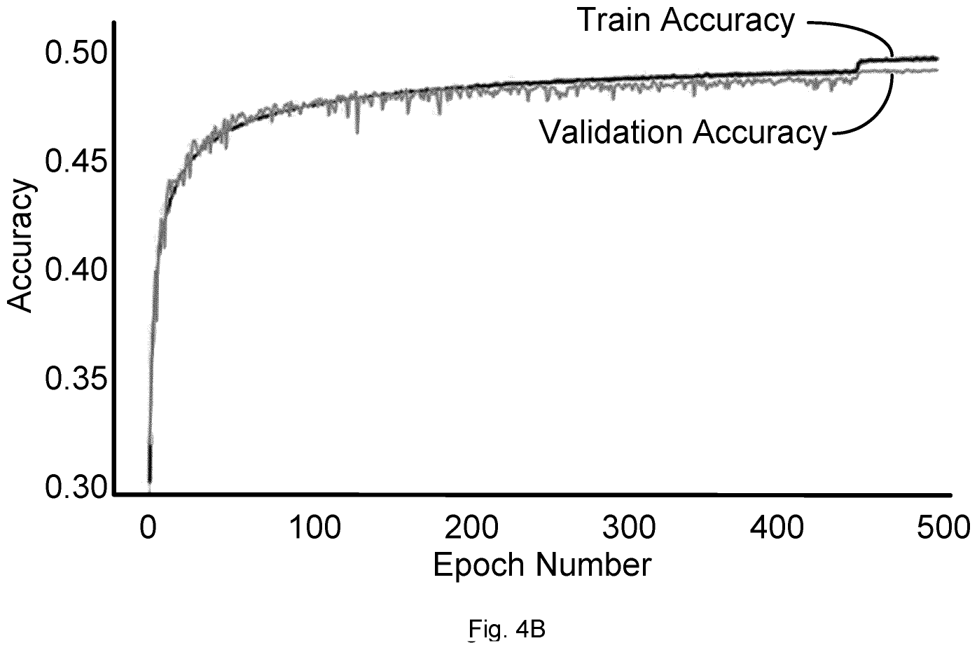

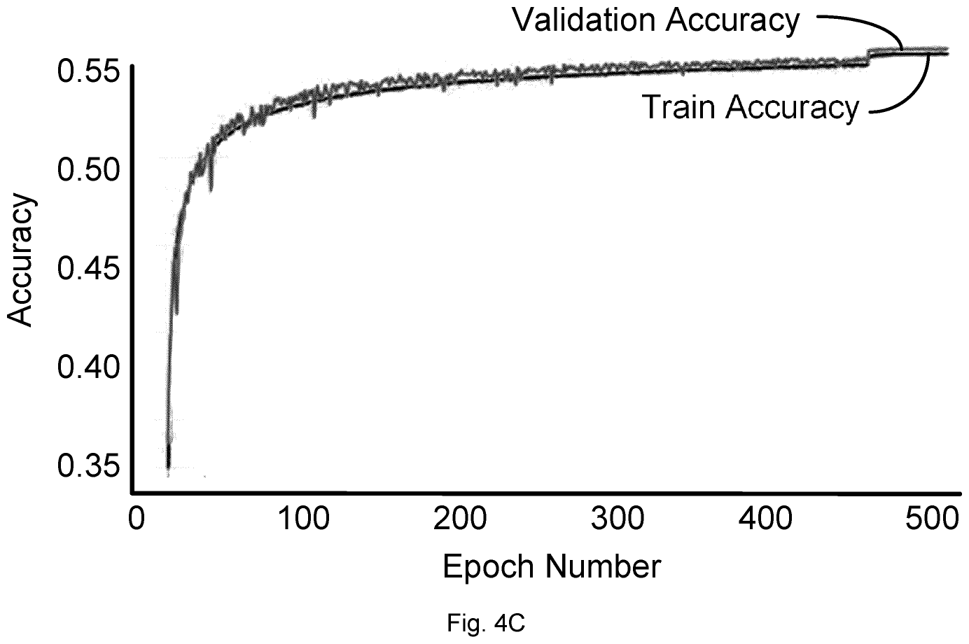

The present disclosure provides solutions to the abovementioned problems by describing systems and methods for predicting an amino acid identity at a target residue site on a polypeptide using a neural network model. Advantageously, the presently disclosed neural network models exhibit comparable accuracy and performance compared to other state-of-the-art neural network models, while reducing the complexity and computational burden typical in such state-of-the-art models due to the simplicity of its architecture. For instance, compared with graph networks trained on atomic coordinates as input features, in some implementations, the neural network models disclosed herein are trained on a small set of local pre-engineered input features and therefore do not require a complex architecture to achieve the comparable level of prediction accuracy. Moreover, compared to traditional computational protein design methods that utilize physical force-fields acting on all-atom protein models, the accuracy of the neural network models of the present disclosure is about 10-15% higher (e.g., approximately 46-49% accuracy) when inputted with only backbone-dependent residue features in wild-type sequence recovery tests. When neighboring residue amino acid identities about a target residue site are added as features, the accuracy of the neural network models of the present disclosure increases to 54-56%. These results are further detailed in Example 1, below, with reference to FIGS. 4A-C.

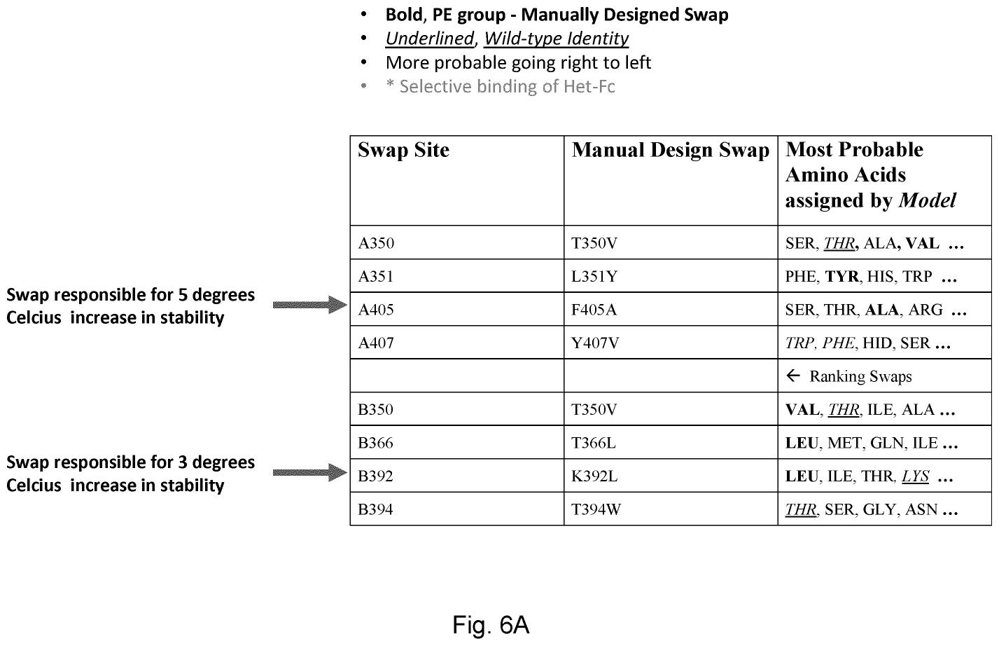

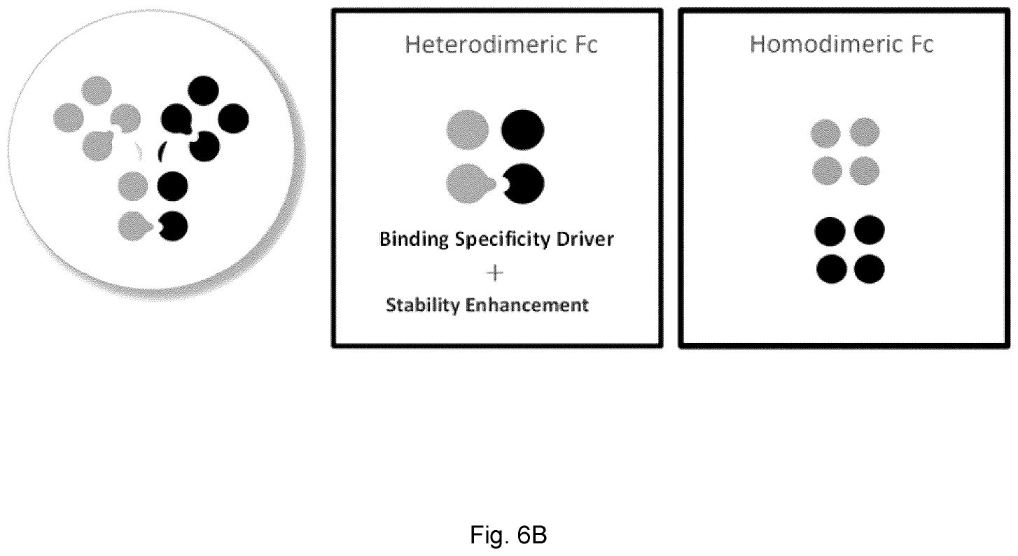

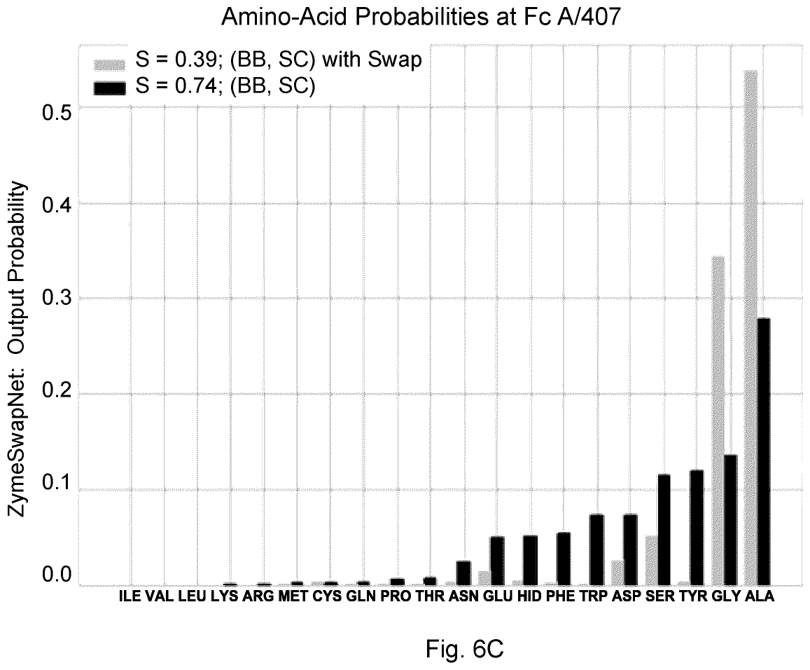

The presently disclosed systems and methods also allow for the effective prediction of polymer sequences, given a polypeptide structure and/or fold (e.g., by repeatedly applying every residue site along the protein backbone to a neural network model). Thus, the presently disclosed systems and methods can be used to discover potentially stabilizing mutations. For instance, as illustrated in Example 2 with reference to FIGS. 6A-C, a neural network model in accordance with the present disclosure was able to correctly predict amino acid swaps that replicated or were similar to corrective stabilizing swaps previously identified by protein engineers during heterodimeric Fc design.

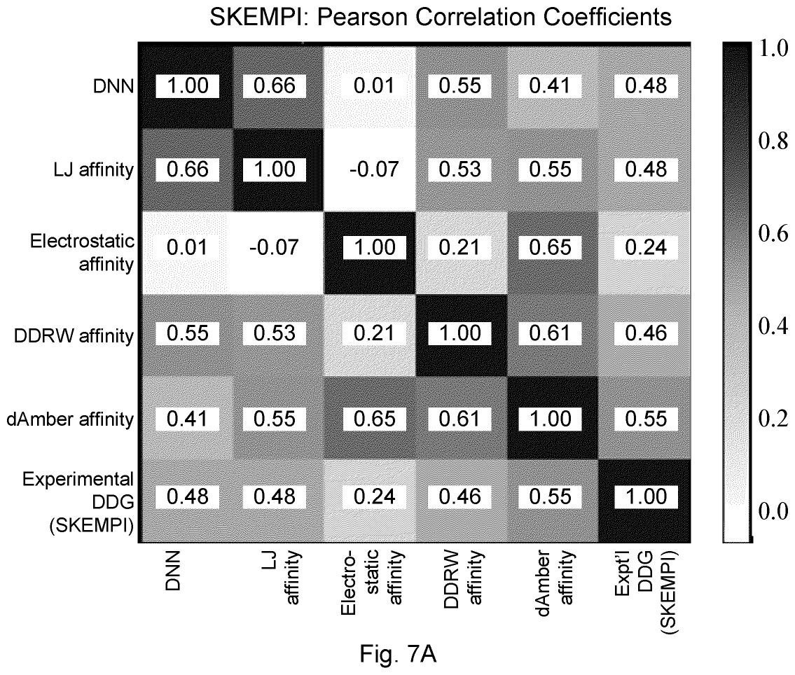

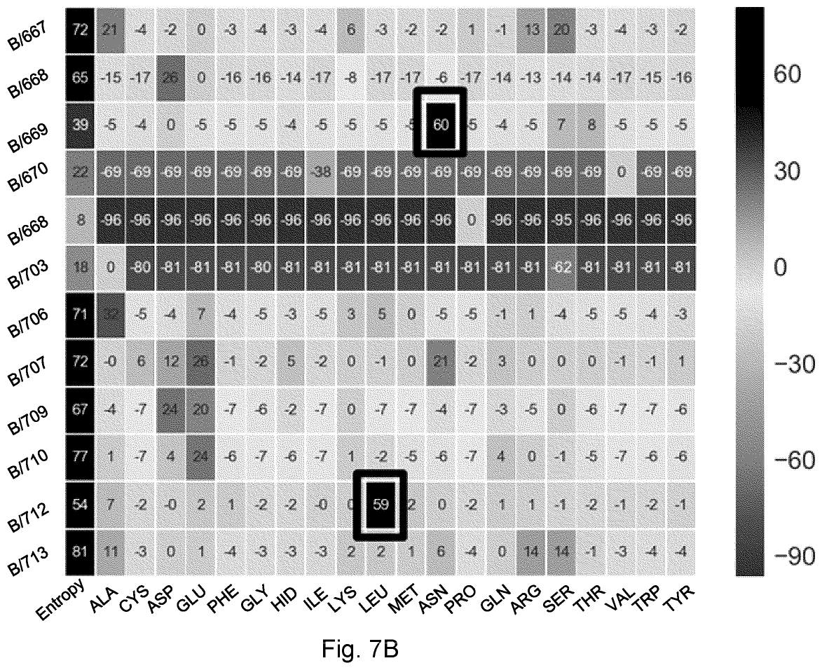

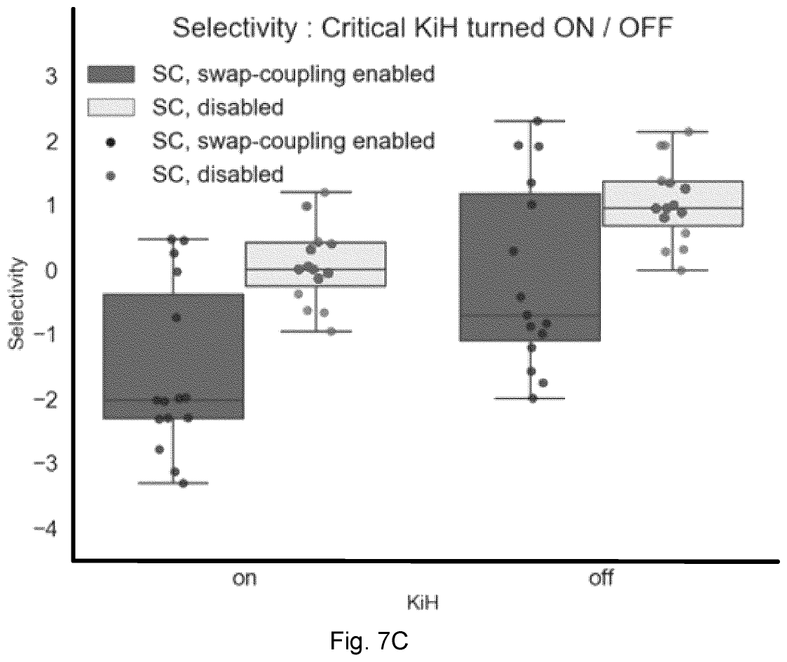

The probability distributions output by the neural network model can be used to formulate neural network-based stability and affinity metrics to score and rank mutations, which correlated well (Pearson r˜0.5, Kendall Tau ˜0.3) with experimental protein binding affinity measurements obtained from a conventional, open-source protein-protein affinity database (SKEMPI). See, for example, FIG. 7A and Jankauskaité et al., “SKEMPI 2.0: an updated benchmark of changes in protein-protein binding energy, kinetics and thermodynamics upon mutation.” Bioinformatics 35(3), 462-469 (2019), which is hereby incorporated herein by reference in its entirety. Unlike the calculation of other comparable standard physics-based (e.g., Amber force-field) and knowledge-based, statistical affinity metrics, the metrics obtained using the presently disclosed neural network models are extremely fast because they do not require a lengthy side-chain conformational repacking. Furthermore, as a result, the neural network-derived metric calculations avoid being beleaguered by issues associated with repacking, such as the choice of force-field, the treatment of protonation state, and/or the potential deficiencies arising from the reliance on a rotamer library. This makes the calculation of neural network stability and affinity metrics, as disclosed herein, beneficial for fast inspections on the impacts of set of mutations applied to a starting protein structure. For instance, a trained neural network in accordance with some embodiments of the present disclosure is able to generate polymer sequence predictions for an entire Fc or Fab region of an antibody in less than one minute.

As described above, in some embodiments, the presently disclosed systems and methods can be used to quickly and efficiently rank mutations, as well as in the context of a sampling algorithm within a computational automated sequence design workflow to rapidly propose novel mutations and/or designs. In some embodiments, these workflows can be constructed so that the output mutations and/or designs satisfy one or multiple protein design objectives. Advantageously, in some embodiments, the sampling workflow can be performed using only a starting design structure and a list of target regions to be sampled over as input, in order to generate favorable target region sequences. Moreover, in some embodiments, there is no restriction to the selection of these residues; that is, the residues in the target region do not have to be contiguous in protein sequence position.

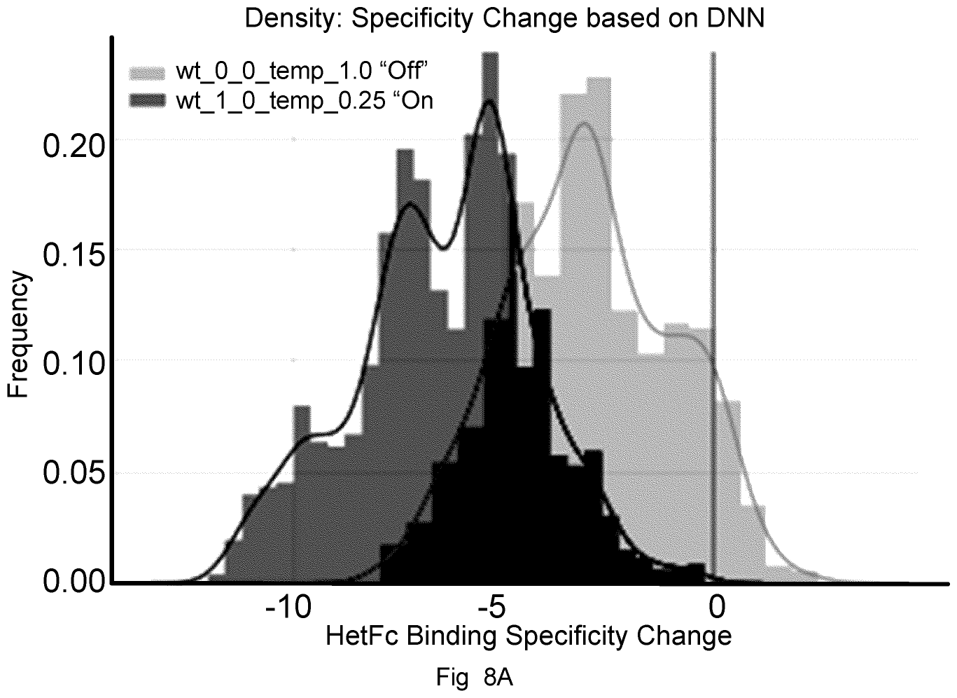

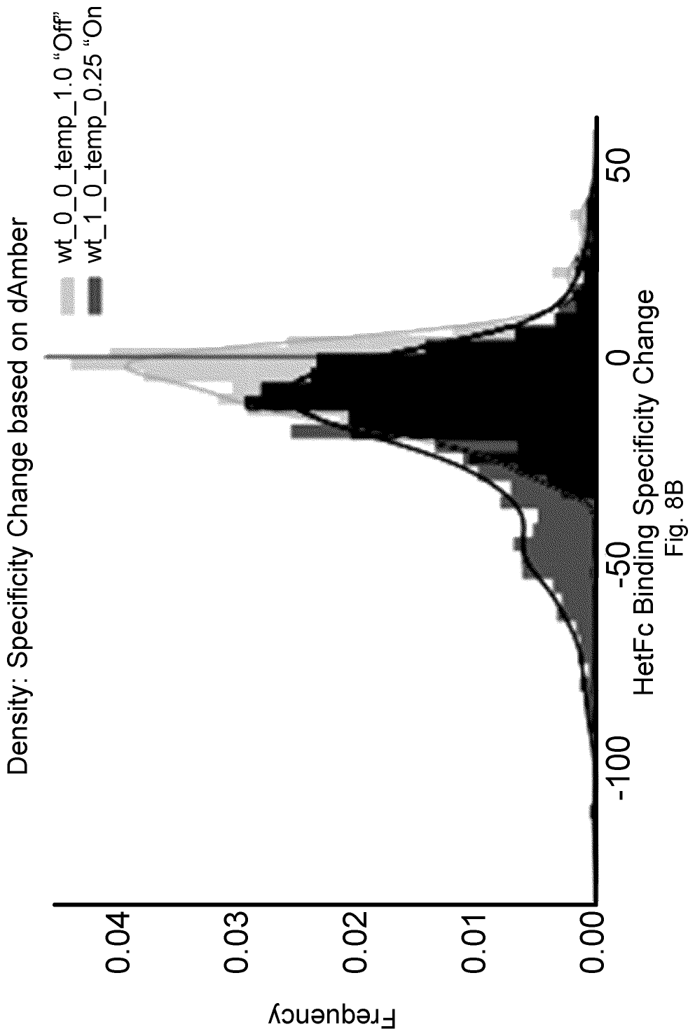

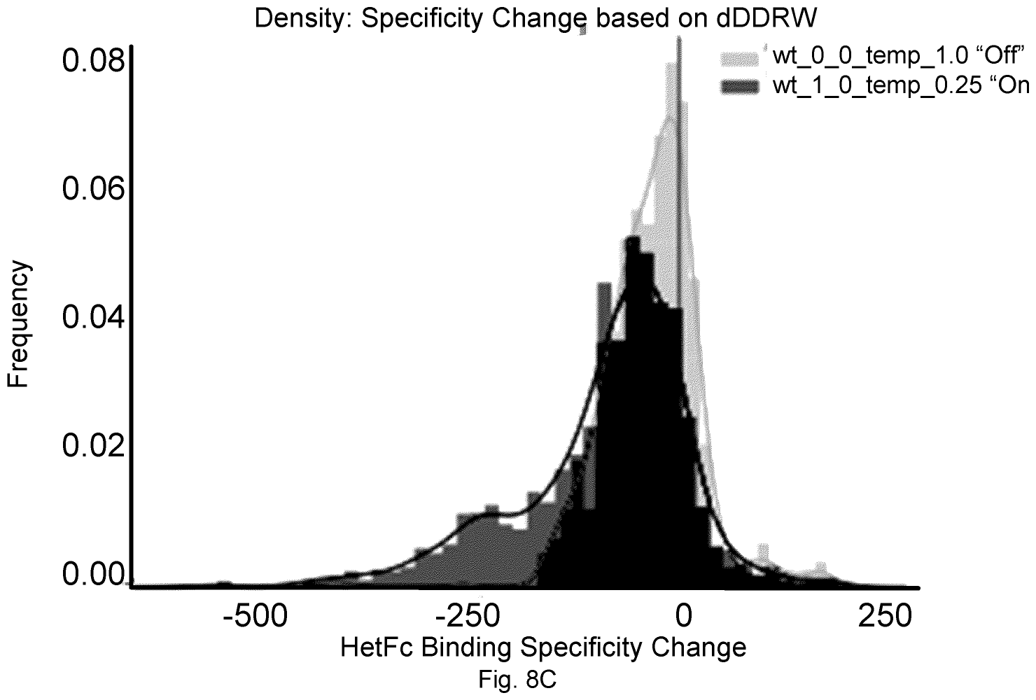

An illustrative demonstration is the design of a HetFc where the two objectives are the enhancements to stability of the HetFc and the binding specificity to this heterodimer over the two competing homodimers (HomoFc). By performing the sampling workflow for a selection of target regions spanning the Fc interface (see, e.g., Von Kreudenstein et al., “Improving biophysical properties of a bispecific antibody scaffold to aid developability.” mAbs 5, 5, 646-654 (2013)), it was determined that, as the Metropolis specificity bias was increased, the distribution of generated HetFc designs shifted towards specificity metric values that heavily favored HetFc over HomoFc binding. The stochastic sampling algorithm, which incorporates the conditional probability outputted by the neural network model to make instantaneous amino acid assignments at target residue sites, was found capable of generating novel HetFc designs as well as designs similar to those discovered by protein engineers using multiple rounds of rational design and experimental verification. As illustrated in Example 3 with reference to FIGS. 8A-C and 9A-C, specificity metric values representing a more favorable binding of HetFc over HomoFc exhibited a positive correlation with HetFc purity of polymer designs, thereby demonstrating another correlation of the presently disclosed neural network models with experimental data.

The present disclosure therefore provides computational tools that, using a reduced set of structural and physicochemical inputs, automate the discovery of beneficial polymer sequences and mutations to reduce the workload and time of performing conventional, manual protein engineering and mutagenesis projects. Moreover, these tools provide more accurate recommendations, scoring, ranking and/or identification of candidate sequences and mutations that are potentially useful, thereby defining a search space of such recommended sequences that, in some embodiments, satisfy multiple design objectives including stability, affinity, and/or specificity.

Regarding the architecture of the neural network models, the present disclosure further provides various systems and methods that improve the computational elucidation of polymer sequences by improving the training and use of a model for more accurate amino acid prediction and sequence generation. The complexity of a machine learning model includes time complexity (running time, or the measure of the speed of an algorithm for a given input size n), space complexity (space requirements, or the amount of computing power or memory needed to execute an algorithm for a given input size n), or both. Complexity (and subsequent computational burden) applies to both training of and prediction by a given model.

In some instances, computational complexity is impacted by implementation, incorporation of additional algorithms or cross-validation methods, and/or one or more parameters (e.g., weights and/or hyperparameters). In some instances, computational complexity is expressed as a function of input size n, where input data is the number of instances (e.g., the number of training examples such as residues, target regions, and/or polypeptides), dimensions p (e.g., the number of residue features), the number of trees nₜᵣₑₑₛ (e.g., for methods based on trees), the number of support vectors nₛᵥ (e.g., for methods based on support vectors), the number of neighbors k (e.g., for k nearest neighbor algorithms), the number of classes c, and/or the number of neurons n, at a layer i (e.g., for neural networks). With respect to input size n, then, an approximation of computational complexity (e.g., in Big O notation) denotes how running time and/or space requirements increase as input size increases. Functions can increase in complexity at slower or faster rates relative to an increase in input size. Various approximations of computational complexity include but are not limited to constant (e.g., O(1)), logarithmic (e.g., O(log n)), linear (e.g., O(n)), loglinear (e.g., O(n log n)), quadratic (e.g., O(n²)), polynomial (e.g., O(n^{c})), exponential (e.g., O(cⁿ)), and/or factorial (e.g., O(n!)). In some instances, simpler functions are accompanied by lower levels of computational complexity as input sizes increase, as in the case of constant functions, whereas more complex functions such as factorial functions can exhibit substantial increases in complexity in response to slight increases in input size.

Computational complexity of machine learning models can similarly be represented by functions (e.g., in Big O notation), and complexity may vary depending on the type of model, the size of one or more inputs or dimensions, usage (e.g., training and/or prediction), and/or whether time or space complexity is being assessed. For example, complexity in decision tree algorithms is approximated as O(n²p) for training and O(p) for predictions, while complexity in linear regression algorithms is approximated as O(p²n+p³) for training and O(p) for predictions. For random forest algorithms, training complexity is approximated as O(n^{Z}pnₜᵣₑₑₛ) and prediction complexity is approximated as O(pnₜᵣₑₑₛ). For gradient boosting algorithms, complexity is approximated as O(npnₜᵣₑₑₛ) for training and O(pnₜᵣₑₑₛ) for predictions. For kernel support vector machines, complexity is approximated as O(n²p+n³) for training and O(ns˜p) for predictions. For naïve Bayes algorithms, complexity is represented as O(np) for training and O(p) for predictions, and for neural networks, complexity is approximated as O(pn₁+n₁n₂+ . . . ) for predictions. Complexity in K nearest neighbors algorithms is approximated as O(knp) for time and O(np) for space. For logistic regression algorithms, complexity is approximated as O(np) for time and O(p) for space. For logistic regression algorithms, complexity is approximated as O(np) for time and O(p) for space.

Accordingly, for machine learning models, computational complexity dictates the scalability and therefore the overall effectiveness and usability of a model (e.g., a neural network) for increasing input, feature, and/or class sizes, as well as for variations in model architecture. As discussed above, in the context of functional protein design, where the space of all possible structural and chemical changes for a respective protein structure is potentially enormous, the computational complexity of functions performed on such a large search space may strain the capabilities of many existing systems. In addition, as the number of input features (e.g., residue features, neighboring residue features, and/or pair residue features) and/or the number of instances (e.g., training examples, residues, target regions, and/or polypeptides) increases with expanding downstream applications and possibilities, the computational complexity of any given classification model can quickly overwhelm the time and space capacities provided by the specifications of a respective system.

Thus, by using a machine learning model with a minimum input size (e.g., at least 10, at least 50, or at least 100 features in a corresponding residue feature set for a respective residue) and/or a minimum number of parameters (e.g., at least 100, at least 500, or at least 1000 parameters) for polymer sequence prediction, the computational complexity is proportionally increased such that it cannot be mentally performed, and the method addresses a computational problem.

Additional details on computational complexity in machine learning models are provided in “Computational complexity of machine learning algorithms,” published Apr. 16, 2018, available online at: thekerneltrip.com/machine/learning/computational-complexity-learning-algorithms; Hastie, 2001, The Elements of Statistical Learning, Springer, New York; and Arora and Barak, 2009, Computational Complexity: A Modern Approach, Cambridge University Press, New York; each of which is hereby incorporated herein by reference in its entirety.

Still other benefits of the sampling and simulation methods disclosed herein include the ability to utilize such schemes with a variety of suitable neural network architectures, including, but not limited to, for example, graph neural networks trained to predict amino acid identities using input residue features.

### REFERENCES

### Protein Folding

A. W. Senior, R. Evans, J. Jumper, J. Kirkpatrick, L. Sifre, T. Green, Q. Chongli, A. Zidek, A. W. R. Nelson, A. Bridgland, et al., Protein structure prediction using multiple deep neural networks in the 13^{th} Critical Assessment of Protein Structure Prediction (CASP 13). Proteins 87, 1141-1148 (2019).
J. Xu. Distance-based protein folding powered by deep learning. Proc. Natl. Acad. Sci. USA 116, 16856-16865 (2019).

### Traditional Automated Protein Sequence Design

L. Wernisch, S. Hery, S. J. Wodak, Automated protein design with all atom force-fields by exact and heuristic optimization. J. Mol. Biol. 301, 713-736 (2000).
Z. Xiang, B. Honig, Extending the Accuracy Limits of Prediction for Side-chain Conformations. J Mol. Biol. 311, 421-430 (2001).
T. Simonson, T. Gaillard, D. Mignon, M. S. am Busch, A. Lopes, N. Amara, S. Polydorides, A. Sedano, K. Druart, G. Archontis, Computational Protein Design: The Proteus Software and Selected Applications. J Comp. Chem. 34, 2472-2484 (2013).

### Multi-Objective Specificity Design

J. J. Havranek, Specificity in Computational Protein Design. J. Biol. Chem. 285, 41, 31095-31099 (2010).
A. Leaver-Fay, R. Jacak, P. B. Stranges, B. Kuhlman, A Generic Program for Multistate Protein Design. PLoS ONE 6, 7, e20937 (2011).
A. Leaver-Fay, K. J. Froning, S. Atwell, H. Aldaz, A. Pustilnik, F. Lu, F. Huang, R. Yuan, S. Hassanali, A. K. Chamberlain, J. R. Fitchett, S. J. Demarest, B. Kuhlman, Computationally Designed Bispecific Antibodies Using Negative State Repertoires. Structure 24, 4, 641-651 (2016).

### Deep Learning Models

J. O'Connell, Z. Li, J. Hanson, R. Heffernan, J. Lyons, K. Paliwal, A. Dehzangi, Y. Yang, Y. Zhou. SPIN2: Predicting sequence profiles from protein structures using deep neural networks. Proteins 86, 629-633 (2018).
J. Wang, H. Cao, J. Z. H. Zhang, Y. Qi, Computational Protein Design with Deep Learning Neural Networks. Nature. Sci. Rep. 8, 6349 (2018).
H. Cao, J. Wang, L. He, Y. Qi, J. Z. Zhang, DeepDDG: Predicting the Stability Change of Protein Point Mutations Using Neural Networks. J. Chem. Inf Model 59, 4, 1508-1514 (2019).
Y. Qi, J. Z. H. Zhang, DenseCPD: Improving the Accuracy of Neural-Network-Based Computational Protein Sequence Design with DenseNet. J. Chem. Inf Model 60, 3, 1245-1252 (2020).
J. Meiler, M. Müller, A. Zeidler, F. Schmaschke, Generation and evaluation of dimension-reduced amino-acid parameter representations by artificial neural networks. J. Mol. Model 7, 360-369 (2001).

### Sampling Methods

N. Anand-Achim, R. R. Eguchi, I. I. Mathews, C. P. Perez, A. Derry, R. B. Altman, P.-S. Huang, Protein sequence design with a learned potential. Available on the Internet at biorxiv.org/content/10.1101/2020.01.06.895466v3 (2021).
J. Ingraham, V. K. Garg, R. Barzilay, T. Jaakkola, Generative models for graph-based protein design. Advances in Neural Information Processing Systems 32, Curran Associates Inc., 15820-15831 (2019).

### HetFc and Bispecific Design

J. B. B. Ridgeway, L. G. Presta, and P. Carter, “Knobs-into-Holes” engineering of antibody CH3 domains for heavy chain heterodimerization. Protein Engineering vol. 9 no. 7 pp. 617-621 (1996).
P. Carter, Bispecific human IgG by design. J. Immuno. Methods 248, 7-15 (2001).
K. Gunasekaran, M. Pentony, M. Shen, L. Garrett, C. Forte, A. Woodward, S. B. Ng, T. Born, M. Retter, K. Manchulenko, H. Sweet, I. N. Foltz, M. Wittekind, W. Yan, Enhancing Antibody Fc Heterodimer Formation through Electrostatic Steering Effects. J Biol. Chem. 285, 25, 19637-19646 (2010).
T. S. Von Kreudenstein, E. Escobar-Carbrera, P. I. Lario, I D'Angelo, K. Brault, J. F. Kelly, Y. Durocher, J. Baardsnes, R. J. Woods, M. H. Xie, P-A. Girod, M. D. L. Suits, M. J. Boulanger, D. K. Y. Poon, G. Y. Ng, S. B. Dixit, Improving biophysical properties of a bispecific antibody scaffold to aid developability. mAbs 5, 5, 646-654 (2013).
J-H. Ha, J-E. Kim, Y-S. Kim, Immunoglobulin Fc Heterodimer Platform Technology: From Design to Applications in Therapeutic Antibodies and Proteins. Front Immunol. 7, 394 (2016).

### Affinity Dataset

J. Jankauskaité, B. Jimémez-García, J. Dapkunas, J. Fernández-Recio, I. H. Moal, SKEMPI 2.0: an updated benchmark of changes in protein-protein binding energy, kinetics and thermodynamics upon mutation. Bioinformatics 35(3), 462-469 (2019).

### Definitions

As used herein, the term “amino acid” refers to naturally occurring and non-natural amino acids, including amino acid analogs and amino acid mimetics that function in a manner similar to the naturally occurring amino acids. Naturally occurring amino acids include those encoded by the genetic code, as well as those amino acids that are later modified, e.g., hydroxyproline, y-carboxyglutamate, and O-phosphoserine. Naturally occurring amino acids can include, e.g., D- and L-amino acids. The amino acids used herein can also include non-natural amino acids. Amino acid analogs refer to compounds that have the same basic chemical structure as a naturally occurring amino acid, e.g., any carbon that is bound to a hydrogen, a carboxyl group, an amino group, and an R group, e.g., homoserine, norleucine, methionine sulfoxide, or methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “backbone” refers to a contiguous chain of covalently bound atoms (e.g., carbon, oxygen, or nitrogen atom) or moieties (e.g., amino acid or monosaccharide), in which removal of any of the atoms or moiety would result in interruption of the chain.

As used herein the term “classification” can refer to any number(s) or other characters(s) that are associated with a particular property of a polymer and/or an amino acid. For example, a “+” symbol (or the word “positive”) can signify that a respective naturally occurring amino acid is classified as having the greatest probability for an identity of a respective residue. In another example, the term “classification” can refer to a probability for an amino acid in a plurality of probabilities for amino acids. The classification can be binary (e.g., positive or negative, yes or no) or have more levels of classification (e.g., a value from 1 to 10, from 0 to 100, and/or from 0 to 1). The terms “cutoff” and “threshold” can refer to predetermined numbers used in an operation. For example, a threshold value can be a value above or below which a particular classification applies. Either of these terms can be used in either of these contexts.

As used interchangeably herein, the term “model” or “classifier” refers to a machine learning model or algorithm. In some embodiments, a classifier is an unsupervised learning algorithm. One example of an unsupervised learning algorithm is cluster analysis.

In some embodiments, a classifier is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a classifier is a multinomial classifier algorithm. In some embodiments, a classifier is a 2-stage stochastic gradient descent (SGD) model. In some embodiments, a classifier is a deep neural network (e.g., a deep-and-wide sample-level classifier).

Neural networks. In some embodiments, the classifier is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of neurons (interchangeably, “nodes”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xᵢ, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in predicting polymer sequences. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used for predicting polymer sequences in accordance with the present disclosure.

For instance, a deep neural network classifier comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network classifier. In some embodiments, at least 100 parameters, at least 1000 parameters, at least 2000 parameters or at least 5000 parameters are associated with the deep neural network classifier. As such, deep neural network classifiers require a computer to be used because they cannot be mentally solved. In other words, given an input to the classifier, the classifier output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as classifiers are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as classifiers are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as classifiers are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the classifier is a support vector machine (SVM). SVM algorithms suitable for use as classifiers are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM classifier requires a computer to calculate because it cannot be mentally solved.

Naïve Bayes algorithms. In some embodiments, the classifier is a Naive Bayes algorithm. Naïve Bayes classifiers suitable for use as classifiers are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes classifier is any classifier in a family of “probabilistic classifiers” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a classifier is a nearest neighbor algorithm. Nearest neighbor classifiers can be memory-based and include no classifier to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. Here, the distance to these neighbors is a function of the abundance values of the discriminating gene set. In some embodiments, Euclidean distance in feature space is used to determine distance as d₍ᵢ₎=∥x₍ᵢ₎−x₍ₒ₎∥. Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor classifier is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor classifier is such that a computer is used to solve the classifier for a given input because it cannot be mentally performed.

Random forest, decision tree, and boosted tree algorithms. In some embodiments, the classifier is a decision tree. Decision trees suitable for use as classifiers are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree classifier includes at least 10, at least 20, at least 50, or at least 100 parameters (e.g., weights and/or decisions) and requires a computer to calculate because it cannot be mentally solved.

Regression. In some embodiments, the classifier uses a regression algorithm. A regression algorithm can be any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the classifier. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the classifier makes use of a regression model disclosed in Hastie et al., 2001, The Elements of Statistical Learning, Springer-Verlag, New York. In some embodiments, the logistic regression classifier includes at least 10, at least 20, at least 50, at least 100, or at least 1000 parameters (e.g., weights) and requires a computer to calculate because it cannot be mentally solved.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the classifier (linear classifier) in some embodiments of the present disclosure.

Mixture model and Hidden Markov model. In some embodiments, the classifier is a mixture model, such as that described in McLachlan et al., Bioinformatics 18(3):413-422, 2002. In some embodiments, in particular, those embodiments including a temporal component, the classifier is a hidden Markov model such as described by Schliep et al., 2003, Bioinformatics 19(1):i255-i263.

Clustering. In some embodiments, the classifier is an unsupervised clustering model. In some embodiments, the classifier is a supervised clustering model. Clustering algorithms suitable for use as classifiers are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. However, clustering may not use of a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

Ensembles of classifiers and boosting. In some embodiments, an ensemble (two or more) of classifiers is used. In some embodiments, a boosting technique such as AdaBoost is used in conjunction with many other types of learning algorithms to improve the performance of the classifier. In this approach, the output of any of the classifiers disclosed herein, or their equivalents, is combined into a weighted sum that represents the final output of the boosted classifier. In some embodiments, the plurality of outputs from the classifiers is combined using any measure of central tendency known in the art, including but not limited to a mean, median, mode, a weighted mean, weighted median, weighted mode, etc. In some embodiments, the plurality of outputs is combined using a voting method. In some embodiments, a respective classifier in the ensemble of classifiers is weighted or unweighted.

As used herein, the term “parameter” refers to any coefficient or, similarly, any value of an internal or external element (e.g., a weight and/or a hyperparameter) in a model (e.g., an algorithm, regressor, and/or classifier) that can affect (e.g., modify, tailor, and/or adjust) one or more inputs, outputs, and/or functions in the model. For example, in some embodiments, a parameter refers to any coefficient, weight, and/or hyperparameter that can be used to affect the behavior, learning and/or performance of a model. In some instances, a parameter is used to increase or decrease the influence of an input (e.g., a feature) to a model. As a nonlimiting example, in some instances, a parameter is used to increase or decrease the influence of a node (e.g., of a neural network), where the node includes one or more activation functions. Assignment of parameters to specific inputs, outputs, and/or functions is not limited to any one paradigm for a given model but can be used in any suitable model architecture for a desired performance. In some embodiments, a parameter has a fixed value. In some embodiments, a value of a parameter is manually and/or automatically adjustable. In some embodiments, a value of a parameter is modified by a validation and/or training process for a model (e.g., by error minimization and/or backpropagation methods, as described elsewhere herein).

In some embodiments, a model of the present disclosure comprises a plurality of parameters. In some embodiments the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500: n≥10.000: n≥20.000: n≥40,000; n≥75.000: n≥100.000: n≥200.000: n≥500,000, n≥1×10⁶, n≥5×10⁶, or n≥1×10⁷. In some embodiments n is between 10,000 and 1×10⁷, between 100,000 and 5×10⁶, or between 500,000 and 1×10⁶.

As used herein, the term “polymer” refers to a large molecular system composed of repeating structural units. These repeating structural units are termed particles or residues interchangeably herein. In some embodiments, each particle pᵢ in the set of {pᵢ, . . . , p_{K}} particles represents a single different residue in the native polymer. To illustrate, consider the case where a native polymer comprises 100 residues. In this instance, the set of {pᵢ, . . . , p_{K}} comprises 100 particles, with each particle in {pᵢ, . . . , p_{K}} representing a different one of the 100 particles.

In some embodiments, a polymer comprises between 2 and 5,000 residues, between 20 and 50,000 residues, more than 30 residues, more than 50 residues, or more than 100 residues. In some embodiments, a residue in a respective polymer comprises two or more atoms, three or more atoms, four or more atoms, five or more atoms, six or more atoms, seven or more atoms, eight or more atoms, nine or more atoms or ten or more atoms. In some embodiments a polymer has a molecular weight of 100 Daltons or more, 200 Daltons or more, 300 Daltons or more, 500 Daltons or more, 1000 Daltons or more, 5000 Daltons or more, 10,000 Daltons or more, 50,000 Daltons or more or 100,000 Daltons or more.

In some embodiments, a polymer is a natural material. In some embodiments, a polymer is a synthetic material. In some embodiments, a polymer is an elastomer, shellac, amber, natural or synthetic rubber, cellulose, Bakelite, nylon, polystyrene, polyethylene, polypropylene, or polyacrylonitrile, polyethylene glycol, or polysaccharide.

In some embodiments, a polymer is a heteropolymer (copolymer). A copolymer is a polymer derived from two (or more) monomeric species, as opposed to a homopolymer where only one monomer is used. Copolymerization refers to methods used to chemically synthesize a copolymer. Examples of copolymers include, but are not limited to, ABS plastic, SBR, nitrile rubber, styrene-acrylonitrile, styrene-isoprene-styrene (SIS) and ethylene-vinyl acetate. Since a copolymer consists of at least two types of constituent units (also structural units, or particles), copolymers can be classified based on how these units are arranged along the chain. These include alternating copolymers with regular alternating A and B units. See, for example, Jenkins, 1996, “Glossary of Basic Terms in Polymer Science,” Pure Appl. Chem. 68 (12): 2287-2311, which is hereby incorporated herein by reference in its entirety. Additional examples of copolymers are periodic copolymers with A and B units arranged in a repeating sequence (e.g., (A-B-A-B-B-A-A-A-A-B-B-B)ₙ). Additional examples of copolymers are statistical copolymers in which the sequence of monomer residues in the copolymer follows a statistical rule. If the probability of finding a given type monomer residue at a particular point in the chain is equal to the mole fraction of that monomer residue in the chain, then the polymer may be referred to as a truly random copolymer. See, for example, Painter, 1997, Fundamentals of Polymer Science, CRC Press, 1997, p 14, which is hereby incorporated by reference herein in its entirety. Still other examples of copolymers that may be evaluated using the disclosed systems and methods are block copolymers comprising two or more homopolymer subunits linked by covalent bonds. The union of the homopolymer subunits may require an intermediate non-repeating subunit, known as a junction block. Block copolymers with two or three distinct blocks are called diblock copolymers and triblock copolymers, respectively.

In some embodiments, the polymer is a plurality of polymers, where the respective polymers in the plurality of polymers do not all have the molecular weight. In such embodiments, the polymers in the plurality of polymers fall into a weight range with a corresponding distribution of chain lengths. In some embodiments, the polymer is a branched polymer molecular system comprising a main chain with one or more substituent side chains or branches. Types of branched polymers include, but are not limited to, star polymers, comb polymers, brush polymers, dendronized polymers, ladders, and dendrimers. See, for example, Rubinstein et al., 2003, Polymer physics, Oxford; New York: Oxford University Press. p. 6, which is hereby incorporated by reference herein in its entirety.

In some embodiments, the polymer is a polypeptide. As used herein, the term “polypeptide” means two or more amino acids or residues linked by a peptide bond. The terms “polypeptide” and “protein” are used interchangeably herein and include oligopeptides and peptides. An “amino acid,” “residue,” or “peptide” refers to any of the twenty standard structural units of proteins as known in the art, which include imino acids, such as proline and hydroxyproline. For instance, in some embodiments, the term “residue” when referring to amino acids in a polypeptide is intended to mean a radical derived from the corresponding amino acid by eliminating the hydroxyl of the carboxy group and one hydrogen of the amino group. For example, the terms Gln, Ala, Gly, Ile, Arg, Asp, Phe, Ser, Leu, Cys, Asn, and Tyr represent the residues of L-glutamine, L-alanine, glycine, L-isoleucine, L-arginine, L-aspartic acid, L-phenylalanine, L-serine, L-leucine, L-cysteine, L-asparagine, and L-tyrosine, respectively. The designation of an amino acid isomer may include D, L, R and S. The definition of amino acid includes nonnatural amino acids. Thus, selenocysteine, pyrrolysine, lanthionine, 2-aminoisobutyric acid, gamma-aminobutyric acid, dehydroalanine, ornithine, citrulline and homocysteine are all considered amino acids. Other variants or analogs of the amino acids are known in the art. Thus, a polypeptide may include synthetic peptidomimetic structures such as peptoids. See, Simon et al., 1992, Proceedings of the National Academy of Sciences USA, 89, 9367, which is hereby incorporated by reference herein in its entirety. See also, Chin et al., 2003, Science 301, 964; and Chin et al., 2003, Chemistry & Biology 10, 511, each of which is incorporated by reference herein in its entirety.

In some embodiments, a polypeptide or protein is not limited to a minimum length. Thus, peptides, oligopeptides, dimers, multimers, and the like, are included within the definition. Both full-length proteins and fragments thereof are encompassed by the definition.

The polypeptides evaluated in accordance with some embodiments of the disclosed systems and methods may also have any number of post-expression and/or posttranslational modifications. Thus, a polypeptide includes those that are modified by acylation, alkylation, amidation, biotinylation, formylation, γ-carboxylation, glutamylation, glycosylation, glycylation, hydroxylation, iodination, isoprenylation, lipoylation, cofactor addition (for example, of a heme, flavin, metal, etc.), addition of nucleosides and their derivatives, oxidation, reduction, pegylation, phosphatidylinositol addition, phosphopantetheinylation, phosphorylation, pyroglutamate formation, racemization, addition of amino acids by tRNA (for example, arginylation), sulfation, selenoylation, ISGylation, SUMOylation, ubiquitination, chemical modifications (for example, citrullination and deamidation), and treatment with other enzymes (for example, proteases, phosphotases and kinases). Other types of posttranslational modifications are known in the art and are also included.

In some embodiments, the polymer is an organometallic complex. An organometallic complex is chemical compound containing bonds between carbon and metal. In some instances, organometallic compounds are distinguished by the prefix “organo-,” e.g., organopalladium compounds. Examples of such organometallic compounds include all Gilman reagents, which contain lithium and copper. Tetracarbonyl nickel, and ferrocene are examples of organometallic compounds containing transition metals. Other examples include organomagnesium compounds like iodo(methyl)magnesium MeMgI, diethylmagnesium (Et₂Mg), and all Grignard reagents; organolithium compounds such as n-butyllithium (n-BuLi), organozine compounds such as diethylzine (Et₂Zn) and chloro(ethoxycarbonylmethyl)zinc (ClZₙCH₂C(═O)OEt); and organocopper compounds such as lithium dimethylcuprate (Li⁺[CuMe₂]⁻). In addition to the traditional metals, lanthanides, actinides, and semimetals, elements such as boron, silicon, arsenic, and selenium are considered form organometallic compounds, e.g., organoborane compounds such as triethylborane (Et₃B).

In some embodiments, the polymer is a surfactant. Surfactants are compounds that lower the surface tension of a liquid, the interfacial tension between two liquids, or that between a liquid and a solid. Surfactants may act as detergents, wetting agents, emulsifiers, foaming agents, and dispersants. Surfactants are usually organic compounds that are amphiphilic, meaning they contain both hydrophobic groups (their tails) and hydrophilic groups (their heads). Therefore, a surfactant molecular system contains both a water insoluble (or oil soluble) component and a water soluble component. Surfactant molecules will diffuse in water and adsorb at interfaces between air and water or at the interface between oil and water, in the case where water is mixed with oil. The insoluble hydrophobic group may extend out of the bulk water phase, into the air or into the oil phase, while the water soluble head group remains in the water phase. This alignment of surfactant molecules at the surface modifies the surface properties of water at the water/air or water/oil interface.

Examples of ionic surfactants include ionic surfactants such as anionic, cationic, or zwitterionic (ampoteric) surfactants. Anionic surfactants include (i) sulfates such as alkyl sulfates (e.g., ammonium lauryl sulfate, sodium lauryl sulfate), alkyl ether sulfates (e.g., sodium laureth sulfate, sodium myreth sulfate), (ii) sulfonates such as docusates (e.g., dioctyl sodium sulfosuccinate), sulfonate fluorosurfactants (e.g., perfluorooctanesulfonate and perfluorobutanesulfonate), and alkyl benzene sulfonates, (iii) phosphates such as alkyl aryl ether phosphate and alkyl ether phosphate, and (iv) carboxylates such as alkyl carboxylates (e.g., fatty acid salts (soaps) and sodium stearate), sodium lauroyl sarcosinate, and carboxylate fluorosurfactants (e.g., perfluorononanoate, perfluorooctanoate, etc.). Cationic surfactants include pH-dependent primary, secondary, or tertiary amines and permanently charged quaternary ammonium cations. Examples of quaternary ammonium cations include alkyltrimethylammonium salts (e.g., cetyl trimethylammonium bromide, cetyl trimethylammonium chloride), cetylpyridinium chloride (CPC), benzalkonium chloride (BAC), benzethonium chloride (BZT), 5-bromo-5-nitro-1,3-dioxane, dimethyldioctadecylammonium chloride, and dioctadecyldimethylammonium bromide (DODAB). Zwitterionic surfactants include sulfonates such as CHAPS (3-[(3-Cholamidopropyl)dimethylammonio]-1-propanesulfonate) and sultaines such as cocamidopropyl hydroxysultaine. Zwitterionic surfactants also include carboxylates and phosphates.

Nonionic surfactants include fatty alcohols such as cetyl alcohol, stearyl alcohol, cetostearyl alcohol, and oleyl alcohol. Nonionic surfactants also include polyoxyethylene glycol alkyl ethers (e.g., octaethylene glycol monododecyl ether, pentaethylene glycol monododecyl ether), polyoxypropylene glycol alkyl ethers, glucoside alkyl ethers (decyl glucoside, lauryl glucoside, octyl glucoside, etc.), polyoxyethylene glycol octylphenol ethers (C₈H₁₇—(C₆H₄)—(O—C₂H₄)₁₋₂₅—OH), polyoxyethylene glycol alkylphenol ethers (C₉H₁₉—(C₆H₄)—(O—C₂H₄)₁₋₂₅—OH, glycerol alkyl esters (e.g., glyceryl laurate), polyoxyethylene glycol sorbitan alkyl esters, sorbitan alkyl esters, cocamide MEA, cocamide DEA, dodecyldimethylamine oxide block copolymers of polyethylene glycol and polypropylene glycol (poloxamers), and polyethoxylated tallow amine. In some embodiments, the polymer under study is a reverse micelle, or liposome.

In some embodiments, the polymer is a fullerene. A fullerene is any molecular system composed entirely of carbon, in the form of a hollow sphere, ellipsoid or tube. Spherical fullerenes are also called buckyballs, and they resemble the balls used in association football. Cylindrical ones are called carbon nanotubes or bucky tubes. Fullerenes are similar in structure to graphite, which is composed of stacked graphene sheets of linked hexagonal rings; but they may also contain pentagonal (or sometimes heptagonal) rings.

In some embodiments, the set of M three-dimensional coordinates {x₁, . . . , x_{M}} for the polymer are obtained by x-ray crystallography, nuclear magnetic resonance spectroscopic techniques, or electron microscopy. In some embodiments, the set of M three-dimensional coordinates {x₁, . . . , x_{M}} is obtained by modeling (e.g., molecular dynamics simulations).

In some embodiments, the polymer includes two different types of polymers, such as a nucleic acid bound to a polypeptide. In some embodiments, the polymer includes two polypeptides bound to each other. In some embodiments, the polymer under study includes one or more metal ions (e.g., a metalloproteinase with one or more zinc atoms) and/or is bound to one or more organic small molecules (e.g., an inhibitor). In such instances, the metal ions and or the organic small molecules may be represented as one or more additional particles pᵢ in the set of {p₁, . . . , p_{K}} particles representing the native polymer.

As used herein the term “untrained model” (e.g., “untrained classifier” and/or “untrained neural network”) refers to a machine learning model or algorithm such as a neural network that has not been trained on a target dataset. In some embodiments, “training a model” refers to the process of training an untrained or partially trained model. For instance, consider the case of a plurality of structural input features (e.g., residue feature sets) discussed below. The respective structural input features are applied as collective input to an untrained model, in conjunction with polymer sequences for the respective polymers represented by the plurality of structural input features (hereinafter “training dataset”) to train the untrained model on polymer sequences, including mutations, that satisfy the physical objectives measured by the structural features, thereby obtaining a trained model. Moreover, it will be appreciated that the term “untrained model” does not exclude the possibility that transfer learning techniques are used in such training of the untrained model. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained model described above is provided with additional data over and beyond that of the primary training dataset. That is, in non-limiting examples of transfer learning embodiments, the untrained model receives (i) structural input features and the polymer sequences of each of the respective polymers represented by the plurality of structural input features (“training dataset”) and (ii) additional data. Typically, this additional data is in the form of coefficients (e.g., regression coefficients) that were learned from another, auxiliary training dataset.

More specifically, in some embodiments, the target training dataset is in the form of a first two-dimensional matrix, with one axis representing polymers, and the other axis representing some property of respective polymers, such as residue features. Application of pattern classification techniques to the auxiliary training dataset yields a second two-dimensional matrix, where one axis is the learned coefficients, and the other axis is the property of respective polymers in the auxiliary training dataset. Matrix multiplication of the first and second matrices by their common dimension (e.g., residue feature data) yields a third matrix of auxiliary data that can be applied, in addition to the first matrix to the untrained model. One reason it might be useful to train the untrained model using this additional information from an auxiliary training dataset is a paucity of training objects (e.g., polymers) in one or more categories in the target dataset. This is a particular issue for many healthcare datasets, where there may not be a large number of polymers of a particular category with known feature data (e.g., for a particular disease or a particular stage of a given disease). Making use of as much of the available data as possible can increase the accuracy of classifications and thus improve outputs. Thus, in the case where an auxiliary training dataset is used to train an untrained model beyond just the target training dataset, the auxiliary training dataset is subjected to classification techniques (e.g., principal component analysis followed by logistic regression) to learn coefficients (e.g., regression coefficients) that discriminate amino acid identities based on the auxiliary training dataset. Such coefficients can be multiplied against a first instance of the target training dataset and inputted into the untrained model in conjunction with the target training dataset as collective input, in conjunction with the target training dataset. As one of skill in the art will appreciate, such transfer learning can be applied with or without any form of dimension reduction technique on the auxiliary training dataset or the target training dataset. For instance, the auxiliary training dataset (from which coefficients are learned and used as input to the untrained model in addition to the target training dataset) can be subjected to a dimension reduction technique prior to regression (or other form of label based classification) to learn the coefficients that are applied to the target training dataset. Alternatively, no dimension reduction other than regression or some other form of pattern classification is used in some embodiments to learn such coefficients from the auxiliary training dataset prior to applying the coefficients to an instance of the target training dataset (e.g., through matrix multiplication where one matrix is the coefficients learned from the auxiliary training dataset and the second matrix is an instance of the target training dataset). Moreover, in some embodiments, rather than applying the coefficients learned from the auxiliary training dataset to the target training dataset, such coefficients are applied (e.g., by matrix multiplication based on a common axis of residue feature data) to the residue feature data that was used as a basis for forming the target training set as disclosed herein.

Moreover, while a description of a single auxiliary training dataset has been disclosed, it will be appreciated that there is no limit on the number of auxiliary training datasets that may be used to complement the primary (e.g., target) training dataset in training the untrained model in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning may be used in such embodiments. For instance, consider the case where there is a first auxiliary training dataset and a second auxiliary training dataset in addition to the primary training dataset. The coefficients learned from the first auxiliary training dataset (by application of a model such as regression to the first auxiliary training dataset) may be applied to the second auxiliary training dataset using transfer learning techniques (e.g., the above described two-dimensional matrix multiplication), which in turn may result in a trained intermediate model whose coefficients are then applied to the primary training dataset and this, in conjunction with the primary training dataset itself, is applied to the untrained classifier. Alternatively, a first set of coefficients learned from the first auxiliary training dataset (by application of a model such as regression to the first auxiliary training dataset) and a second set of coefficients learned from the second auxiliary training dataset (by application of a model such as regression to the second auxiliary training dataset) may each individually be applied to a separate instance of the primary training dataset (e.g., by separate independent matrix multiplications) and both such applications of the coefficients to separate instances of the primary training dataset in conjunction with the primary training dataset itself (or some reduced form of the primary training dataset such as principal components or regression coefficients learned from the primary training set) may then be applied to the untrained classifier in order to train the untrained classifier. In either example, knowledge regarding polymer sequences derived from the first and second auxiliary training datasets is used, in conjunction with the polymer sequence-labeled primary training dataset), to train the untrained model.

As used herein, the term “vector” is an enumerated list of elements, such as an array of elements, where each element has an assigned meaning. As such, the term “vector” as used in the present disclosure is interchangeable with the term “tensor.” As an example, a twenty element probability vector for a plurality of amino acids comprises a predetermined element in the vector for each one of the 20 amino acids, where each predetermined element is a respective probability for the respective amino acid. For ease of presentation, in some instances a vector may be described as being one-dimensional. However, the present disclosure is not so limited. A vector of any dimension may be used in the present disclosure provided that a description of what each element in the vector represents is defined (e.g., that element 1 represents a probability of a first amino acid in a plurality of amino acids, etc.).

The following provides system and methods that make use of the processes described above for predicting polymer sequences using a neural network.

### Exemplary System Embodiments.

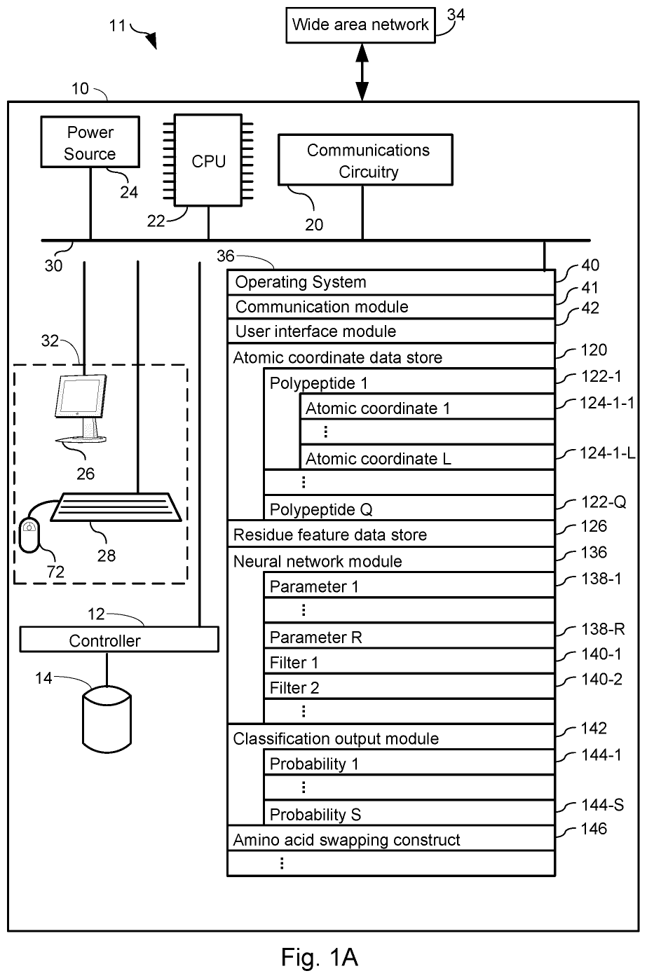

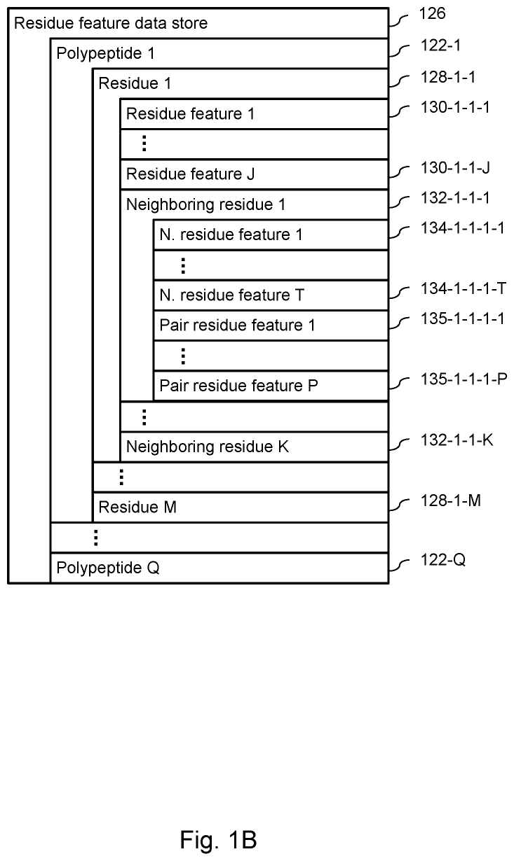

FIGS. 1A and 1B is a block diagram illustrating a system 11 in accordance with one such embodiment. The computer 10 typically includes one or more processing units (CPU's, sometimes called processors) 22 for executing programs (e.g., programs stored in memory 36), one or more network or other communications interfaces 20, memory 36, a user interface 32, which includes one or more input devices (such as a keyboard 28, mouse 72, touch screen, keypads, etc.) and one or more output devices such as a display device 26, one or more magnetic disk storage and/or persistent devices 14 optionally accessed by one or more controllers 12, and one or more communication buses 30 for interconnecting these components, and a power supply 24 for powering the aforementioned components. The communication buses 30 may include circuitry (sometimes called a chipset) that interconnects and controls communications between system components.

Memory 36 includes high-speed random access memory, such as DRAM, SRAM, DDR RAM or other random access solid state memory devices; and typically includes non-volatile memory, such as one or more magnetic disk storage devices, optical disk storage devices, flash memory devices, or other non-volatile solid state storage devices. Memory 36 optionally includes one or more storage devices remotely located from the CPU(s) 22. Memory 36, or alternately the non-volatile memory device(s) within memory 36, comprises a non-transitory computer readable storage medium. In some embodiments, memory 36 or the computer readable storage medium of memory 36 stores the following programs, modules and data structures, or a subset thereof:
an optional operating system 40 that includes procedures for handling various basic system services and for performing hardware dependent tasks;
an optional communication module 41 that is used for connecting the computer 10 to other computers via the one or more communication interfaces 20 (wired or wireless) and one or more communication networks 34, such as the Internet, other wide area networks, local area networks, metropolitan area networks, and so on;
an optional user interface module 42 that receives commands from the user via the input devices 28, 72, etc. and generates user interface objects in the display device 26;
an atomic coordinate data store 120 including a plurality of atomic coordinates 124 (e.g., 124-1-1, . . . 124-1-L) of a polypeptide 122 (e.g., in a plurality of polypeptides 122-1, . . . 122-Q);
a residue feature data store 126 including, for a respective polypeptide 122, for each respective residue 128 in a plurality of residues for the polypeptide (e.g., 128-1-1, . . . 128-1-M), a corresponding residue feature set obtained using the plurality of atomic coordinates 124 for the polypeptide, the corresponding residue feature set comprising:
   one or more residue features 130 for the respective residue (e.g., 130-1-1-1, . . . 130-1-1-J) and, for each respective neighboring residue 132 in a plurality of neighboring residues (e.g., 132-1-1-1, . . . 132-1-1-K), one or more neighboring residue features 134 (e.g., 134-1-1-1-1, . . . 134-1-1-1-T) and one or more pair residue features 135 (e.g., 135-1-1-1-1, . . . 135-1-1-1-P) for the respective residue and the respective neighboring residue, where each respective neighboring residue 132 has a C carbon that is within a nearest neighbor cutoff of the C carbon of the respective residue 128, and where a respective residue feature 130 and/or a respective neighboring residue feature 134 in the corresponding residue feature set includes one or more of:
      an indication of the secondary structure of the respective residue 128 and/or neighboring residue 132 encoded as one of helix, sheet and loop,
      a relative solvent accessible surface area of the C_{α}, N, C, and O backbone atoms of the respective residue 128 and/or neighboring residue 132,
      cosine and sine values of the backbone dihedrals ϕ, ψ and ω of the respective residue 128 and/or neighboring residue 132,
      a C_{α} to C_{α} distance of each neighboring residue 132 having a C_{α} carbon within a threshold distance of the C_{α} carbon of the respective residue 128, and
      an orientation and position of a backbone of each neighboring residue 132 relative to the backbone residue segment of the respective residue 128 in a reference frame centered on C atom of the respective residue 128;
a neural network module 136 that accepts, as input, the residue feature set for a respective identified residue 128 in the plurality of residues for the respective polypeptide 122 and that comprises a plurality of parameters 138 (e.g., 138-1, . . . 138-R) and, optionally, at least a first filter 140-1 and a second filter 140-2;
a classification output module 142 that stores a plurality of probabilities 144 (e.g., 144-1, . . . 144-S), including a probability for each respective naturally occurring amino acid; and
an optional amino acid swapping construct that obtains a respective swap amino acid identity for a respective residue 128 based on a draw from the corresponding plurality of probabilities 144 and, when the respective swap amino acid identity of the respective residue changes the identity of the respective residue, updates each corresponding residue feature 130 and/or 134 in the residue feature data store 126 affected by the change in amino acid identity.

In some embodiments, the programs or modules identified in FIGS. 1A and 1B correspond to sets of instructions for performing a function described above. The sets of instructions can be executed by one or more processors (e.g., the CPUs 22). The above identified modules or programs (e.g., sets of instructions) need not be implemented as separate software programs, procedures or modules, and thus various subsets of these programs or modules may be combined or otherwise re-arranged in various embodiments. In some embodiments, memory 36 stores a subset of the modules and data structures identified above. Furthermore, memory 36 may store additional modules and data structures not described above.

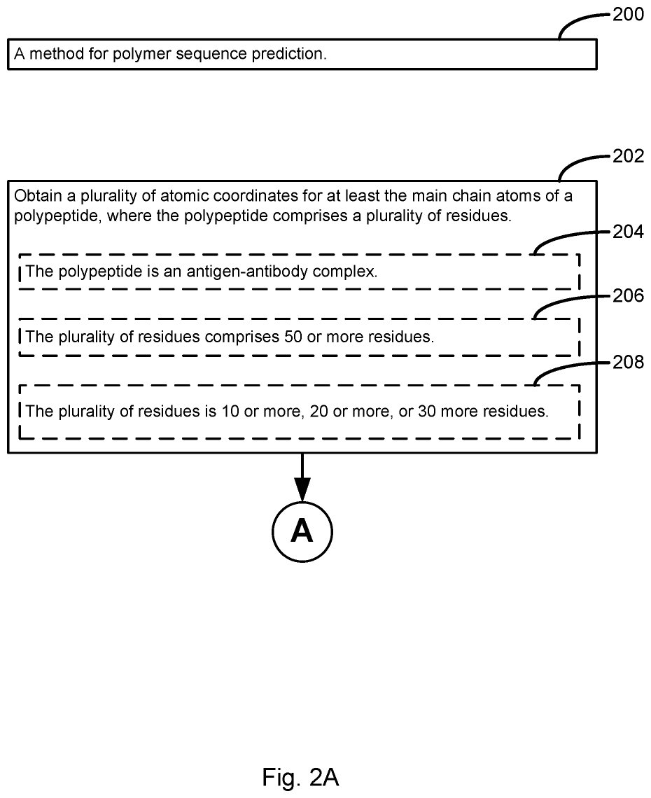

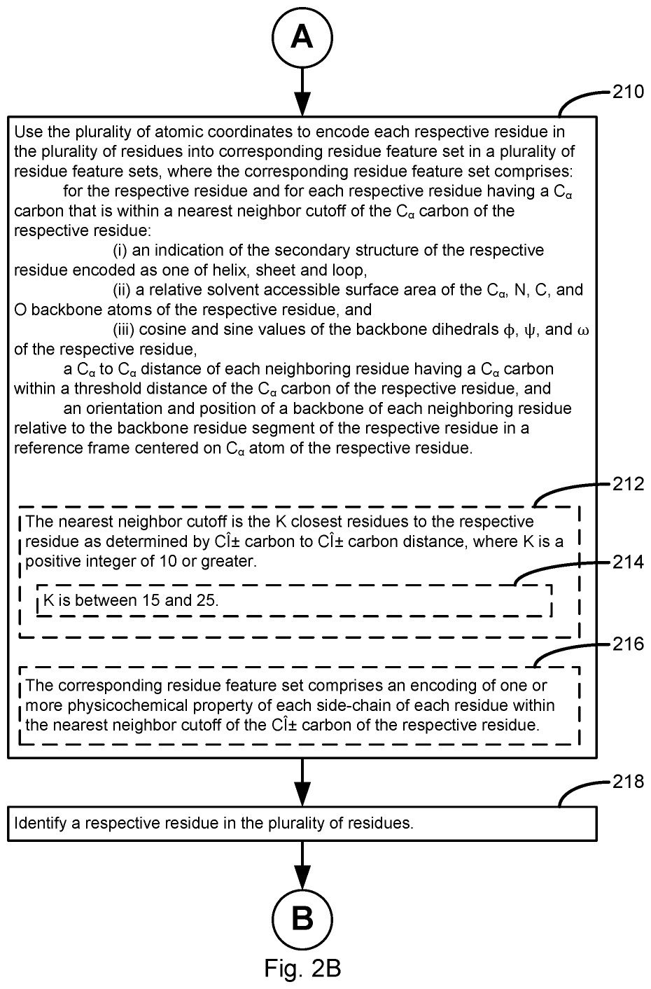

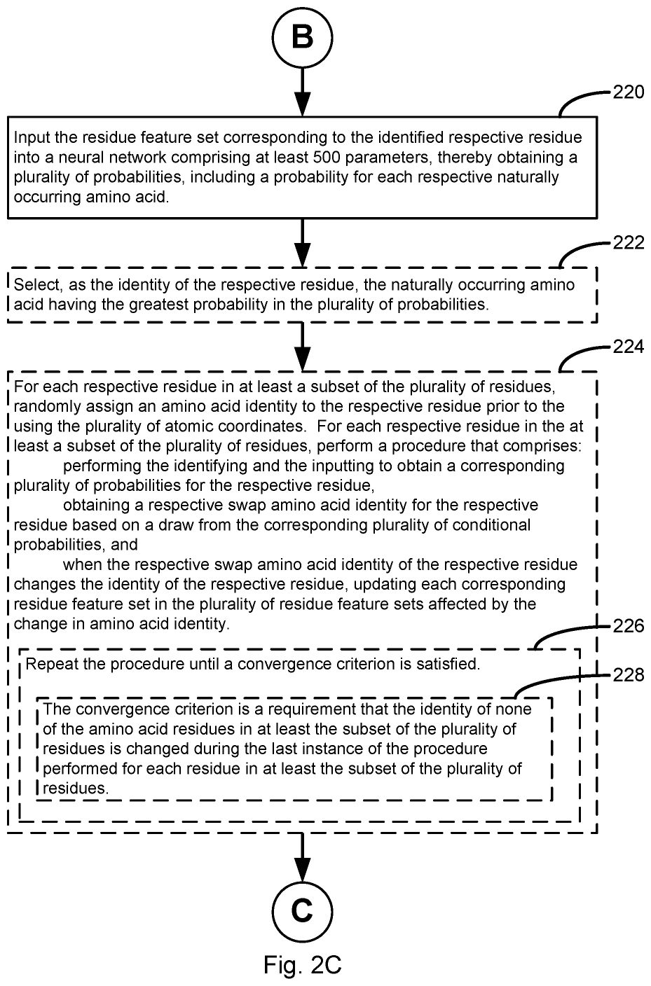

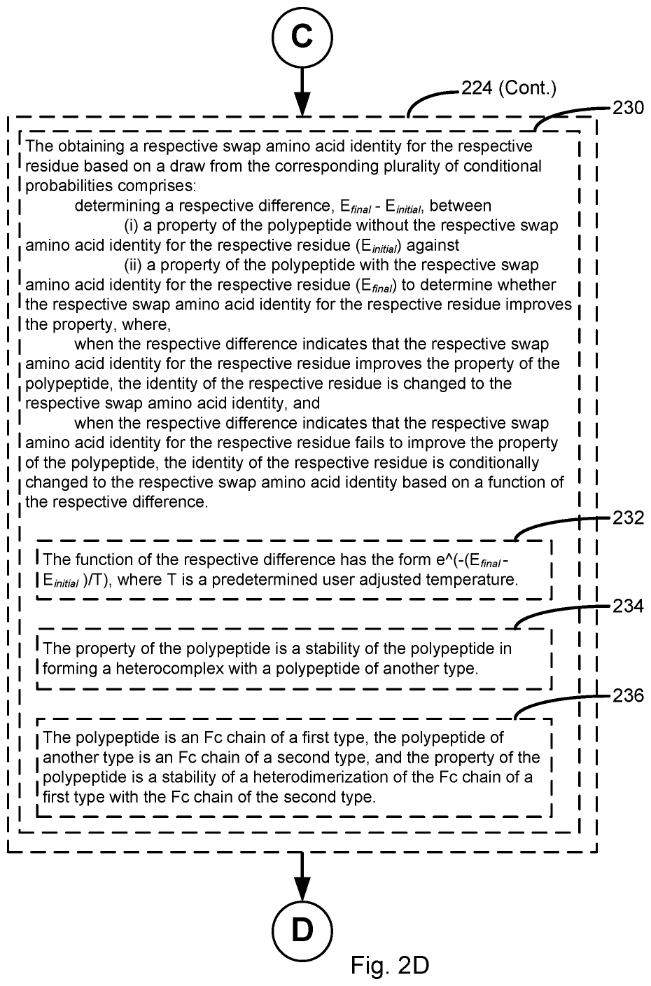

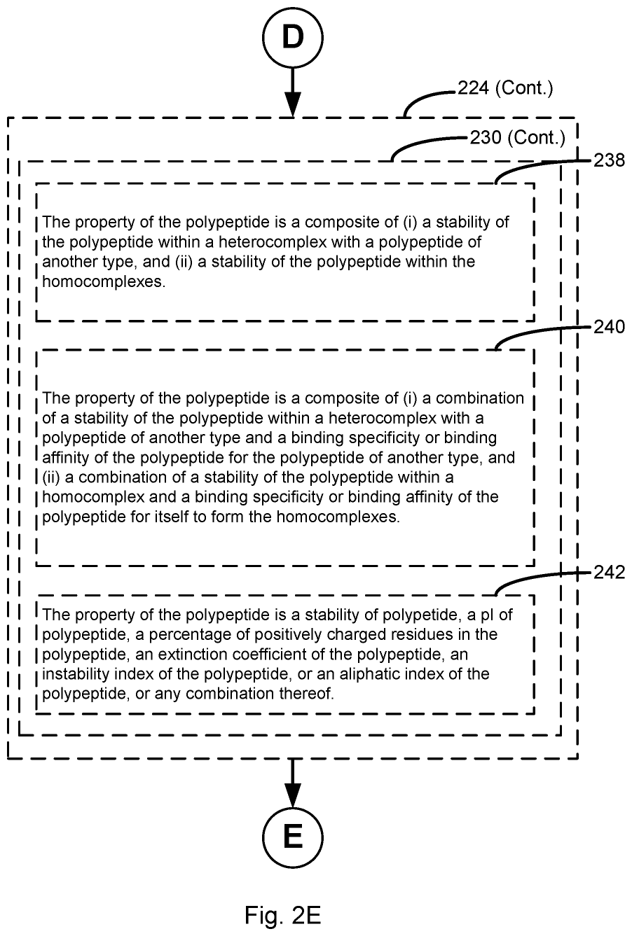

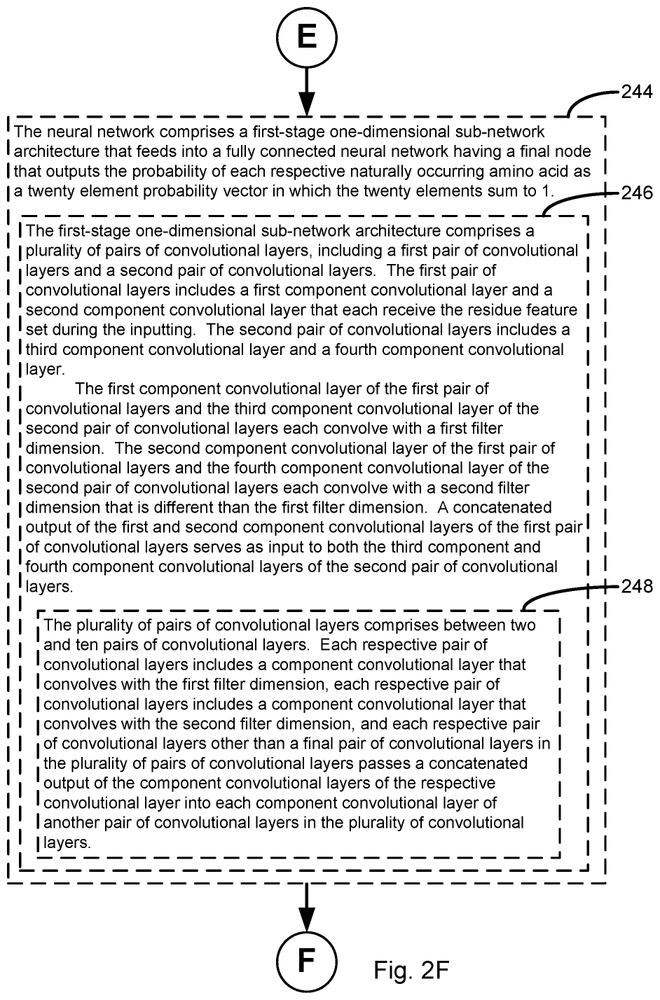

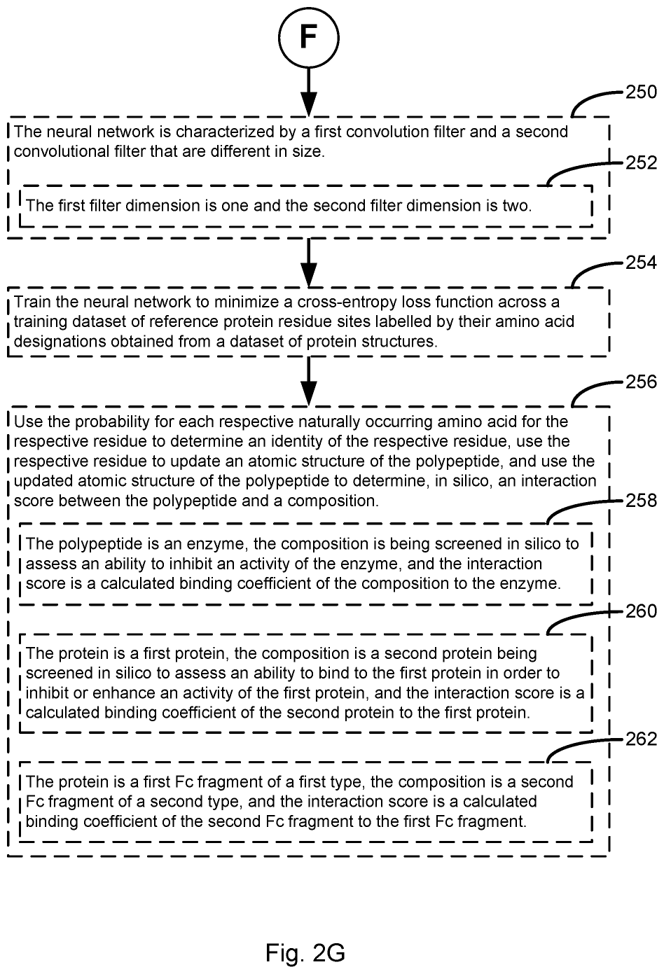

Now that a system in accordance with the systems and methods of the present disclosure has been described, attention turns to FIGS. 2A, 2B, 2C, 2D, 2E, 2F, and 2G which illustrate an exemplary method for polymer sequence prediction 200 in accordance with the present disclosure.

### Exemplary Embodiments for Polymer Sequence Prediction.

In one aspect of the present disclosure, the method 200 is performed at a computer system comprising one or more processors and memory addressable by the one or more processors, the memory storing at least one program for executing the method 200 by the one or more processors.

Accordingly, referring to Block 202, the method 200 comprises obtaining a plurality of atomic coordinates for at least the main chain atoms of a polypeptide, where the polypeptide comprises a plurality of residues.

### Polypeptides and Atomic Coordinates.

Referring to Block 202, the method comprises obtaining a plurality of atomic coordinates 124 for at least the main chain atoms of a polypeptide 122, where the polypeptide comprises a plurality of residues 128.

In some embodiments, the polypeptide is a protein. For instance, in some embodiments, the polypeptide is any of the embodiments for polymers disclosed herein (see, for example, Definitions: Polymers, above).

In some embodiments, the polypeptide is complexed with one or more additional polypeptides. For instance, in some embodiments, the polypeptide has a first type and is in a heterocomplex with another polypeptide of another type. In some embodiments, the polypeptide is in a homodimeric complex with another polypeptide of the same type.

In some embodiments, the polypeptide is an Fc chain. For instance, in some embodiments, the polypeptide is an Fc chain of a first type and is in a heterocomplex with a polypeptide that is an Fc chain of a second type. In some embodiments, the polypeptide is an antigen-binding fragment (Fab).

Referring to Block 204, in some embodiments, the polypeptide is an antigen-antibody complex.

In some embodiments, the polypeptide comprises a protein sequence and/or structure obtained from a respective plurality of protein sequences and/or structures (e.g., search space 302).

In some embodiments, the polypeptide comprises a sequence and/or structure obtained from a database. Suitable databases for protein sequences and structures include, but are not limited to, protein sequence databases (e.g., DisProt, InterPro, MobiDB, neXtProt, Pfam, PRINTS, PROSITE, the Protein Information Resource, SUPERFAMILY, Swiss-Prot, NCBI, etc.), protein structure databases (e.g., the Protein Data Bank (PDB), the Structural Classification of Proteins (SCOP), the Protein Structure Classification (CATH) Database, Orientations of Proteins in Membranes (OPM) database, etc.), protein model databases (e.g., ModBase, Similarity Matrix of Proteins (SIMAP), Swiss-model, AAindex, etc.), protein-protein and molecular interaction databases (e.g., BioGRID, RNA-binding protein database, Database of Interacting Proteins, IntAct, etc.), protein expression databases (e.g., Human Protein Atlas, etc.), and/or physicochemical databases (e.g., the Amino acid Physico-chemical properties Database (APD)). See, for example, Mathura and Kolippakkam, “APDbase: Amino acid Physico-chemical properties Database,” Bioinformation 1(1): 2-4 (2005), which is hereby incorporated herein by reference in its entirety. In some embodiments, the polypeptide is obtained from any suitable database disclosed herein, as will be apparent to one skilled in the art.

In some embodiments, the method is performed for a respective polypeptide in a plurality of polypeptides. In some embodiments, the method is performed for each respective polypeptide in a plurality of polypeptides. In some embodiments, the plurality of polypeptides is at least 2, at least 3, at least 4, at least 5, at least 10, at least 15, at least 20, at least 30, at least 50, or at least 100 polypeptides. In some embodiments, the plurality of polypeptides is from 5 to 1000 polypeptides.

Referring to Block 206, in some embodiments, the plurality of residues comprises 50 or more residues. Referring to Block 208, the plurality of residues is 10 or more, 20 or more, or 30 more residues. In some instances, the plurality of residues comprises between 2 and 5,000 residues, between 20 and 50,000 residues, more than 30 residues, more than 50 residues, or more than 100 residues. In some instances, the plurality of residues comprises ten or more, twenty or more, thirty or more, fifty or more, one hundred or more, one thousand or more, or one hundred thousand or more residues. In some embodiments, the plurality of residues comprises no more than 200,000, no more than 100,000, no more than 10,000, no more than 1000, or no more than 500 residues. In some embodiments, the plurality of residues comprises from 100 to 1000, from 50 to 100,000, or from 10 to 5000 residues. In some embodiments, the plurality of residues falls within another range starting no lower than 2 residues and ending no higher than 200,000 residues.

In some embodiments, the plurality of residues comprises all or a subset of the total residues in the polypeptide. In some embodiments, the plurality of residues is less than the total number of residues in the polypeptide.

In some embodiments, the amino acid identity of one or more respective residues in the plurality of residues is known. For example, in some embodiments, a respective residue in the plurality of residues has a wild-type amino acid identity (e.g., of a native form of the polypeptide). In some embodiments, a respective residue in the plurality of residues is a mutated residue of an engineered form of the polypeptide, (e.g., having a non-wild-type amino acid identity).

In some instances, a residue in the polypeptide comprises two or more atoms, three or more atoms, four or more atoms, five or more atoms, six or more atoms, seven or more atoms, eight or more atoms, nine or more atoms or ten or more atoms. In some instances, the polypeptide has a molecular weight of 100 Daltons or more, 200 Daltons or more, 300 Daltons or more, 500 Daltons or more, 1000 Daltons or more, 5000 Daltons or more, 10,000 Daltons or more, 50,000 Daltons or more or 100,000 Daltons or more.

In some embodiments, the obtaining the plurality of atomic coordinates for at least the main chain atoms of a polypeptide comprises obtaining atomic coordinates for only the main chain (e.g., backbone) atoms of the polypeptide. In some embodiments, the obtaining the plurality of atomic coordinates for at least the main chain atoms of a polypeptide comprises obtaining atomic coordinates for the main chain (e.g., backbone) atoms and one or more side chains of the polypeptide. In some embodiments, the obtaining the plurality of atomic coordinates comprises obtaining atomic coordinates for only the side chain atoms of the polypeptide. In some embodiments, the obtaining the plurality of atomic coordinates for at least the main chain atoms of a polypeptide comprises obtaining atomic coordinates for at least one or more atoms in the main chain of the polypeptide and/or one or more atoms in a respective one or more side chains of the polypeptide. In some embodiments, the obtaining the plurality of atomic coordinates for at least the main chain atoms of a polypeptide comprises obtaining atomic coordinates for all of the atoms in the polypeptide.

In some embodiments, the plurality of atomic coordinates is represented as a set of {x₁, . . . , x_{N}} coordinates. In some embodiments, each coordinate xᵢ in the set of is that of a heavy atom in the protein. In some embodiments, the set {x₁, . . . , x_{N}} further includes the coordinates of hydrogen atoms in the polypeptide.

In some instances, the plurality of atomic coordinates {x₁, . . . , x_{N}} are obtained by x-ray crystallography, nuclear magnetic resonance spectroscopic techniques, and/or electron microscopy. In some instances, the plurality of atomic coordinates {x₁, . . . , x_{N}} is obtained by modeling (e.g., molecular dynamics simulations). In some instances, each respective atomic coordinate in the plurality of atomic coordinates {x₁, . . . , x_{N}} is a coordinate in three dimensional space (e.g., x, y z).

In some embodiments, the polypeptide is subjected to one or more selection criteria, prior to obtaining the plurality of atomic coordinates, where, when the polypeptide satisfies all or a subset of the selection criteria, the polypeptide is deemed suitable for further processing and downstream consumption by the method (e.g., generation of residue features for input to a neural network).

For example, in some embodiments, the one or more selection criteria includes a determination that the structure of the polypeptide is obtained using x-ray crystallography, nuclear magnetic resonance spectroscopic techniques, and/or electron microscopy. In some embodiments, the one or more selection criteria includes a determination that the resolution of the structure of the polypeptide is above a threshold resolution. For example, in some such embodiments, the threshold resolution is at least (e.g., better than) 10 Å, at least 5 Å, at least 4 Å, at least 3 Å, at least 2 Å, or at least 1 Å. In some embodiments, the one or more selection criteria includes a determination that the main chain (e.g., backbone) length of the polypeptide is longer than a threshold length. For instance, in some such embodiments, the threshold length is at least 10 residues, at least 15 residues, at least 20 residues, at least 50 residues, or at least 100 residues. In some embodiments, the one or more selection criteria includes a determination that the structure of the polypeptide does not have any DNA/RNA molecules. In some embodiments, the one or more selection criteria includes a determination that the polypeptide is not a membrane protein. In still other embodiments, the one or more selection criteria includes a determination that the protein structure of the polypeptide does not comprise D-amino acids.

In some embodiments, the polypeptide is further processed prior to further operation of the method (e.g., generation of residue features for input to a neural network). In some such embodiments, the processing comprises removing one or more residues from the polypeptide structure. For example, in some such embodiments, the polypeptide structure is processed to remove non-protein residues such as water, ion, and ligand. In some embodiments, the polypeptide structure is processed to remove residues comprising atoms with an occupancy of <1 and/or residues in which one or more backbone atoms are missing. In some embodiments, the processing comprises removing one or more subunits from the polypeptide structure. For example, in some such embodiments, the polypeptide structure is processed to collapse one or more repeated subunits (e.g., if a structure contains several identical subunits, all but one of the identical subunits are removed). In some embodiments, the processing comprises performing a translation and/or rotation of one or more residues in the polypeptide structure. For instance, in some embodiments, the polypeptide structure is processed by translating and/or orientating one or more residues such that the Cα, N, and C atoms of the respective one or more residues are located at the origin, the −x axis, and the z=0 plane, respectively.

Additional methods for obtaining atomic coordinates, including obtaining and processing polypeptide structures, suitable for use in the present disclosure are further described in, Wang et al., “Computational Protein Design with Deep Learning Neural Networks.” Nature. Sci. Rep. 8, 6349 (2018), which is hereby incorporated herein by reference in its entirety.

### Residue Feature Sets.

Referring to Block 210, the method further includes using the plurality of atomic coordinates 124 to encode each respective residue 128 in the plurality of residues into a corresponding residue feature set 312 in a plurality of residue feature sets.

The corresponding residue feature set 312 comprises, for the respective residue and for each respective residue having a C_{α} carbon that is within a nearest neighbor cutoff of the C_{α} carbon of the respective residue, (i) an indication of the secondary structure of the respective residue encoded as one of helix, sheet and loop, (ii) a relative solvent accessible surface area of the C_{α}, N, C, and O backbone atoms of the respective residue, and (iii) cosine and sine values of the backbone dihedrals ϕ, ψ and ω of the respective residue.

The corresponding residue feature set further includes a C_{α} to C_{α} distance of each neighboring residue having a C_{α} carbon within a threshold distance of the C_{α} carbon of the respective residue, and an orientation and position of a backbone of each neighboring residue relative to the backbone residue segment of the respective residue in a reference frame centered on C_{α} atom of the respective residue.

In other words, in some embodiments, a corresponding residue feature set 312 for a respective residue in the plurality of residues includes, e.g., backbone features or, alternatively, backbone and side chain features. As described above, in some implementations, backbone features refer to features that are computed using only the backbone atoms of the polypeptide, while side chain features refer to features that are computed using the side chain atoms of the polypeptide. Moreover, as described above, these features collectively describe the local protein environment neighboring (e.g., surrounding) a respective residue (e.g., a given target residue). Such features include, but are not limited to, features specific to a respective target residue 130, features specific to a neighboring residue of a respective target residue 134, and/or features specific to a pair of the respective target residue and a neighboring residue 135. In some embodiments, the neighboring environment is defined, for instance, by the K (e.g., a positive integer) nearest neighboring residues 132 to the target residue 128 based on the C_{α} to C_{α} residue to residue distance.

In some embodiments, the neighboring environment can thus be represented as a K-star graph centered around the target residue 128 (e.g., the respective residue), with each of K points in the K-star graph indicating a subset of residue features corresponding to a neighboring residue 132 in the K-nearest neighboring residues. This standard frame can be defined so that it is centered on the target residue site, for instance, where the target residue's C_{α} atom is chosen as the origin in this local reference frame.

Thus, referring to Block 212, in some embodiments, the nearest neighbor cutoff is the K closest residues to the respective residue as determined by C_{α} carbon to C_{α} carbon distance, wherein K is a positive integer of 10 or greater. Referring to Block 214, in some embodiments, K is between 15 and 25. In some embodiments, K is at least 2, at least 5, at least 10, at least 15, at least 20, or at least 50. In some embodiments, K is no more than 100, no more than 50, no more than 20, or no more than 10. In some embodiments, K is from 5 to 10, from 2 to 25, from 10 to 50, or from 15 to 22. In some embodiments, K is a positive integer that falls within a range starting no lower than 2 and ending no higher than 100.

In some embodiments, the threshold distance is a predetermined radius, and the neighboring environment is a sphere having a predetermined radius and centered either on a particular atom of the respective residue (e.g., C_{α} carbon in the case of proteins) or the center of mass of the respective residue. In some instances, the predetermined radius is five Angstroms or more, 10 Angstroms or more, or 20 Angstroms or more.

In some instances, a corresponding residue feature set corresponds to two or more respective residues, and the neighboring environment is a concatenation of two or more neighboring environments (e.g., spheres). For example, in some instances, two different residues are identified, and the neighboring environment comprises (i) a first sphere having a predetermined radius that is centered on the Cₐₗₚₕₐ carbon of the first identified residue and (ii) a second sphere having a predetermined radius that is centered on the Cₐₗₚₕₐ carbon of the second identified residue. Depending on how close the two substitutions are, the residues and/or their neighboring environments may or may not overlap. In alternative instances, the corresponding residue feature set corresponding to two or more respective residues, and the neighboring environment is a single contiguous region.

In some embodiments, the two or more respective residues consists of two residues. In some instances, the two or more respective residues consists of three residues. In some instances, the two or more respective residues consists of four residues. In some instances, the two or more respective residues consists of five residues. In some instances, the two or more respective residues comprises more than five residues. In some embodiments, the two or more respective residues are contiguous or noncontiguous within the polypeptide.

In some embodiments, the residue feature set is obtained using a wild-type amino acid identity for the respective residue. In some embodiments, the residue feature set is obtained using a non-wild-type (e.g., mutated) amino acid identity for the respective residue.

In some embodiments, for each respective residue in at least a subset of the plurality of residues, prior to the obtaining the residue feature set, the respective residue is randomly assigned an amino acid identity, and the residue feature set is obtained using the randomly assigned amino identity for the respective residue. Accordingly, in some such embodiments, each respective residue in the plurality of residues is replaced with a different amino acid identity. In some instances, a subset of the plurality of residues are replaced with different amino acid identities. In some instances, none of the residues in the plurality of residues are replaced with different amino acid identities.

In some embodiments, the set of residue features 312 is derived from a respective plurality of protein sequences and/or structures (e.g., search space 302).

In some embodiments, the set of residue features 312 is obtained from atomic coordinates derived from a database.

In some embodiments, the database is the Protein Data Bank (PDB), the Amino acid Physico-chemical properties Database (APD), and/or any suitable database disclosed herein, as will be apparent to one skilled in the art. See, for example, Mathura and Kolippakkam, “APDbase: Amino acid Physico-chemical properties Database,” Bioinformation 1(1): 2-4 (2005), which is hereby incorporated herein by reference in its entirety.

Residue features suitable for use in a corresponding residue feature set include any residue feature known in the art for a respective residue (e.g., a target residue) and/or a neighboring residue of the respective residue. For instance, as described above, referring again to Block 210, a respective residue feature in a corresponding residue feature set can comprise a secondary structure, a crude relative solvent-accessible surface area (SASA), and/or one or more backbone torsion angles (e.g., a dihedral angle of a side chain and/or a main chain) for one or more of the K neighboring residues and the respective residue.

In some embodiments accessible surface area (ASA or SASA), also known as the “accessible surface”, is the surface area of a molecular system that is accessible to a solvent. Measurement of ASA is usually described in units of square Angstroms. ASA is described in Lee & Richards, 1971, J. Mol. Biol. 55(3), 379-400, which is hereby incorporated by reference herein in its entirety. ASA can be calculated, for example, using the “rolling ball” algorithm developed by Shrake & Rupley, 1973, J. Mol. Biol. 79(2): 351-371, which is hereby incorporated by reference herein in its entirety. This algorithm uses a sphere (of solvent) of a particular radius to “probe” the surface of the molecular system. Solvent-excluded surface, also known as the molecular surface or Connolly surface, can be viewed as a cavity in bulk solvent (effectively the inverse of the solvent-accessible surface). It can be calculated in practice via a rolling-ball algorithm developed by Richards, 1977, Annu Rev Biophys Bioeng 6, 151-176 and implemented three-dimensionally by Connolly, 1992, J. Mol. Graphics 11(2), 139-141, each of which is hereby incorporated by reference herein in its entirety.

In some embodiments, a dihedral angle is obtained from a rotamer library, such as optional side chain rotamer database or optional main chain structure database. Examples of such databases are found in, for example, Shapovalov and Dunbrack, 2011, “A smoothed backbone-dependent rotamer library for proteins derived from adaptive kernel density estimates and regressions,” Structure 19, 844-858; Dunbrack and Karplus, 1993, “Backbone-dependent rotamer library for proteins. Application to side chain prediction,” J. Mol. Biol. 230: 543-574; and Lovell et al., 2000, “The Penultimate Rotamer Library,” Proteins: Structure Function and Genetics 40: 389-408, each of which is hereby incorporated by reference herein in its entirety. In some embodiments, the optional side chain rotamer database comprises those referenced in Xiang, 2001, “Extending the Accuracy Limits of Prediction for Side-chain Conformations,” Journal of Molecular Biology 311, p. 421, which is hereby incorporated by reference in its entirety. In some embodiments, the dihedral angle is obtained from a rotamer library on a deterministic, random or pseudo-random basis.

In some embodiments, a respective residue feature in a corresponding residue feature set comprises geometric and/or edge features, including, but not limited to, C_{α} to C_{α} distances between the respective residue and one or more neighboring residues. In some embodiments, a respective residue feature in a corresponding residue feature set comprises an orientation and/or a position of one or more different backbone residue segments of one or more neighboring residues relative to the backbone residue segment of the respective residue (e.g., target residue) in a standardized reference frame.

In some embodiments, a respective residue feature in a corresponding residue feature set comprises an amino acid identity for a respective residue (e.g., a target residue) and/or a neighboring residue.

In some embodiments, a respective residue feature in a corresponding residue feature set comprises a root mean squared distance between a side chain of a first residue in a first three-dimensional structure of the polypeptide and the side chain of the first residue in a second three-dimensional structure of the polypeptide, when the first three-dimensional structure is overlayed on the second three-dimensional structure. In some embodiments, a respective residue feature in a corresponding residue feature set comprises a root mean squared distance between heavy atoms (e.g., non-hydrogen atoms) in a first portion of a first three-dimensional structure of the polypeptide and the corresponding heavy atoms in the portion of a second three-dimensional structure of the polypeptide corresponding to the first portion when the first three-dimensional structure is overlayed on the second three-dimensional structure. In some embodiments, a respective residue feature in a corresponding residue feature set comprises a distance between a first atom and a second atom in the polypeptide, where a first three-dimensional structure of the polypeptide has a first value for this distance and a second three-dimensional structure of the polypeptide has a second value for this distance, such that the first distance deviates from the second distance by the initial value.

In some embodiments, a respective residue feature in a corresponding residue feature set is a rotationally and/or translationally invariant structural feature. In some embodiments, a respective residue feature in a corresponding residue feature set is an embedded topological categorical feature.

In some embodiments, a respective residue feature in a corresponding residue feature set is selected from the group consisting of: number of amino acids (e.g., number of residues in each protein), molecular weight (e.g., molecular weight of the protein), theoretical pI (e.g., pH at which the net charge of the protein is zero (isoelectric point)), amino acid composition (e.g., percentage of each amino acid in the protein), positively charged residue 2 (e.g., percentage of positively charged residues in the protein (lysine and arginine)), positively charged residue 3 (e.g., percentage of positively charged residues in the protein (histidine, lysine, and arginine)), number of atoms (e.g., total number of atoms), carbon (e.g., total number of carbon atoms in the protein sequence), hydrogen (e.g., total number of hydrogen atoms in the protein sequence), nitrogen (e.g., total number of nitrogen atoms in the protein sequence), oxygen (e.g., total number of oxygen atoms in the protein sequence), sulphur (e.g., total number of sulphur atoms in the protein sequence), extinction coefficient all Cys (e.g., amount of light a protein absorbs at a certain wavelength assuming all Cys residues appear as half cysteines), extinction coefficient no Cys (e.g., amount of light a protein absorbs at a certain wavelength assuming no Cys residues appear as half cysteines), instability index (e.g., the stability of the protein), aliphatic index (e.g., the relative volume of the protein occupied by aliphatic side chains), GRAVY (e.g., Grand average of hydropathicity), PPR (e.g., percentage of continuous changes from positively charged residues to positively charged residues), NNR (e.g., percentage of continuous changes from negatively charged residues to negatively charged residues), PNPR (e.g., percentage of continuous changes from positively charged residues to negatively charged residues or from negatively charged residues to positively charged residues), NNRDist(x, y) (e.g., percentage of NNR from x to y (local information)), PPRDist(x, y) (e.g., percentage of PPR from x to y (local information)), PNPRDist(x, y) (e.g., percentage of PNPR from x to y (local information)), negatively charged residues (e.g., percentage of negatively charged residues in the protein), and amino acid pair ratio (e.g., percentage compositions for each of the 400 possible amino acid dipeptides)

In some embodiments, a respective residue feature in a corresponding residue feature set is a physicochemical property selected from the group consisting of charged; polar; aliphatic; aromatic; small; tiny; bulky; hydrophobic; hydrophobic and aromatic; neutral, weakly and hydrophobic; hydrophilic and acidic; hydrophilic and basic; acidic; and polar and uncharged.

In some embodiments, a respective residue feature in a corresponding residue feature set is a physicochemical property selected from the group consisting of steric parameter, polarizability, volume, hydrophobicity, isoelectric point, helix probability, and sheet probability.

In some embodiments, a respective residue feature in a corresponding residue feature set comprises a protein class selected from the group consisting of transport, transcription, translation, gluconate utilization, amino acid biosynthesis, fatty acid metabolism, acetylcholine receptor inhibitor, G-protein coupled receptor, guanine nucleotide-releasing factor, fiber protein, and transmembrane.

In some embodiments, a respective residue feature in a corresponding residue feature set is any suitable protein property or feature known in the art and/or disclosed herein (see, e.g., the section entitled “Sampling methods for amino acid identity selection,” below). For instance, in some embodiments, a respective residue feature in a corresponding residue feature set is any of the protein properties, classes, and/or features disclosed in Lee et al., “Identification of protein functions using a machine-learning approach based on sequence-derived properties,” Proteome Science 2009, 7:27; doi:10.1186/1477-5956-7-27; and Wang et al., “Computational Protein Design with Deep Learning Neural Networks.” Nature. Sci. Rep. 8, 6349 (2018), each of which is hereby incorporated herein by reference in its entirety, and/or any substitutions, modifications, additions, deletions, or combinations thereof, as will be apparent to one skilled in the art.

In some embodiments, one or more features in the residue feature set are encoded, where the encodings represent the feature as a numeric value. In some embodiments, the residue feature set comprises an encoding of amino acid identities for a respective residue and/or neighboring residue of the respective residue. In some embodiments, the residue feature set comprises an encoding of a physicochemical feature. For instance, referring to Block 216, in some embodiments, the corresponding residue feature set comprises an encoding of one or more physicochemical property of each side-chain of each residue within the nearest neighbor cutoff of the C_{α} carbon of the respective residue.

In some embodiments, an encoding of a respective residue feature is generated by a “symmetric neural network” used as an encoder-decoder. Once trained, in some embodiments, the encoder learns to represent a higher order (e.g., 7-D physicochemical properties) vector as a lower order (e.g., 2-D) vector. For instance, in an example embodiment, the training dataset for the encoder-decoder consists of a set of twenty 7-D properties vectors, where each of the twenty amino-acid types has a distinct 7-D properties vector.

In some embodiments, one or more residue features are used as inputs to a neural network model 308. For example, neural networks can be used to classify and predict protein function independent of sequence or structural alignment, based on the one or more residue features obtained from the residue feature sets. See, e.g., Lee et al., “Identification of protein functions using a machine-learning approach based on sequence-derived properties,” Proteome Science 2009, 7:27; doi:10.1186/1477-5956-7-27, which is hereby incorporated herein by reference in its entirety.

In some embodiments, as illustrated in FIG. 3C, a corresponding residue feature set is converted to a matrix 312 (e.g., 312-A, 312-B) for input to the neural network model. In some such embodiments, the matrix is a K×N_{f} matrix 312-A where N_{f} is the number of features pertaining to the target residue site and one of its K neighbors.

### Amino Acid Probabilities.

Referring to Blocks 218 and 220, the method further includes identifying a respective residue 128 in the plurality of residues and inputting the residue feature set 312 corresponding to the identified respective residue 128 into a neural network (e.g., comprising at least 500 parameters 138), thereby obtaining a plurality of probabilities 144, including a probability for each respective naturally occurring amino acid.

In some embodiments, the identifying and inputting is performed for a single respective residue in the plurality of residues.

In some embodiments, the identifying and inputting is performed for a set of residues, where the set of residues comprises two or more residues, three or more residues, four or more residues, five or more residues, ten or more residues, or twenty or more residues. In some embodiments, the identifying and inputting is performed simultaneously for a set of residues. In some instances, the number and identity of residues that are selected for the identifying and inputting is determined on a random, pseudo-random or deterministic basis.

In some embodiments, the output of the model is a probability vector 318 including a respective probability 144 for each of a plurality of amino acid identities possible at a target residue site. In some embodiments, each element of the vector is associated with a particular amino acid identity (e.g., Arginine or Alanine).

In some embodiments, the sum of the plurality of elements (e.g., probabilities) in the probability vector is equal to one. For example, in some such embodiments, the probability vector represents a probability distribution for the 20 amino acids, in which the sum of the 20 elements in the vector is equal to one.

In some embodiments, the output of the neural network model is used to predict amino acid identities at a respective residue (e.g., a target residue site). In other words, in some embodiments, the probability vector is used to obtain polymer sequences comprising, at a respective residue, a mutation whereby the respective residue has an amino acid identity predicted by the trained model. Moreover, in some embodiments, the output of the neural network model is further used to score and/or rank predicted amino acid identities (e.g., specified mutations) on their ability to improve one or more protein properties of interest, such as stability or protein-protein binding affinity. For instance, in some embodiments, the model provides a functional representation for an approximation to the conditional probability of finding an amino acid identity at a single target residue site given a set of input features, with which it is possible to formulate an improved, streamlined neural network-based function (e.g., an energy function) and in turn generate improved stability and affinity scoring metrics for polymer design.

Accordingly, in some embodiments, outputted probabilities are used to swap an initial amino acid identity of a respective residue with a swap amino acid identity, where the selection of the swap amino acid identity is based on the predicted probabilities generated by the neural network. Amino acid swaps are performed by replacing an initial amino acid state (e.g., an initial amino acid identity for a respective residue) with a final amino acid state (e.g., a swapped amino acid identity for the respective residue). In some such embodiments, polymer sequences are obtained comprising, at the respective residue, a mutation whereby the initial amino acid identity of the respective residue is swapped (e.g., replaced) with a predicted amino acid identity based on the probabilities outputted by the trained model.

In some embodiments, such amino acid swapping is performed for a plurality of respective residues (e.g., target residue sites in a target design region). In some embodiments, the plurality of respective residues comprises one or more target residues in a target design region that interact with at least one other residue (e.g., residues that form binding interactions). In some such embodiments, amino acid swapping is performed for one or more of the residues in a subset of residues that participate in a binding interaction.

In some embodiments, amino acid swapping is performed for a plurality of respective target residues that have a cumulative effect on a protein function, such as a plurality of respective target residues that constitute a mutation.

For instance, in an example embodiment, a mutation is represented as a plurality of amino acid swaps for a corresponding plurality of target residues. In some such embodiments, the energy (e.g., stability) change due to a mutation (“energy function”) is a sum of contributions from each of its swaps. To evaluate this change, a “path” through each target residue site 128 in the mutation is determined, where each target site is visited once (e.g., sequentially along the path). The energy contribution from a swap at a target residue site is measured as the minus of the natural logarithm (−ln( )) of the probability of the final amino acid state over the probability of the initial amino acid state, where these probabilities 144 are obtained from the output of the neural network model 308. At each target residue site along the path, once a swap has been made at the respective target residue site, the residue feature sets 126 associated with the other target residue sites in the mutation are updated to reflect the change in the amino acid identity at the current respective target residue site.

In some embodiments, the “path” is arbitrarily selected. In other words, in some embodiments, the order in which each target residue site in the plurality of target residue sites is sequentially “visited” for the swapping and updating is randomly determined. In some such embodiments (e.g., to reduce arbitrariness and increase the accuracy of the energy function), the process of sequential selection of respective target residues and corresponding updating of residue feature sets (e.g., the path) is repeated for a plurality of iterations.

In some embodiments, the plurality of iterations is at least 10, at least 50, at least 100, at least 200, at least 300, at least 400, at least 500, at least 1000, or at least 2000 iterations. In some embodiments, the plurality of iterations is no more than 5000, no more than 2000, no more than 1000, no more than 500, or no more than 100 iterations. In some embodiments, the plurality of iterations is from 10 to 100, from 100 to 500, or from 100 to 2000. In some embodiments, the plurality of iterations falls within another range starting no lower than 10 iterations and ending no higher than 5000 iterations.

Accordingly, in some embodiments, each respective iteration of the path generates a respective polymer sequence (e.g., a polymer sequence comprising one or more swapped amino acids obtained by the sequential swapping and updating for each respective target residue in the plurality of target residues). Thus, in some embodiments, the repeating the sequential swapping and updating (e.g., the path) for the plurality of iterations generates a corresponding plurality of generated polymer sequences.

In some embodiments, a measure of central tendency is taken over the plurality of generated polymer sequences. In some embodiments, the measure of central tendency is arithmetic mean, weighted mean, midrange, midhinge, trimean, Winsorized mean, median, and/or mode.

In some embodiments, the plurality of generated polymer sequences is scored. In some embodiments, the scoring is based upon the predicted probability and/or the energy function of each respective polymer sequence in the plurality of generated polymer sequences. In some embodiments, the scoring is based upon a polypeptide property of each respective polymer sequence in the plurality of generated polymer sequences.

In some embodiments, the plurality of generated polymer sequences is ranked. In some embodiments, the ranking is based upon the predicted probability and/or the energy function of each respective polymer sequence in the plurality of generated polymer sequences. In some embodiments, the ranking is based upon a polypeptide property of each respective polymer sequence in the plurality of generated polymer sequences.

In some embodiments, the output of the neural network model is further used to automatically generate novel mutations that could simultaneously improve one or more protein properties, such as stability (e.g., protein fold), binding affinity (e.g., protein-protein binding), binding specificity (e.g., selective protein-protein binding), or a combination thereof.

Methods for scoring and ranking polymer sequences and obtaining polypeptide properties are described in further detail, for instance, in the following section entitled “Sampling methods for amino acid identity selection.”

### Sampling Methods for Amino Acid Identity Selection.

As described above, in some embodiments, outputted probabilities from the neural network are used to replace an initial amino acid identity of a respective residue with a swap amino acid identity. Systems and methods for selection of swap amino acid identities will now be described in greater detail.

Referring to Block 222, in some embodiments, the method further comprises selecting, as the identity of the respective residue, the naturally occurring amino acid having the greatest probability in the plurality of probabilities. Thus, in some embodiments, an amino acid prediction for the target residue site is made by selecting the amino acid identity for the highest-valued probability element.

In some embodiments, the method comprises selecting the amino acid identities for the top N-valued probability elements. In some embodiments, N is 1, 2, 3, 4, 5, 6, 7, 8, 9, 10, or more than 10. In some embodiments, the top N-valued probability elements for a respective residue are grouped.

In some embodiments, the selected swap amino acid identity is different from an initial amino acid identity of the respective residue. In some embodiments, the selected swap amino acid identity is such that the change in amino acid identity from the initial amino acid identity to the swap amino acid identity results in a steric complementary pair of swaps.

In some embodiments, the method includes obtaining an output for a joint probability of respective amino acid identities across several target residues in a specified target region (e.g., each respective residue in a subset of the plurality of residues for which probabilities are obtained).

In some embodiments, the obtaining an output for the joint probability uses a sampling algorithm 310. In some embodiments, the sampling algorithm is a stochastic algorithm (e.g., Gibbs sampling).

In some such embodiments, the sampling algorithm 310 samples an approximation to the joint probability by cyclically sampling the conditional probability distributions pertaining to each target residue site 128 in the specified target region (e.g., each respective residue in a subset of the plurality of residues for which probabilities 144 are obtained).

Accordingly, referring to Block 224, in some such embodiments, the method further comprises, for each respective residue in at least a subset of the plurality of residues, randomly assigning an amino acid identity to the respective residue prior to the using the plurality of atomic coordinates to obtain the corresponding residue feature set. For each respective residue in the at least a subset of the plurality of residues, a procedure is performed that comprises performing the identifying a residue and the inputting the residue feature set for the identified residue into a neural network to obtain a corresponding plurality of probabilities for the respective residue. A respective swap amino acid identity for the respective residue is obtained based on a draw from the corresponding plurality of probabilities, and when the respective swap amino acid identity of the respective residue changes the identity of the respective residue, each corresponding residue feature set in the plurality of residue feature sets affected by the change in amino acid identity is updated.

Thus, in some embodiments of the sampling algorithm, the initial amino acid identity of a respective residue 128 is randomly assigned (e.g., each respective residue 128 in the at least a subset of the plurality of residues is randomly initialized), and the performing an amino acid swap replaces the randomly assigned initial amino acid identity with a swap amino acid identity that is selected based on a draw from the outputted probabilities 144. With each sequential selection and swapping of a respective residue in the at least a subset of the plurality of residues, a corresponding updating of residue feature sets 126 affected by the swapping is performed.

In some embodiments, the subset of the plurality of residues is 10 or more, 20 or more, or 30 more residues within the plurality of residues. In some embodiments, the subset of the plurality of residues comprises between 2 and 1000 residues, between 20 and 5000 residues, more than 30 residues, more than 50 residues, or more than 100 residues. In some embodiments, the subset of the plurality of residues comprises no more than 10,000, no more than 1000, no more than 500, or no more than 100 residues. In some embodiments, the subset of the plurality of residues comprises from 100 to 1000, from 50 to 10,000, or from 10 to 500 residues. In some embodiments, the subset of the plurality of residues falls within another range starting no lower than 2 residues and ending no higher than 10,000 residues.

Referring to Block 226, in some embodiments, the procedure of Block 224 is repeated until a convergence criterion is satisfied. Referring to Block 228, in some embodiments, the convergence criterion is a requirement that the identity of none of the amino acid residues in at least the subset of the plurality of residues is changed during the last instance of the procedure performed for each residue in at least the subset of the plurality of residues. Thus, in some embodiments, after each repetition of the procedure of Block 224 (e.g., with each iteration of the sampling algorithm), the distribution of candidate swap amino acid identities shifts towards regions of the sequence space 302 that are characterized by increased stability, such that, upon convergence, the swap amino acid identities selected by the sampling algorithm (e.g., for one or more respective target residues) are deemed to be stabilizing sequences.

In some embodiments, the sampling algorithm 310 is repeated for a plurality of sampling iterations. In some such embodiments, the plurality of sampling iterations is at least 10, at least 50, at least 100, at least 200, at least 300, at least 400, at least 500, at least 1000, or at least 2000 sampling iterations. In some embodiments, the plurality of sampling iterations is no more than 5000, no more than 2000, no more than 1000, no more than 500, or no more than 100 sampling iterations. In some embodiments, the plurality of sampling iterations is from 10 to 100, from 100 to 500, or from 100 to 2000 sampling iterations. In some embodiments, the plurality of sampling iterations falls within another range starting no lower than 10 sampling iterations and ending no higher than 5000 sampling iterations.

In some embodiments, it is desirable to meet one or more benchmarks for a respective one or more polypeptide properties (e.g., design objectives for protein properties) such as enhanced protein binding affinity or specificity. In some such embodiments, the sampling algorithm 310 is expanded to include a bias (e.g., a Metropolis criterion) involving the polypeptide property to be enhanced. In some embodiments, the bias (e.g., the Metropolis criterion) imposes a constraint on the sampling in which a drawn swap is not unconditionally accepted but is treated as a conditional or “attempted” swap. If the attempted swap leads to an improvement of the respective one or more protein properties of interest, then the swap amino acid identity is accepted; however, if the attempted swap does not lead to the enhancement of the respective one or more properties, then the swap amino acid identity is accepted conditionally, based on a factor (e.g., a Boltzmann factor) which is a function of the potential change to the protein property. The biased sampling algorithm 310 can therefore be used to guide or control the evolution of a distribution of generated polymer sequences (e.g., protein designs) towards enhanced values of one or more physical properties of choice. For instance, the biased sampling algorithm 310 can be used to generate polymer sequences that improve or maintain protein stability while simultaneously enhancing one or more additional protein properties.

Accordingly, referring to Block 230, the obtaining a respective swap amino acid identity for the respective residue based on a draw from the corresponding plurality of probabilities comprises determining a respective difference, E_{final}−Eᵢₙᵢₜᵢₐₗ, between (i) a property of the polypeptide without the respective swap amino acid identity for the respective residue (Eᵢₙᵢₜᵢₐₗ) against (ii) a property of the polypeptide with the respective swap amino acid identity for the respective residue (E_{final}) to determine whether the respective swap amino acid identity for the respective residue improves the property. When the respective difference indicates that the respective swap amino acid identity for the respective residue improves the property of the polypeptide, the identity of the respective residue is changed to the respective swap amino acid identity. When the respective difference indicates that the respective swap amino acid identity for the respective residue fails to improve the property of the polypeptide, the identity of the respective residue is conditionally changed to the respective swap amino acid identity based on a function of the respective difference.

In some embodiments, the property Eᵢₙᵢₜᵢₐₗ of a respective residue comprises a plurality of protein properties. In some embodiments, the property E_{final} of a respective residue comprises a plurality of protein properties. In some such embodiments, the selection of the respective swap amino acid identity is determined on the basis of a plurality of properties for the polypeptide (e.g., protein stability, binding affinity and/or specificity). In some embodiments, the (i) property of the polypeptide without the respective swap amino acid identity for the respective residue (Eᵢₙᵢₜᵢₐₗ) is of the same type as (ii) the property of the polypeptide with the respective swap amino acid identity for the respective residue (E_{final}). Thus, in some embodiments, the difference between the property Eᵢₙᵢₜᵢₐₗ and the property E_{final} is a difference between a metric that is measured with and without the swap amino acid identity.

In some embodiments, the bias is a Metropolis condition.

Metropolis conditions are known in the art. Generally, the Metropolis algorithm depends on an assumption of detailed balance that describes equilibrium for systems whose configurations have probability proportional to the Boltzmann factor. Systems with large numbers of elements, for instance, correspond to a vast number of possible configurations. The Metropolis algorithm seeks to sample the space of possible configurations in a thermal way, by exploring possible transitions between configurations. The Metropolis condition therefore approximates a model of a thermal system, particularly for systems comprising a vast number of elements. Metropolis algorithms are further described, e.g., in Saeta, “The Metropolis Algorithm: Statistical Systems and Simulated Annealing,” available on the Internet at saeta.physics.hmc.edu/courses/p170/Metropolis.pdf, which is hereby incorporated herein by reference in its entirety.

In some embodiments, the function of the respective difference also contains an artificial temperature variable that can be adjusted to control the conditional acceptance of the attempted swap. For instance, in some implementations, attempted swaps that will lead to large declines in the protein property are less likely to be accepted than attempted swaps that will lead to small declines. However, the acceptance of larger declines can be made more likely by increasing the temperature.

Accordingly, referring to Block 232, in some embodiments, the function of the respective difference has the form e^{-(E}^{_{final}}^{-E}^{ᵢₙᵢₜᵢₐₗ}^{)/T}, wherein T is a predetermined user adjusted temperature.

The use of a user adjusted temperature to obtain better heuristic solutions to a combinatorial optimization problem has its roots in the work of Kirkpatrick et al., 1983, Science 220, 4598. Kirkpatrick et al. noted the methods used to find the low-energy state of a material, in which a single crystal of the material is first melted by raising the temperature of the material. Then, the temperature of the material is slowly lowered in the vicinity of the freezing point of the material. In this way, the true low-energy state of the material, rather than some high energy-state, such as a glass, is determined. Kirkpatrick et al. noted that the methods for finding the low-energy state of a material can be applied to other combinatorial optimization problems if a proper analogy to temperature as well as an appropriate probabilistic function, which is driven by this analogy to temperature, can be developed. The art has termed the analogy to temperature an effective temperature. It will be appreciated that any effective temperature T may be chosen. There is no requirement that the effective temperature adhere to any physical dimension such as degrees Celsius, etc. Indeed, the dimensions of the effective temperature T adopts the same units as the objective function that is the subject of the optimization. In some instances, the value for the predetermined user adjusted temperature is selected based on the amount of resources available for computation. In some instances, it has been found that the predetermined user adjusted temperature does not have to be very large to produce a substantial probability of keeping a worse score. Therefore, in some instances, the predetermined user adjusted temperature is not large.

In some embodiments, where a plurality of generated polymer sequences is obtained (e.g., by repeating the procedure of Blocks 224-230 for a plurality of iterations and/or a plurality of subsets of residues), the plurality of generated polymer sequences are scored and/or ranked. In some embodiments, the scoring and/or ranking is performed using the property of the polypeptide.

In some embodiments, the property of the polypeptide is a stability metric and/or an affinity metric. In some implementations, stability and affinity metrics are derived from a physics-based and/or knowledge-based forcefield. In some implementations, stability and affinity metrics are derived from a neural network energy function based on the probability distributions output by the model 308.

For instance, referring to Block 234, in some embodiments, the property of the polypeptide is a stability of the polypeptide in forming a heterocomplex with a polypeptide of another type. Referring to Block 236, in some embodiments, the polypeptide is an Fc chain of a first type, the polypeptide of another type is an Fc chain of a second type, and the property of the polypeptide is a stability of a heterodimerization of the Fc chain of a first type with the Fc chain of the second type. In some embodiments, the property of the polypeptide is a stability of the polypeptide in forming a homocomplex with a polypeptide of the same type. For example, in some embodiments, the polypeptide is a first Fc chain of a first type, and the property of the polypeptide is a stability of a homodimerization of the first Fc chain with a second Fc chain of the first type.

In some embodiments, referring to Block 238, the property of the polypeptide is a composite of (i) a stability of the polypeptide within a heterocomplex with a polypeptide of another type, and (ii) a stability of the polypeptide within the homocomplexes. In some embodiments, the (i) stability of the polypeptide within a heterocomplex with a polypeptide of another type includes the same type of stability measure as the (ii) stability of the polypeptide within the homocomplexes. In some embodiments, the stability of the polypeptide in the homocomplexes is defined using a weighted average of the stability of each homocomplex with the weights bound by [0,1] and sum to 1. In some embodiments, the stability of the polypeptide in the homocomplexes is a non-linear weighted average of the stability of each homocomplex with the weights bound by [0,1] and sum to 1.

In some embodiments, referring to Block 240, the property of the polypeptide is a composite of (i) a combination of a stability of the polypeptide within a heterocomplex with a polypeptide of another type and a binding specificity or binding affinity of the polypeptide for the polypeptide of another type, and (ii) a combination of a stability of the polypeptide within a homocomplex and a binding specificity or binding affinity of the polypeptide for itself to form the homocomplexes. In some embodiments, the (i) combination of the stability of the polypeptide within a heterocomplex with a polypeptide of another type and the binding specificity or binding affinity of the polypeptide for the polypeptide of another type includes the same type of stability metric and the same type of binding specificity or binding affinity metric as the (ii) combination of a stability of the polypeptide within a homocomplex and a binding specificity or binding affinity of the polypeptide for itself to form the homocomplexes. Thus, in some embodiments, the combination of stability and binding specificity or binding affinity used to characterize the polypeptide within a heterocomplex includes the same types of metrics as the combination of stability and binding specificity or binding affinity used to characterize the polypeptide within a homocomplex. In some embodiments, the stability, binding specificity or binding affinity of the polypeptide in the homocomplexes are defined using a weighted average of the stability, binding specificity or binding affinity of each homocomplex with the weights bound by [0,1] and sum to 1. In some embodiments, the stability, binding specificity or binding affinity of the polypeptide in the homocomplexes is a non-linear weighted average of the stability, binding specificity or binding affinity of each homocomplex with the weights bound by [0,1] and sum to 1.

In some embodiments, referring to Block 242, the property of the polypeptide is a stability of polypeptide, a pI of polypeptide, a percentage of positively charged residues in the polypeptide, an extinction coefficient of the polypeptide, an instability index of the polypeptide, or an aliphatic index of the polypeptide, or any combination thereof.

In some embodiments, the property of the polypeptide is selected from the group consisting of: number of amino acids (e.g., number of residues in each protein), molecular weight (e.g., molecular weight of the protein), theoretical pI (e.g., pH at which the net charge of the protein is zero (isoelectric point)), amino acid composition (e.g., percentage of each amino acid in the protein), positively charged residue 2 (e.g., percentage of positively charged residues in the protein (lysine and arginine)), positively charged residue 3 (e.g., percentage of positively charged residues in the protein (histidine, lysine, and arginine)), number of atoms (e.g., total number of atoms), carbon (e.g., total number of carbon atoms in the protein sequence), hydrogen (e.g., total number of hydrogen atoms in the protein sequence), nitrogen (e.g., total number of nitrogen atoms in the protein sequence), oxygen (e.g., total number of oxygen atoms in the protein sequence), sulphur (e.g., total number of sulphur atoms in the protein sequence), extinction coefficient all Cys (e.g., amount of light a protein absorbs at a certain wavelength assuming all Cys residues appear as half cysteines), extinction coefficient no Cys (e.g., amount of light a protein absorbs at a certain wavelength assuming no Cys residues appear as half cysteines), instability index (e.g., the stability of the protein), aliphatic index (e.g., the relative volume of the protein occupied by aliphatic side chains), GRAVY (e.g., Grand average of hydropathicity), PPR (e.g., percentage of continuous changes from positively charged residues to positively charged residues), NNR (e.g., percentage of continuous changes from negatively charged residues to negatively charged residues), PNPR (e.g., percentage of continuous changes from positively charged residues to negatively charged residues or from negatively charged residues to positively charged residues), NNRDist(x, y) (e.g., percentage of NNR from x to y (local information)), PPRDist(x, y) (e.g., percentage of PPR from x to y (local information)), PNPRDist(x, y) (e.g., percentage of PNPR from x to y (local information)), negatively charged residues (e.g., percentage of negatively charged residues in the protein), and amino acid pair ratio (e.g., percentage compositions for each of the 400 possible amino acid dipeptides)

In some embodiments, the property of the polypeptide is a physicochemical property selected from the group consisting of charged; polar; aliphatic; aromatic; small; tiny; bulky; hydrophobic; hydrophobic and aromatic; neutral, weakly and hydrophobic; hydrophilic and acidic; hydrophilic and basic; acidic; and polar and uncharged.

In some embodiments, the property of the polypeptide is a physicochemical property selected from the group consisting of steric parameter, polarizability, volume, hydrophobicity, isoelectric point, helix probability, and sheet probability.

In some embodiments, the property of the polypeptide is a protein class selected from the group consisting of transport, transcription, translation, gluconate utilization, amino acid biosynthesis, fatty acid metabolism, acetylcholine receptor inhibitor, G-protein coupled receptor, guanine nucleotide-releasing factor, fiber protein, and transmembrane.

In some embodiments, the property of the polypeptide is any suitable protein property or feature known in the art and/or disclosed herein (see, e.g., the section entitled “Residue feature sets,” above). For instance, in some embodiments, the property of the polypeptide is any of the protein properties, classes, and/or features disclosed in Lee et al., “Identification of protein functions using a machine-learning approach based on sequence-derived properties,” Proteome Science 2009, 7:27; doi:10.1186/1477-5956-7-27; and Wang et al., “Computational Protein Design with Deep Learning Neural Networks.” Nature. Sci. Rep. 8, 6349 (2018), each of which is hereby incorporated herein by reference in its entirety, and/or any substitutions, modifications, additions, deletions, or combinations thereof, as will be apparent to one skilled in the art.

Suitable embodiments for polypeptide properties further include physics-based (e.g., Amber force-field), knowledge-based, statistical, and/or structural packing-based affinity metrics (e.g., U, Electrostatic, and/or DDRW). See, for example, Cornell et al., 1995, “A Second Generation Force Field for the Simulation of Proteins,” Nucleic Acids, and Organic Molecules”, J. Am. Chem. Soc. 117: 5179-5197, and Ponder and Case, 2003, “Force fields for protein simulations,” Adv. Prot. Chem. 66, p. 27, each of which is hereby incorporated by reference herein in its entirety.

More generally, in some embodiments, the property of the polypeptide is obtained using an energy function.

In some embodiments, the property of the polypeptide is a selectivity metric and/or a specificity metric. In some such embodiments, the property of the polypeptide is obtained using the calculation Δp=pₘᵤₜ−p_{WT}, where p is the probability 144 for a respective residue, mut denotes a swap amino acid identity, and WT refers to a wild-type amino acid identity for the respective residue).

In some embodiments, the property of the polypeptide is selected from a database. Suitable databases for polypeptide properties include, but are not limited to, protein sequence databases (e.g., DisProt, InterPro, MobiDB, neXtProt, Pfam, PRINTS, PROSITE, the Protein Information Resource, SUPERFAMILY, Swiss-Prot, NCBI, etc.), protein structure databases (e.g., the Protein Data Bank (PDB), the Structural Classification of Proteins (SCOP), the Protein Structure Classification (CATH) Database, Orientations of Proteins in Membranes (OPM) database, etc.), protein model databases (e.g., ModBase, Similarity Matrix of Proteins (SIMAP), Swiss-model, AAindex, etc.), protein-protein and molecular interaction databases (e.g., BioGRID, RNA-binding protein database, Database of Interacting Proteins, IntAct, etc.), protein expression databases (e.g., Human Protein Atlas, etc.), and/or physicochemical databases (e.g., the Amino acid Physico-chemical properties Database (APD)). See, for example, Mathura and Kolippakkam, “APDbase: Amino acid Physico-chemical properties Database,” Bioinformation 1(1): 2-4 (2005), which is hereby incorporated herein by reference in its entirety. In some embodiments, the database is SKEMPI. See, for example, Jankauskaité et al., “SKEMPI 2.0: an updated benchmark of changes in protein-protein binding energy, kinetics and thermodynamics upon mutation.” Bioinformatics 35(3), 462-469 (2019), which is hereby incorporated herein by reference in its entirety.

In some embodiments, a respective swap amino acid identity is obtained for all or a subset of the plurality of residues using any of the methods disclosed herein, thereby obtaining a plurality of generated polymer sequences.

In some such embodiments, the method comprises obtaining generated polymer sequences including, at each respective residue 128 in the all or a subset of the plurality of residues, a mutation whereby the initial amino acid identity of the respective residue is swapped (e.g., replaced) with a swap amino acid identity selected based on the probabilities 144 outputted by the trained model 308 (e.g., the generated polymer sequence comprises one or more mutated residues).

In some embodiments, a respective swap amino acid identity is obtained for a respective one or more residues in a respective one or more chemical species (e.g., proteins and/or protein complexes) using any of the methods disclosed herein, thereby obtaining a plurality of generated polymer sequences.

Accordingly, in some embodiments, the method comprises obtaining generated polymer sequences including, for each respective chemical species in a plurality of chemical species, at each respective residue in the corresponding one or more residues for the respective chemical species, a mutation whereby the initial amino acid identity of the respective residue is swapped (e.g., replaced) with a swap amino acid identity selected based on the probabilities 144 outputted by the trained model 308 (e.g., each generated polymer sequence in a plurality of generated polymer sequences comprises one or more mutated residues).

In some embodiments, the plurality of chemical species comprises at least 2, at least 3, at least 4, at least 5, at least 10, at least 15, at least 20, or at least 50 chemical species. In some embodiments, the plurality of chemical species comprises no more than 100, no more than 50, no more than 20, or no more than 10 chemical species. In some embodiments, the plurality of chemical species is from 2 to 10, from 5 to 20, or from 10 to 50 chemical species. In some embodiments, the plurality of chemical species falls within another range starting no lower than 2 chemical species and ending no higher than 100 chemical species.

In some embodiments, the obtaining a respective swap amino acid identity for a respective one or more residues in a respective one or more chemical species occurs simultaneously.

For instance, in some embodiments, enhanced binding affinity or specificity design simulations can be used to track several chemical species, such as ligands and receptors in bound and unbound states. As a result, in a respective sampling algorithm, when a swap is accepted, for each respective chemical species in a plurality of chemical species affected by the swap, a data array representation (e.g., one or more residue feature sets) for the respective chemical species is updated accordingly. As an illustrative example, enhancement of HetFc binding specificity comprises simultaneous tracking of 7 chemical species including three bound (one HetFc and two HomoFc) and four unbound (single Fc) chemical species, such that a swap being applied to the heterodimer will also result in this swap occurring at two residue sites on one of the two homodimers and at one residue site on each of two unbound Fc chains.

### Neural Network Architecture.

Generally, in some embodiments, a neural network comprises of a plurality of inputs (e.g., residue feature set 312 and/or a data frame), a corresponding first hidden layer comprising a corresponding plurality of hidden neurons, where each hidden neuron in the corresponding plurality of hidden neurons (i) is fully connected to each input in the plurality of inputs, (ii) is associated with a first activation function type, and (iii) is associated with a corresponding parameter (e.g., weight) in a plurality of parameters 138 for the neural network, and one or more corresponding neural network outputs, where each respective neural network output in the corresponding one or more neural network outputs (i) directly or indirectly receives, as input, an output of each hidden neuron in the corresponding plurality of hidden neurons, and (ii) is associated with a second activation function type.

In some embodiments, the neural network comprises a plurality of hidden layers. As described above, hidden layers are located between input and output layers (e.g., to capture additional complexity). In some embodiments, where there is a plurality of hidden layers, each hidden layer may have a same respective number of neurons. In some embodiments, each hidden neuron (e.g., in a respective hidden layer in a neural network) is associated with an activation function that performs a function on the input data (e.g., a linear or non-linear function). Generally, the purpose of the activation function is to introduce nonlinearity into the data such that the neural network is trained on representations of the original data and can subsequently “fit” or generate additional representations of new (e.g., previously unseen) data. Selection of activation functions (e.g., a first and/or a second activation function) is dependent on the use case of the neural network, as certain activation functions can lead to saturation at the extreme ends of a dataset (e.g., tanh and/or sigmoid functions). For instance, in some embodiments, an activation function (e.g., a first and/or a second activation function) is selected from any of the activation functions disclosed herein and described in greater detail below. In some embodiments, each hidden neuron is further associated with a parameter (e.g., a weight and/or a bias value) that contributes to the output of the neural network, determined based on the activation function. In some embodiments, the hidden neuron is initialized with arbitrary parameters (e.g., randomized weights). In some alternative embodiments, the hidden neuron is initialized with a predetermined set of parameters.

FIGS. 3-D illustrate an exemplary architecture for a neural network model 308, in accordance with some embodiments of the present disclosure. The model 308 includes two stages in which, upon input of a residue feature set 312, a first stage 314 comprising a one-dimensional convolution neural sub-network architecture (CNN) is followed by a second stage 316 comprising a fully-connected sub-network architecture (FCN). The first stage 1D-CNN sub-network consists of two parallel (e.g., “left” branch and “right” branch), sequential series of convolution layers. As illustrated in FIG. 3D, there are four levels 320 in this sub-network, each level consisting of two parallel convolution layers.

Accordingly, referring to Block 244, in some embodiments, the neural network 308 comprises a first-stage one-dimensional sub-network architecture 314 that feeds into a fully connected neural network 316 having a final node that outputs the probability of each respective naturally occurring amino acid as a twenty element probability vector 318 in which the twenty elements sum to 1.

Referring again to FIG. 3D, each respective branch of the first-stage convolutional neural network is convolved with a respective filter. For instance, in some embodiments, the convolution comprises “windowing” a filter of a specified size (e.g., 1×1, 2×2, 1×N_{f}, 2×N_{f}, etc.) across the plurality of elements in an input data frame. As the filter moves along (e.g., in accordance with a specified stride), each window is convolved according to a specified function (e.g., an activation function, average pooling, max pooling, batch normalization, etc.). At the end of each respective level, after the activation stage of a convolution for that level, the outputs from the two parallel, coincident convolution layers (e.g., the left and right branch layers) are concatenated and passed to each of the two parallel, coincident convolution layers of the next level.

Thus, referring to Block 246, in some embodiments, the first-stage one-dimensional sub-network architecture comprises a plurality of pairs of convolutional layers (e.g., a plurality of levels), including a first pair of convolutional layers and a second pair of convolutional layers (e.g., Level 1 and Level 2). The first pair of convolutional layers (e.g., Level 1) includes a first component convolutional layer (e.g., 320-1-1) and a second component convolutional layer (e.g., 320-1-2) that each receive the residue feature set 312 during the inputting. The second pair of convolutional layers (e.g., Level 2) includes a third component convolutional layer (e.g., 320-2-1) and a fourth component convolutional layer (e.g., 320-2-2). The first component convolutional layer (e.g., 320-1-1) of the first pair of convolutional layers and the third component convolutional layer (e.g., 320-2-1) of the second pair of convolutional layers each convolve with a first filter dimension (e.g., 140-1). The second component convolutional layer (e.g., 320-1-2) of the first pair of convolutional layers and the fourth component convolutional layer (e.g., 320-2-2) of the second pair of convolutional layers each convolve with a second filter dimension (e.g., 140-2) that is different than the first filter dimension. A concatenated output of the first and second component convolutional layers of the first pair of convolutional layers (e.g., 320-1-1 and 320-1-2) serves as input to both the third component (e.g., 320-2-1) and fourth component (e.g., 320-2-2) convolutional layers of the second pair of convolutional layers.

While Block 246 illustrates an interaction of inputs and outputs passed between a first layer and a second layer of an example first-stage convolutional neural network, in some embodiments, the number of pairs of convolutional layers (e.g., levels) is any number of pairs of convolutional layers (e.g., levels). In some such embodiments, the passing of outputs from the first level as inputs to the second level is performed for each subsequent pair of levels in the first-stage convolutional neural network.

For instance, referring to Block 248, in some embodiments, the plurality of pairs of convolutional layers comprises between two and ten pairs of convolutional layers. Each respective pair of convolutional layers includes a component convolutional layer that convolves with the first filter dimension, each respective pair of convolutional layers includes a component convolutional layer that convolves with the second filter dimension, and each respective pair of convolutional layers other than a final pair of convolutional layers in the plurality of pairs of convolutional layers passes a concatenated output of the component convolutional layers of the respective convolutional layer into each component convolutional layer of another pair of convolutional layers in the plurality of convolutional layers.

In some embodiments, a respective convolution layer is characterized by the number of filters 140 in the respective layer, as well as the shape (e.g., height and width) of each of these filters. In some embodiments, the first-stage convolutional neural network comprises two filter types (e.g., 140-1 and 140-2), each characterized by a distinct “height.”

Accordingly, referring to Block 250, in some embodiments, the neural network is characterized by a first convolution filter and a second convolutional filter that are different in size. Referring to Block 252, in some embodiments, the first filter dimension is one and the second filter dimension is two. In some embodiments, the first filter dimension is two and the second filter dimension is one. In some embodiments, a respective filter dimension is 1, 2, 3, 4, 5, 6, 7, 8, 9, 10, or more than 10. In some embodiments, a respective filter dimension is between 1 and 10, between 1 and 5, or between 1 and 3. In some embodiments, a respective filter dimension is equal to K, where K is the number of neighboring residues for a respective residue in an input data frame 312-A. In some embodiments, a respective filter dimension is equal to N_{f}, where N_{f} is the number of features, in a residue feature set, for a respective residue relative to a respective neighboring residue in K neighboring residues in an input data frame 312-A. In some embodiments, a respective filter dimension is any value between 1 and K or between 1 and N_{f} In some embodiments, a respective filter dimension refers to the height and/or the width of a respective input data frame 312-A

In some embodiments, a respective filter is characterized by a respective stride (e.g., a number of elements by which a filter moves across an input tensor or data frame). In some embodiments, the stride is at least 1, at least 2, at least 3, at least 4, at least 5, at least 6, at least 7, at least 8, at least 9, or at least 10. In some embodiments, the stride is no more than 50, no more than 30, no more than 20, no more than 10, no more than 9, no more than 8, no more than 7, no more than 6, no more than 5, no more than 4, no more than 3, no more than 2, or no more than 1. In some embodiments, the stride is from 1 to 10, from 2 to 30, or from 5 to 50. In some embodiments, the stride falls within another range starting no lower than 1 and ending no higher than 50.

In some embodiments, the stride is size one, and the filters, when striding, progress down the height axis of the residue feature data frame.

In some embodiments, the first-stage convolutional neural network 314 further comprises a final pooling layer that performs a pooling operation to the concatenated data frame outputted by the final pair of convolutional layers (e.g., the final level) of the first-stage CNN. In some embodiments, the pooling layer is used to reduce the number of elements and/or parameters associated with the data frame used as input for the second-stage fully-connected network (e.g., to reduce the computational complexity). Accordingly, in some embodiments, the pooling operation collapses the outputted data frame along one or both axes. In some embodiments, the pooling operation collapses the outputted data frame along its height axis. In some embodiments, the pooling operation is average pooling, max pooling, global average pooling, and/or 1-dimensional global average pooling.

In some embodiments, the pooled output data frame is inputted into the second stage 316 of the model. In some embodiments, the second stage is a fully-connected traditional neural network. In the example embodiment, the final output of the model 308 consists of a single neuron (e.g., node) that outputs a probability vector 318 including a respective probability 144 for each of a plurality of amino acid identities possible at a target residue site.

In some embodiments, the model comprises one or more normalization layers. For example, in some embodiments, the model comprises one or more batch normalization layers in the first-stage CNN and/or the second-stage FCN. In some embodiments, a respective normalization layer performs a normalization step, including, but not limited to batch normalization, local response normalization and/or local contrast normalization. In some embodiments, a respective normalization layer encourages variety in the response of several function computations to the same input. In some embodiments, the one or more normalization layers are applied prior to a respective activation stage for a respective convolutional layer in the model. In some embodiments, the model comprises, for each respective convolutional layer in the model (e.g., in the first-stage CNN and/or in the second-stage FCN), a respective batch normalization layer that is applied prior to the respective activation stage for the respective convolutional layer.

In some embodiments, the model comprises one or more activation layers that perform a corresponding one or more activation functions. For example, in some embodiments, an activation layer is a layer of neurons that applies the non-saturating activation function f(x)=max(0, x). More generally, in some embodiments, a respective neuron in a plurality of neurons in the model applies an activation function to generate an output. In some embodiments, a respective activation function in the one or more activation layers increases the nonlinear properties of the decision function and of the overall model without affecting the receptive fields of a convolutional layer. In other embodiments, an activation layer includes other functions to increase nonlinearity, for example, the saturating hyperbolic tangent function f(x)=tanh, f(x)=|tanh(x)|, and the sigmoid function f(x)=(1+e−x)−1. Nonlimiting examples of other activation functions found in some embodiments for the model include, but are not limited to, logistic (or sigmoid), softmax, Gaussian, Boltzmann-weighted averaging, absolute value, linear, rectified linear, bounded rectified linear, soft rectified linear, parameterized rectified linear, average, max, min, some vector norm LP (for p=1, 2, 3, . . . , ∞), sign, square, square root, multiquadric, inverse quadratic, inverse multiquadric, polyharmonic spline, and thin plate spline.

In some embodiments, the model 308, the first-stage CNN 314, and/or the second-stage FCN 316 comprises a plurality of parameters (e.g., weights and/or hyperparameters).

In some embodiments, the plurality of parameters comprises at least 10, at least 50, at least 100, at least 500, at least 1000, at least 2000, at least 5000, at least 10,000, at least 20,000, at least 50,000, at least 100,000, at least 200,000, at least 500,000, at least 1 million, at least 2 million, at least 3 million, at least 4 million or at least 5 million parameters. In some embodiments, the plurality of parameters comprises no more than 8 million, no more than 5 million, no more than 4 million, no more than 1 million, no more than 500,000, no more than 100,000, no more than 50,000, no more than 10,000, no more than 5000, no more than 1000, or no more than 500 parameters. In some embodiments, the plurality of parameters comprises from 10 to 5000, from 500 to 10,000, from 10,000 to 500,000, from 20,000 to 1 million, or from 1 million to 5 million parameters. In some embodiments, the plurality of parameters falls within another range starting no lower than 10 parameters and ending no higher than 8 million parameters.

In some embodiments, the plurality of parameters for a respective convolutional layer in the model 308, the first-stage CNN 314, and/or the second-stage FCN 316 comprises at least 100, at least 500, at least 1000, at least 2000, at least 5000, at least 10,000, at least 20,000, at least 50,000, at least 100,000, at least 200,000, at least 500,000, at least 1 million, at least 2 million, or at least 3 million parameters. In some embodiments, the plurality of parameters for a respective convolutional layer in the model 308, the first-stage CNN 314, and/or the second-stage FCN 316 comprises no more than 5 million, no more than 4 million, no more than 1 million, no more than 500,000, no more than 100,000, no more than 50,000, no more than 10,000, no more than 5000, or no more than 1000 parameters. In some embodiments, the plurality of parameters for a respective convolutional layer in the model 308, the first-stage CNN 314, and/or the second-stage FCN 316 comprises from 100 to 1000, from 1000 to 10,000, from 2000 and 200,000, from 8000 and 1 million, or from 30,000 and 3 million parameters. In some embodiments, the plurality of parameters for a respective convolutional layer in the model 308, the first-stage CNN 314, and/or the second-stage FCN 316 falls within another range starting no lower than 100 parameters and ending no higher than 5 million parameters.

In some embodiments, a parameter comprises a number of hidden neurons. In some embodiments, the plurality of hidden neurons in the model 308, the first-stage CNN 314, and/or the second-stage FCN 316 (e.g., across one or more hidden layers) is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 neurons. In some embodiments, the plurality of hidden neurons in the model 308, the first-stage CNN 314, and/or the second-stage FCN 316 is at least 100, at least 500, at least 800, at least 1000, at least 2000, at least 3000, at least 4000, at least 5000, at least 6000, at least 7000, at least 8000, at least 9000, at least 10,000, at least 15,000, at least 20,000, or at least 30,000 neurons. In some embodiments, the plurality of hidden neurons in the model 308, the first-stage CNN 314, and/or the second-stage FCN 316 is no more than 30,000, no more than 20,000, no more than 15,000, no more than 10,000, no more than 9000, no more than 8000, no more than 7000, no more than 6000, no more than 5000, no more than 4000, no more than 3000, no more than 2000, no more than 1000, no more than 900, no more than 800, no more than 700, no more than 600, no more than 500, no more than 400, no more than 300, no more than 200, no more than 100, or no more than 50 neurons. In some embodiments, the plurality of hidden neurons in the model 308, the first-stage CNN 314, and/or the second-stage FCN 316 is from 2 to 20, from 2 to 200, from 2 to 1000, from 10 to 50, from 10 to 200, from 20 to 500, from 100 to 800, from 50 to 1000, from 500 to 2000, from 1000 to 5000, from 5000 to 10,000, from 10,000 to 15,000, from 15,000 to 20,000, or from 20,000 to 30,000 neurons. In some embodiments, the plurality of hidden neurons in the model 308, the first-stage CNN 314, and/or the second-stage FCN 316 falls within another range starting no lower than 2 neurons and ending no higher than 30,000 neurons.

In some embodiments, a parameter comprises a number of convolutional layers (e.g., hidden layers) in the first-stage CNN. In some embodiments, the CNN comprises at least 2, at least 3, at least 4, at least 5, at least 6, at least 7, at least 8, at least 9, at least 10, at least 20, at least 30, at least 40, at least 50, or at least 100 convolutional layers. In some embodiments, the CNN comprises no more than 200, no more than 100, no more than 50, or no more than 10 convolutional layers. In some embodiments, the CNN comprises from 1 to 10, from 1 to 20, from 2 to 80, or from 10 to 100 convolutional layers. In some embodiments, the CNN comprises a plurality of convolutional layers that falls within another range starting no lower than 1 layer and ending no higher than 100 layers.

In some embodiments, a parameter comprises an activation function. For example, in some embodiments, one or more hidden layers in the model 308, the first-stage CNN 314, and/or the second-stage FCN 316 is associated with one or more activation functions. In some embodiments, an activation function in the one or more activation functions is tanh, sigmoid, softmax, Gaussian, Boltzmann-weighted averaging, absolute value, linear, rectified linear unit (ReLU), bounded rectified linear, soft rectified linear, parameterized rectified linear, average, max, min, sign, square, square root, multiquadric, inverse quadratic, inverse multiquadric, polyharmonic spline, swish, mish, Gaussian error linear unit (GeLU), or thin plate spline. In some embodiments, an activation function in the one or more activation functions is any of the operation and/or functions disclosed herein. Other suitable activation functions are possible, as will be apparent to one skilled in the art.

In some embodiments, a parameter comprises a number of training epochs (e.g., for training the model 308, the first-stage CNN 314, and/or the second-stage FCN 316). In some embodiments, the number of training epochs is at least 5, at least 10, at least 20, at least 50, at least 100, at least 200, at least 300, at least 500, at least 1000, or at least 2000 epochs. In some embodiments, the number of training epochs is no more than 5000, no more than 1000, no more than 500, no more than 300, or no more than 100 epochs. In some embodiments, the number of epochs is from 20 to 100, from 100 to 1000, from 50 to 800, or from 200 to 1000 epochs. In some embodiments, the number of training epochs falls within another range starting no lower than 10 epochs and ending no higher than 5000 epochs.

In some embodiments, a parameter comprises a learning rate. In some embodiments, the learning rate is at least 0.0001, at least 0.0005, at least 0.001, at least 0.005, at least 0.01, at least 0.05, at least 0.1, at least 0.2, at least 0.3, at least 0.4, at least 0.5, at least 0.6, at least 0.7, at least 0.8, at least 0.9, or at least 1. In some embodiments, the learning rate is no more than 1, no more than 0.9, no more than 0.8, no more than 0.7, no more than 0.6, no more than 0.5, no more than 0.4, no more than 0.3, no more than 0.2, no more than 0.1 no more than 0.05, no more than 0.01, or less. In some embodiments, the learning rate is from 0.0001 to 0.01, from 0.001 to 0.5, from 0.001 to 0.01, from 0.005 to 0.8, or from 0.005 to 1. In some embodiments, the learning rate falls within another range starting no lower than 0.0001 and ending no higher than 1. In some embodiments, the learning rate further comprises a learning rate decay (e.g., a reduction in the learning rate over one or more epochs). For example, a learning decay rate could be a reduction in the learning rate of 0.5 over 5 epochs or a reduction of 0.1 over 20 epochs). In some embodiments, the learning rate is a differential learning rate.

In some embodiments, a parameter includes a regularization strength (e.g., L2 weight penalty, dropout rate, etc.). For instance, in some embodiments, the model 308, the first-stage CNN 314, and/or the second-stage FCN 316 is trained using a regularization on a corresponding parameter (e.g., weight) of each hidden neuron in the plurality of hidden neurons. In some embodiments, the regularization includes an L1 or L2 penalty. In some embodiments, the dropout rate is at least 1%, at least 2%, at least 5%, at least 10%, at least 15%, at least 20%, at least 30%, at least 40%, or at least 50%. In some embodiments, the dropout rate is no more than 80%, no more than 50%, no more than 20%, no more than 15%, or no more than 10%. In some embodiments, the dropout rate is from 1% to 90%, from 5% to 50%, from 10% to 40%, or from 15% to 30%.

In some embodiments, a parameter comprises an optimizer.

In some embodiments, a parameter comprises a loss function. In some embodiments, the loss function is mean square error, flattened mean square error, quadratic loss, mean absolute error, mean bias error, hinge, multi-class support vector machine, and/or cross-entropy. In some embodiments, the loss function is a gradient descent algorithm and/or a minimization function.

In some embodiments, a respective convolutional layer and/or pair of convolutional layers (e.g., level) has the same or different values for a respective parameter as another respective convolutional layer and/or pair of convolutional layers.

In some embodiments, a respective parameter is a hyperparameter (e.g., a tunable value). In some embodiments, the hyperparameter value is tuned (e.g., adjusted) during training. In some embodiments, the hyperparameter value is determined based on the specific elements of a training dataset and/or one or more input data frames (e.g., residue feature sets). In some embodiments, the hyperparameter value is determined using experimental optimization. In some embodiments, the hyperparameter value is determined using a hyperparameter sweep. In some embodiments, the hyperparameter value is assigned based on prior template or default values.

In some embodiments, the model 308, the first-stage CNN 314, and/or the second-stage FCN 316 comprises any model architecture disclosed herein (see, Definitions: Model), or any substitutions, modifications, additions, deletions, or combinations thereof, as will be apparent to one skilled in the art. In some embodiments, the model 308 is an ensemble model comprising at least a first model and a second model, where each respective model in the ensemble model comprises any of the embodiments for model architectures disclosed herein.

In some embodiments, the model 308, the first-stage CNN 314, and/or the second-stage FCN 316 comprises a multilayer neural network, a deep convolutional neural network, a fully connected neural network, a visual geometry convolutional neural network, a residual neural network, a residual convolutional neural network, an SPR, and/or a combination thereof.

Other suitable model architectures contemplated for use in the present disclosure are further disclosed in Wang et al., “Computational Protein Design with Deep Learning Neural Networks.” Nature. Sci. Rep. 8, 6349 (2018), which is hereby incorporated herein by reference in its entirety.

### Training Neural Networks.

Generally, training a model (e.g., a neural network) comprises updating the plurality of parameters for the respective model through backpropagation (e.g., gradient descent). First, a forward propagation is performed, in which input data (e.g., a training dataset comprising one or more residue feature sets) is accepted into the model, and an output is calculated based on the selected activation function and an initial set of parameters. In some embodiments, parameters are randomly assigned (e.g., initialized) for the untrained or partially trained model. In some embodiments, parameters are transferred from a previously saved plurality of parameters or from a pre-trained model (e.g., by transfer learning).

A backward pass is then performed by calculating an error gradient for each respective parameter corresponding to each respective unit in each layer of the model, where the error for each parameter is determined by calculating a loss (e.g., error) based on the model output (e.g., the predicted value) and the input data (e.g., the expected value or true labels; here, amino acid identities). Parameters (e.g., weights) are then updated by adjusting the value based on the calculated loss, thereby training the model.

For example, in some general embodiments of machine learning, backpropagation is a method of training a model with hidden layers comprising a plurality of weights (e.g., embeddings). The output of an untrained model (e.g., the predicted probabilities for amino acid identities for a respective residue) is first generated using a set of arbitrarily selected initial weights. The output is then compared with the original input (e.g., the actual amino acid identity of the respective residue) by evaluating an error function to compute an error (e.g., using a loss function). The weights are then updated such that the error is minimized (e.g., according to the loss function). In some embodiments, any one of a variety of backpropagation algorithms and/or methods are used to update the plurality of weights, as will be apparent to one skilled in the art.

In some embodiments, the loss function is any of the loss functions disclosed herein (see, e.g., the section entitled “Neural network architecture,” above). In some embodiments, training the untrained or partially trained model comprises computing an error in accordance with a gradient descent algorithm and/or a minimization function. In some embodiments, the error function is used to update one or more parameters (e.g., weights) in a model by adjusting the value of the one or more parameters by an amount proportional to the calculated loss, thereby training the model. In some embodiments, the amount by which the parameters are adjusted is metered by a learning rate hyperparameter that dictates the degree or severity to which parameters are updated (e.g., smaller or larger adjustments). Thus, in some embodiments, the training updates all or a subset of the plurality of parameters (e.g., 500 or more parameters) based on a learning rate.

In some embodiments, the training further uses a regularization on the corresponding parameter of each hidden neuron in the corresponding plurality of hidden neurons in the model. For example, in some embodiments, a regularization is performed by adding a penalty to the loss function, where the penalty is proportional to the values of the parameters in the trained or untrained model. Generally, regularization reduces the complexity of the model by adding a penalty to one or more parameters to decrease the importance of the respective hidden neurons associated with those parameters. Such practice can result in a more generalized model and reduce overfitting of the data. In some embodiments, the regularization includes an L1 or L2 penalty. For example, in some preferred embodiments, the regularization includes an L2 penalty on lower and upper parameters. In some embodiments, the regularization comprises spatial regularization or dropout regularization. In some embodiments, the regularization comprises penalties that are independently optimized.

Accordingly, referring to Block 254, in some embodiments, the method includes instructions for training the neural network to minimize a cross-entropy loss function across a training dataset of reference protein residue sites labeled by their amino acid designations obtained from a dataset of protein structures. In some embodiments, this loss function measures the total cost of errors made by the model in making the amino acid label predictions across a PDB-curated dataset. Thus, parameters 138 in the model are learned by training on a training dataset including PDB structure-sequence data.

In some embodiments, the training dataset comprises a plurality of training examples, where each respective training example comprises a residue feature set for a respective residue in a plurality of polypeptide residues and an amino acid identity (e.g., label) of the respective residue. In some embodiments, the plurality of training examples includes at least 1000, at least 10,000, at least 100,000, at least 200,000, at least 500,000, at least 1 million, at least 1.5 million, at least 2 million, or at least 5 million training examples. In some embodiments, the plurality of training examples includes no more than 10 million, no more than 5 million, no more than 1 million, or no more than 100,000 training examples. In some embodiments, the plurality of training examples includes from 10,000 to 500,000, from 100,000 to 1 million, from 200,000 to 2 million, or from 1 million to 10 million training examples. In some embodiments, the plurality of training examples falls within another range starting no lower than 1000 training examples and ending no higher than 10 million training examples.

In some embodiments, the training dataset comprises, for each respective residue feature set in the plurality of residue feature sets, backbone-dependent (BB) features for the respective residue.

In some embodiments, the training dataset comprises, for each respective residue feature set in the plurality of residue feature sets, neighboring amino acid (side chain (SC)) features for the respective residue.

In some embodiments, the model is an ensemble model comprising at least a first model and a second model, where the first model is trained on BB features only, and the second model is trained on both BB features and SC features. For instance, in some embodiments, the first model is used for initial amino acid identity prediction (e.g., sequence assignment) and the second model is used for amino acid identity refinement (e.g., sequence refinement).

In some embodiments, the model is trained over a plurality of epochs (e.g., including any number of epochs as disclosed herein; see, for instance, the section entitled “Neural network architecture,” above).

In some embodiments, training the model forms a trained model following a first evaluation of an error function. In some such embodiments, training the model forms a trained model following a first updating of one or more parameters based on a first evaluation of an error function. In some alternative embodiments, training the model comprises at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million evaluations of an error function. In some such embodiments, training the model comprises at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million updatings of one or more parameters based on the at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million evaluations of an error function.

In some embodiments, a trained model is formed when the model satisfies a minimum performance requirement. For example, in some embodiments, a trained model is formed when the error calculated for the trained model, following an evaluation of an error function across one or more training datasets, satisfies an error threshold. In some embodiments, the error calculated by the error function across one or more training datasets satisfies an error threshold when the error is less than 20 percent, less than 18 percent, less than 15 percent, less than 10 percent, less than 5 percent, or less than 3 percent. Thus, for example, in some embodiments, a trained model is formed when the best performance is achieved.

In some embodiments, the model performance is measured using a training loss metric, a validation loss metric, and/or a mean absolute error.

For instance, in some embodiments, the model performance is measured by validating the model using one or more residue feature sets in a validation dataset. In some such embodiments, a trained model is formed when the model satisfies a minimum performance requirement based on a validation training. Thus, in some embodiments, training accuracy and/or loss is optimized using a training dataset, and performance of the model is validated on the validation dataset.

In some embodiments, any suitable method for validation can be used, including but not limited to K-fold cross-validation, advanced cross-validation, random cross-validation, grouped cross-validation (e.g., K-fold grouped cross-validation), bootstrap bias corrected cross-validation, random search, and/or Bayesian hyperparameter optimization.

In some embodiments, the validation dataset comprises a plurality of validation examples, where each respective validation example comprises a residue feature set for a respective residue in a plurality of polypeptide residues and an amino acid identity (e.g., label) of the respective residue. In some embodiments, the plurality of validation examples includes at least 100, at least 1000, at least 10,000, at least 100,000, at least 200,000, at least 500,000, or at least 1 million validation examples. In some embodiments, the plurality of validation examples includes no more than 2 million, no more than 1 million, or no more than 100,000 validation examples. In some embodiments, the plurality of validation examples includes from 10,000 to 500,000, from 100,000 to 1 million, from 150,000 to 500,000, or from 200,000 to 800,000 million validation examples. In some embodiments, the plurality of validation examples falls within another range starting no lower than 1000 validation examples and ending no higher than 2 million validation examples. In some embodiments, the validation dataset does not contain any residue feature sets in common with the training dataset.

### Additional Embodiments

In some embodiments, as described above, a plurality of swap amino acid identities for a corresponding one or more residues and/or a corresponding one or more chemical species (e.g., a plurality of generated polymer sequences) is obtained as outputs from the neural network. For instance, in some such embodiments, the method comprises obtaining a plurality of generated polymer sequences, each comprising one or more mutated residues, where, for each respective mutated residue, the initial amino acid identity of the respective residue is swapped (e.g., replaced) with a swap amino acid identity selected based on the probabilities outputted by the trained model. In some embodiments, as described above, the plurality of generated polymer sequences are provided as a ranked list (e.g., based on a probability, a score, and/or a protein property).

In some embodiments, one or more polymer sequences in the plurality of generated polymer sequences is a novel polymer sequence (e.g., not included in a polypeptide training dataset or polypeptide database).

In some embodiments, the method further comprises clustering the plurality of generated polymer sequences comprising the amino acid identities predicted by the neural network model.

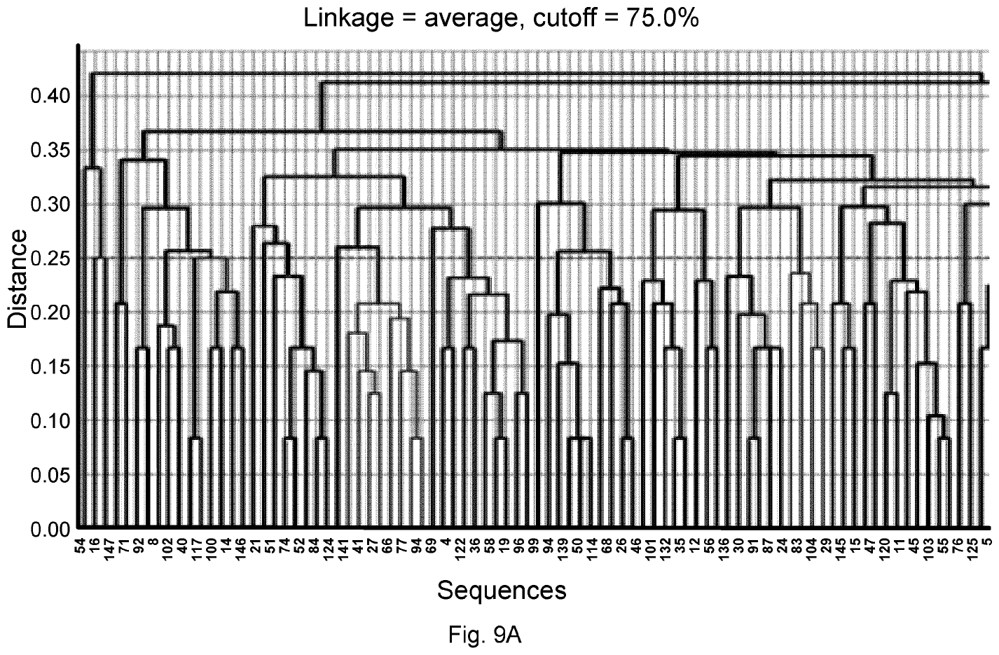

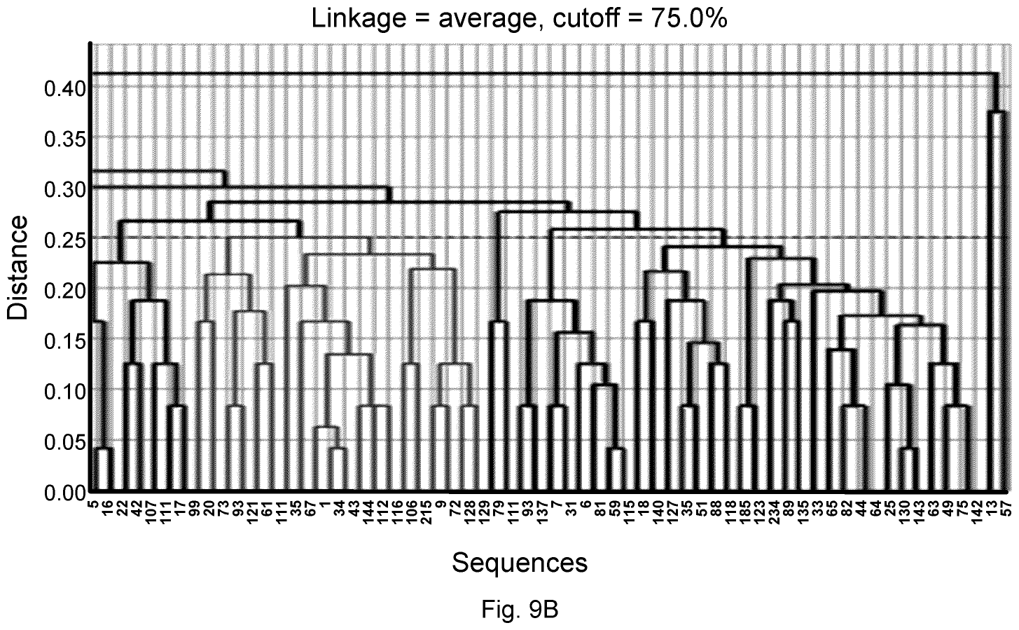

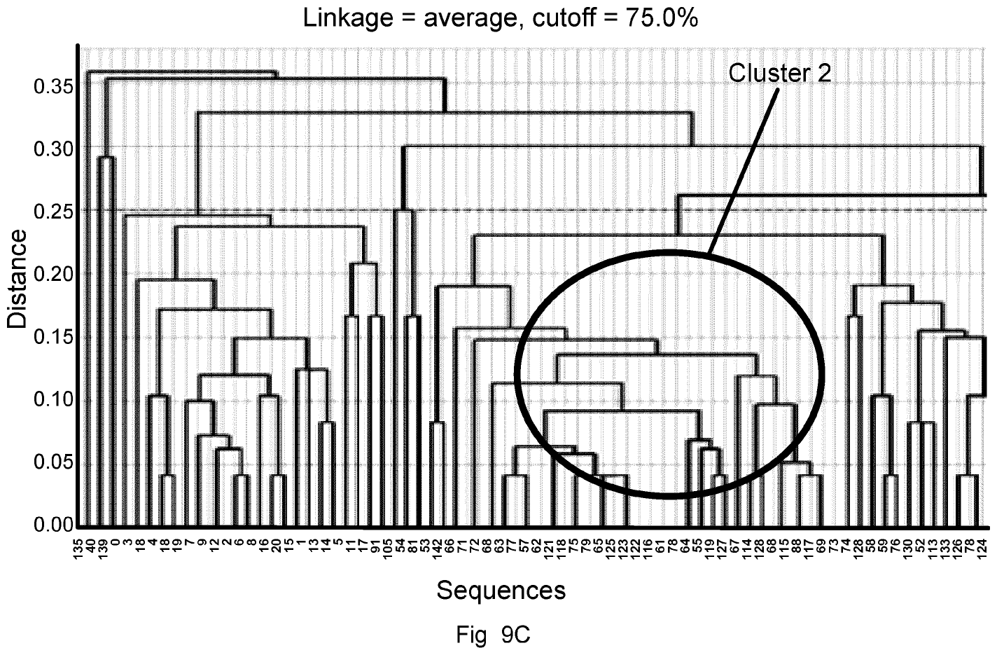

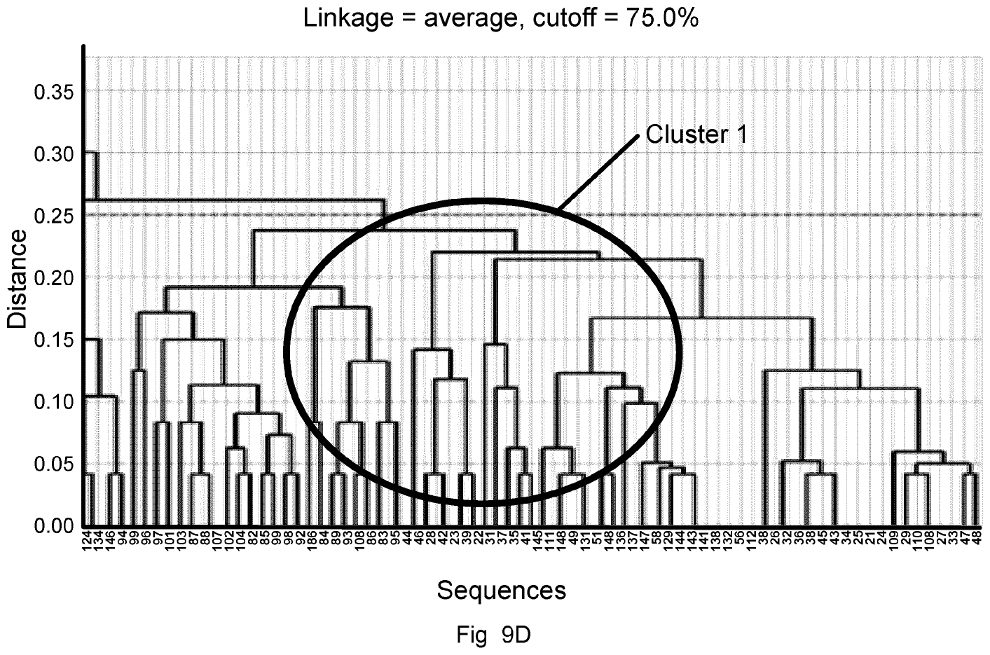

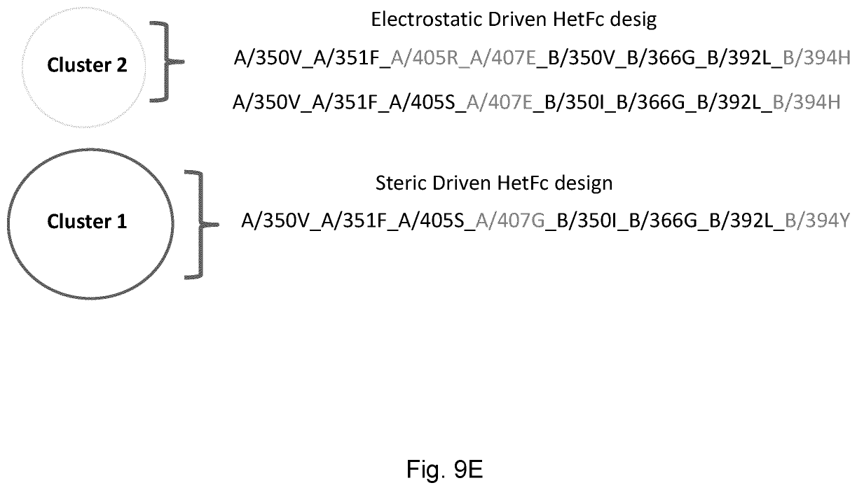

In some embodiments, the clustering reduces the plurality of generated polymer sequences into groups of meaningfully distinct structural conformations. For instance, consider the case in which there are two generated polymer sequences (e.g., mutated polymer structures) that only differ by half a degree in a single terminal dihedral angle. Such sequences are not deemed to be meaningfully distinct and therefore fall into the same cluster in some instances of the present disclosure. Advantageously, the example provides for reducing the plurality of generated polymer sequences into a reduced set of sequences without losing information about meaningfully distinct conformations found in the plurality of generated polymer sequences. This is done in some use cases by clustering on side chains individually and/or the backbone individually (e.g., on a residue by residue basis). This is done in other use cases by (i) clustering on side chains individually and (ii) separately clustering based on a structural metric associated with the main chain of each contiguous block of main chains in the plurality of sequences, thereby deriving a set of main chain clusters for each contiguous block of main chain coordinates. Regardless of which use case is performed, if there is a meaningful shift in any side chain or any backbone between two of the generated polymer sequences, even if the two sequences are otherwise structurally very similar, the clustering ultimately will not group the two conformations into the same cluster and thus obscure that difference. Meaningfully distinct clusters of sequences (e.g., electrostatic-driven clustering and steric-driven clustering) are illustrated below in Example 3, with reference to FIGS. 9A-C.

Clustering is described in further detail above (see, Definitions: Models). Particular exemplary clustering techniques that can be used include, but are not limited to, hierarchical clustering (agglomerative clustering using nearest-neighbor algorithm, farthest-neighbor algorithm, the average linkage algorithm, the centroid algorithm, or the sum-of-squares algorithm), maximal linkage agglomerative clustering, complete linkage hierarchical clustering, k-means clustering, fuzzy k-means clustering algorithm, Jarvis-Patrick clustering, and steepest-descent clustering. In some instances, the clustering is a residue by residue clustering. In some embodiments, the clustering imposes a root-mean-square distance (RMSD) cutoff on the coordinates of the side chain atoms or the main chain atoms of each respective polymer sequence.

Referring to Block 256, in some embodiments, the at least one program of the presently disclosed computer system further comprises instructions for using the probability for each respective naturally occurring amino acid for the respective residue to determine an identity of the respective residue, using the respective residue to update an atomic structure of the polypeptide, and using the updated atomic structure of the polypeptide to determine, in silico, an interaction score between the polypeptide and a composition.

In some embodiments, the polypeptide is modified using, for the respective residue, the naturally occurring amino acid having the greatest probability in the plurality of probabilities, thereby forming a modified polypeptide, in order to improve a stability of the polypeptide.

In some embodiments, the polypeptide is modified using, for the respective residue, the naturally occurring amino acid having the greatest probability in the plurality of probabilities, thereby forming a modified polypeptide, in order to improve an affinity of the polypeptide for another protein.

In some embodiments the polypeptide is modified using, for the respective residue, the naturally occurring amino acid having the greatest probability in the plurality of probabilities, thereby forming a modified polypeptide, in order to improve a selectivity of the polypeptide in binding a second protein relative to the polypeptide binding a third protein.

In some embodiments the modified polypeptide is used as a treatment of a medical condition associated with the polypeptide. In some such embodiments the treatment is a composition comprising the modified polypeptide and one or more excipient and/or one or more pharmaceutically acceptable carrier and/or one or more diluent. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

An exemplary carrier is pharmaceutically “acceptable” in the sense of being compatible with the other ingredients of the composition (e.g., the composition comprising the modified polymer) and not injurious to the patient. The compositions may conveniently be presented in unit dosage form and may be prepared by any methods well known in the art of pharmacy. Such methods include the step of bringing into association the active ingredient with the carrier that constitutes one or more accessory ingredients. In general, the compositions are prepared by uniformly and intimately bringing into association the active ingredient with liquid carriers or finely divided solid carriers or both, and then if necessary shaping the product.

Exemplary compounds, compositions or combinations of the present disclosure (e.g., the composition comprising the modified polymer) formulated for intravenous, intramuscular or intraperitoneal administration, and a compound of the invention or a pharmaceutically acceptable salt, solvate or prodrug thereof may be administered by injection or infusion.

Injectables for such use can be prepared in conventional forms, either as a liquid solution or suspension or in a solid form suitable for preparation as a solution or suspension in a liquid prior to injection, or as an emulsion. Carriers can include, for example, water, saline (e.g., normal saline (NS), phosphate-buffered saline (PBS), balanced saline solution (BSS)), sodium lactate Ringer's solution, dextrose, glycerol, ethanol, and the like; and if desired, minor amounts of auxiliary substances, such as wetting or emulsifying agents, buffers, and the like can be added. Proper fluidity can be maintained, for example, by using a coating such as lecithin, by maintaining the required particle size in the case of dispersion and by using surfactants.

The compound, composition or combinations of the present disclosure (e.g., the composition comprising the modified polymer) may also be suitable for oral administration and may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste. In another embodiment, the compound of formula (I) or a pharmaceutically acceptable salt, solvate or prodrug is orally administerable.

A tablet may be made by compression or molding, optionally with one or more accessory ingredients. Compressed tablets may be prepared by compressing in a suitable machine the active ingredient (e.g., the composition comprising the modified polymer) in a free-flowing form such as a powder or granules, optionally mixed with a binder (e.g inert diluent, preservative disintegrant (e.g. sodium starch glycolate, cross-linked polyvinyl pyrrolidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

The compound, composition or combinations of the present disclosure (e.g., the composition comprising the modified polymer) may be suitable for topical administration in the mouth including lozenges comprising the active ingredient in a flavored base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

The compound, composition or combinations of the present disclosure (e.g., the composition comprising the modified polymer) may be suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the compounds of the invention.

The compound, composition or combination of the present disclosure (e.g., the composition comprising the modified polymer) may be suitable for parenteral administration include aqueous and non-aqueous isotonic sterile injection solutions which may contain anti-oxidants, buffers, bactericides and solutes which render the compound, composition or combination isotonic with the blood of the intended recipient; and aqueous and non-aqueous sterile suspensions which may include suspending agents and thickening agents. The compound, composition or combination may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the active ingredients particularly mentioned above, the composition or combination of this present disclosure (e.g., the composition comprising the modified polymer) may include other agents conventional in the art having regard to the type of composition or combination in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

In some embodiments the medical condition is inflammation or pain. In some embodiments the medical condition is a disease. For example, in some embodiments the medical condition is asthma, an autoimmune disease, autoimmune lymphoproliferative syndrome (ALPS), cholera, a viral infection, Dengue fever, an E. coli infection, Eczema, hepatitis, Leprosy, Lyme Disease, Malaria, Monkeypox, Pertussis, a Yersinia pestis infection, primary immune deficiency disease, prion disease, a respiratory syncytial virus infection, Schistosomiasis, gonorrhea, genital herpes, a human papillomavirus infection, chlamydia, syphilis, Shigellosis, Smallpox, STAT3 dominant-negative disease, tuberculosis, a West Nile viral infection, or a Zika viral infection. In some embodiments, the medical condition is a disease references in Lippincott, Williams & Wilkins, 2009, Professional Guide to Diseases, 9^{th} Edition, Wolters Kluwere, Philadelphia, Pennsylvania, which is hereby incorporated by reference.

In some embodiments, the method further comprises treating the medical condition by administering the treatment to a subject in need of treatment of the medical condition.

Referring to Block 258, in some such embodiments, the polypeptide is an enzyme, the composition is being screened in silico to assess an ability to inhibit an activity of the enzyme, and the interaction score is a calculated binding coefficient of the composition to the enzyme. Referring to Block 260, in other such embodiments, the protein is a first protein, the composition is a second protein being screened in silico to assess an ability to bind to the first protein in order to inhibit or enhance an activity of the first protein, and the interaction score is a calculated binding coefficient of the second protein to the first protein. Referring to Block 262, in still other such embodiments, the protein is a first Fc fragment of a first type, the composition is a second Fc fragment of a second type, and the interaction score is a calculated binding coefficient of the second Fc fragment to the first Fc fragment.

Another aspect of the present disclosure provides a non-transitory computer readable storage medium storing one or more computational modules for polymer sequence prediction, the one or more computational modules collectively comprising instructions for obtaining a plurality of atomic coordinates for at least the main chain atoms of a polypeptide, where the polypeptide comprises a plurality of residues. The instructions further comprise using the plurality of atomic coordinates to encode each respective residue in the plurality of residues into a corresponding residue feature set in a plurality of residue feature sets.

The corresponding residue feature set comprises, for the respective residue and for each respective residue having a C_{α} carbon that is within a nearest neighbor cutoff of the C_{α} carbon of the respective residue, (i) an indication of the secondary structure of the respective residue encoded as one of helix, sheet and loop, (ii) a relative solvent accessible surface area of the C_{α}, N, C, and O backbone atoms of the respective residue, and (iii) cosine and sine values of backbone dihedrals ϕ, ψ and ω of the respective residue. The corresponding residue feature set further comprises a C_{α} to C_{α} distance of each neighboring residue having a C_{α} carbon within a threshold distance of the C_{α} carbon of the respective residue, and an orientation and position of a backbone of each neighboring residue relative to the backbone residue segment of the respective residue in a reference frame centered on C_{α} atom of the respective residue.

The instructions further include identifying a respective residue in the plurality of residues and inputting the residue feature set corresponding to the identified respective residue into a neural network (e.g, comprising at least 500 parameters), thereby obtaining a plurality of probabilities, including a probability for each respective naturally occurring amino acid.

Another aspect of the present disclosure provides a method for polymer sequence prediction, the method comprising, at a computer system comprising a memory, obtaining a plurality of atomic coordinates for at least the main chain atoms of a polypeptide, where the polypeptide comprises a plurality of residues. The method includes using the plurality of atomic coordinates to encode each respective residue in the plurality of residues into a corresponding residue feature set in a plurality of residue feature sets.

The corresponding residue feature set comprises, for the respective residue and for each respective residue having a C_{α} carbon that is within a nearest neighbor cutoff of the G carbon of the respective residue, (i) an indication of the secondary structure of the respective residue encoded as one of helix, sheet and loop, (ii) a relative solvent accessible surface area of the C_{α}, N, C, and O backbone atoms of the respective residue, and (iii) cosine and sine values of backbone dihedrals ϕ, ψ and ω of the respective residue. The corresponding residue feature set further includes a C_{α} to C_{α} distance of each neighboring residue having a C_{α} carbon within a threshold distance of the C_{α} carbon of the respective residue, and an orientation and position of a backbone of each neighboring residue relative to the backbone residue segment of the respective residue in a reference frame centered on C_{α} atom of the respective residue.

The method further includes identifying a respective residue in the plurality of residues and inputting the residue feature set corresponding to the identified respective residue into a neural network (e.g, comprising at least 500 parameters), thereby obtaining a plurality of probabilities, including a probability for each respective naturally occurring amino acid.

Yet another aspect of the present disclosure provides a computer system having one or more processors, and memory storing one or more programs for execution by the one or more processors, the one or more programs comprising instructions for performing any of the methods and/or embodiments disclosed herein. In some embodiments, any of the presently disclosed methods and/or embodiments are performed at a computer system having one or more processors, and memory storing one or more programs for execution by the one or more processors.

Still another aspect of the present disclosure provides a non-transitory computer readable storage medium storing one or more programs configured for execution by a computer, the one or more programs comprising instructions for carrying out any of the methods disclosed herein.

### EXAMPLES

### Example 1—Performance Measures of Automated Sequence Design (ZymeSwapNet) Using Backbone-Dependent Features

A deep neural network (DNN) 308 in accordance with an embodiment of the present disclosure (ZymeSwapNet) was trained to predict the amino acid identity (label) at an arbitrary target residue site on a protein. The DNN comprised two sub-networks, including a 1-dimensional convolution network (1D-CNN) 314 feeding into a fully-coupled network (FCN) 316. Input features (X₀) 312 to the DNN assigned to each respective identified residue 128 in a plurality of residues for a respective polypeptide 122 (e.g., a target residue site 128) included backbone-dependent (BB) features. For instance, BB features included, for each target residue 128 and each respective neighboring residue 132 in K˜20 neighboring residues, the corresponding backbone angles, the surface area, and the secondary structure; and, for each target residue 128, the distances and directions to the K neighboring backbone (C_{α}) atoms about each target site and the relative orientations of target and K neighboring backbone segments. Input features for the target residue were formulated into a feature matrix of dimensions K×N_{f}, where K is the number of neighboring residues and N_{f} is the number of residue features (e.g., 130, 132, 135) for the respective target residue.

The DNN comprising approximately 500,000 internal parameters was trained on 1,800,000 training examples (e.g., residues) to learn the hierarchy of higher-order features; in other words, the DNN was trained to recognize patterns in feature space X₀ that prove advantageous for predicting the label of an amino acid identity. Training was performed by minimizing a loss function over the training subset, for a number of epochs (e.g., iterations). Each epoch represents one cycle through the plurality of training examples in the training subset. The trained DNN was tested on 300,000 test examples (e.g., residues), and the output (e.g., a vector 318 of 20 amino acid probabilities for the target residue 128) was plotted for assessment of instantaneous accuracy over the testing subset.

FIGS. 4A-C illustrate the prediction accuracy of ZymeSwapNet over the test subset. For instance, FIGS. 4A-B illustrate a prediction accuracy of approximately 46 to 49 percent for the testing subset (indicated as “NN model” in FIG. 4A and “val” in FIG. 4B). Compared to other state-of-the-art neural network models, ZymeSwapNet's accuracy and performance is comparable, despite the simplicity of its architecture. Compared with Graph Networks trained on atomic coordinates as input features, ZymeSwapNet is trained on a small set of local pre-engineered input features and therefore does not require a complex architecture to achieve the comparable level of prediction accuracy. For instance, as illustrated in FIG. 4A, compared to traditional computational protein design methods that utilize physical force-fields acting on all-atom protein models, ZymeSwapNet's accuracy (“NN model”) was ˜10 to 15 percent higher than the traditional method (“Literature”) in wild-type sequence recovery tests when fed only backbone-dependent features. Benchmark prediction accuracies for the traditional method (“Literature”) were obtained using an SPR network architecture, retrained using the plurality of residue features as inputs. See, for example, Wang et al., “Computational Protein Design with Deep Learning Neural Networks.” Nature. Sci. Rep. 8, 6349 (2018), which is hereby incorporated herein by reference in its entirety.

Furthermore, when the input X₀ is expanded to include neighboring residue amino acid identities (“side chain features” (SC), described further in Example 2, below) about a target residue site in addition to the backbone-dependent features for the target residue, ZymeSwapNet's accuracy over the testing subset was boosted to 54 to 56 percent, as illustrated in FIG. 4C (“val”).

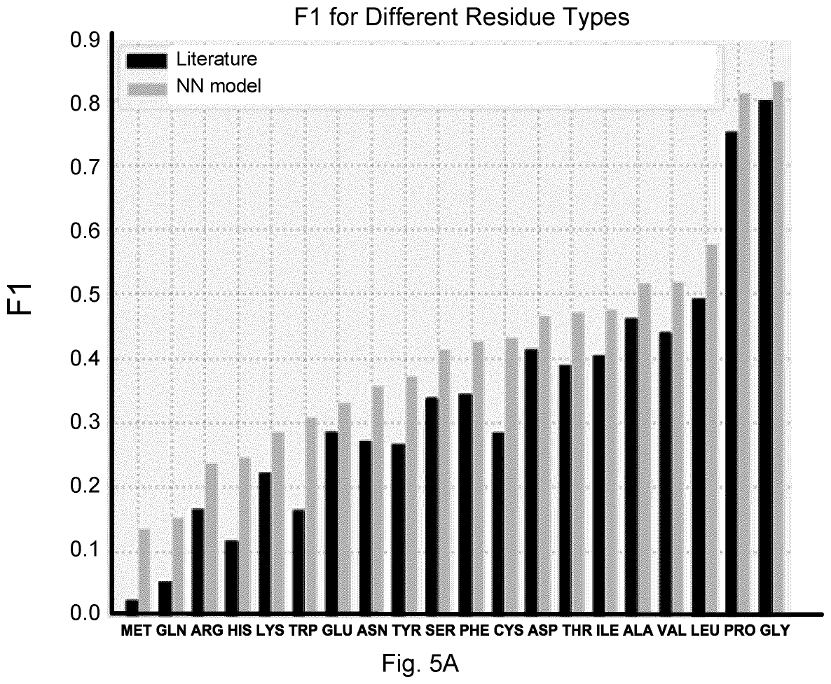

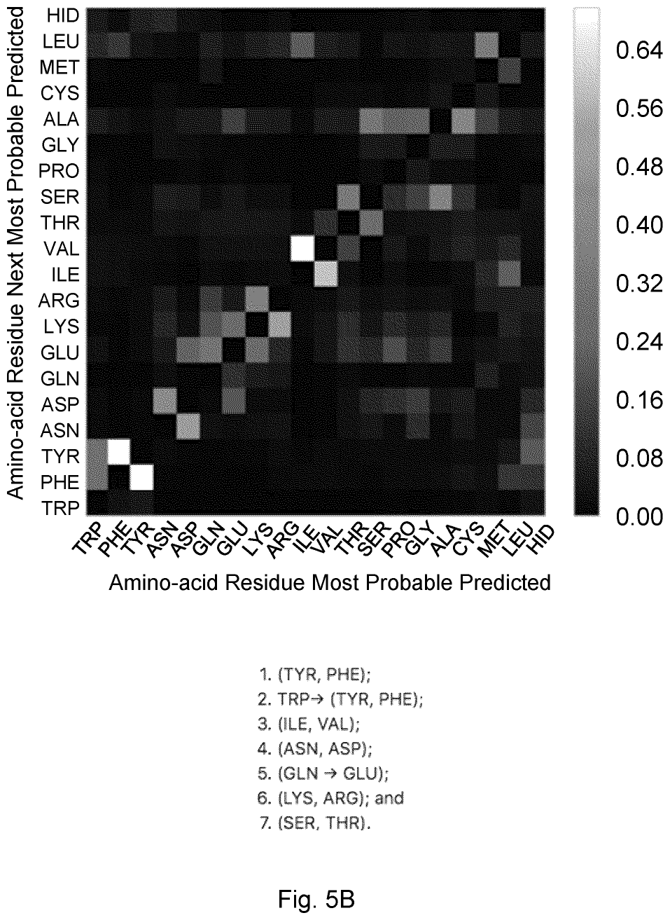

Accuracy of amino acid identity predictions are further illustrated in FIG. 5A, in which F1 scores are presented for each amino acid identity (“RES”). Here, F1 scores indicate the accuracy of predictions, where higher scores indicate higher accuracy and a score of 1 indicates perfect prediction. The graph provides F1 scores for each of ZymeSwapNet (“NN model”) and the traditional method obtained using the SPR network architecture described above (“Literature”). Notably, ZymeSwapNet outperformed the traditional method for all amino acid identities. FIG. 5B provides a heatmap illustrating physical groupings of amino acid types identified by the ZymeSwapNet model, where the most probable predicted amino acids are grouped with the next most probable predicted amino acids (e.g., for each respective amino acid identity in the plurality of amino acid identities, when the respective amino acid has the maximum-valued predicted probability, the respective amino acid is grouped with the one or more amino acids having the next-highest-valued predicted probability).

### Example 2—Fc Prediction of Automated Sequence Design (ZymeSwapNet) Using Backbone-Dependent Features

In some implementations, a neural network model (e.g., ZymeSwapNet) is trained to effectively predict a sequence (e.g., an amino acid identity) given a protein structure or fold (e.g., by applying ZymeSwapNet repeatedly across every residue site along the protein backbone) and is further used to discover potentially stabilizing mutations.

For example, referring to FIGS. 6A-C, an IgG1 Fc domain structure was selected for comparison of ZymeSwapNet amino acid predictions against experimental results. The IgG1 Fc domain structure was previously re-engineered by protein engineers (PE), using manual design, to become a heterodimer (HetFc). For instance, FIG. 6B illustrates a schematic for designing Fc variants for a respective antibody, including a heterodimeric Fc (“HetFc,” left box) and a homodimeric Fc (“HomoFc,” right box). For this Example, design objectives for the HetFc included improved stability (stability enhancement) and heterodimeric specificity (selectivity driver), but in some implementations can also include binding affinity. Manual design of the IgG1 Fc domain structure resulted in first-stage negative design rounds in which Fc stability was lost after applying a critical steric complementarity pair (ScP) of swaps. These swaps favored the specific binding of the HetFc domain over the two associated homodimeric Fc domains. See, for instance, Von Kreudenstein et al., “Improving biophysical properties of a bispecific antibody scaffold to aid developability.” mAbs 5, 5, 646-654 (2013), which is hereby incorporated herein by reference in its entirety.

Referring to FIG. 6A, the table shows the 8 amino acid swaps from the “best” reported HetFc design (see, e.g., Von Kreudenstein et al., above). “Swap Site” indicates the location of the amino acid swap in the Fc domain sequence, while “Manual Design Swap” indicates the amino acid identity used for the manually designed amino acid swap. The table further provides the four most probable amino acids predicted by the neural network model (ZymeSwapNet), from left (more probable) to right (less probable). In FIG. 6A, ZymeSwapNet was trained using backbone-dependent (BB) features only, as described above in Example 1. Predictions aligned with manual designs are highlighted in bold text, predictions aligned with the wild-type identity are underlined, residues that contribute to selective binding (heterodimeric specificity) of HetFc are indicated by asterisks, and residues that contribute to increased stability are indicated by arrows. Notably, ZymeSwapNet accurately reproduced the stabilizing swaps chosen by the protein engineers. In other words, ZymeSwapNet correctly recommended swaps that were either the same or very similar to stabilizing swaps that were identified by protein engineers in order to build back stability that was lost during the earlier negative design rounds. Interestingly, these stabilizing swaps complementing the ScP swaps were predicted by ZymeSwapNet to be more probable than the corresponding wild-type amino-acid identities at the same residue site locations. Thus, the output of ZymeSwapNet can be used to identify sites where a swap has large probability of increasing protein stability.

ZymeSwapNet also accurately predicted the wild-type amino-acid type at the ScP locations, at A/407 and B/394. For instance, the swap site A_Y407V (i.e., on chain A, sequence position 407) was used by protein engineers to swap in a “small residue,” but ZymeSwapNet trained on only BB features accurately predicted the wild-type A/407.TYR. Similarly, the swap site B_T394W (i.e., on chain B, sequence position 394) was used by protein engineers to swap in a “bulky residue,” but ZymeSwapNet trained on only BB features accurately predicted the wild-type B/394.THR. To improve the predictions generated by ZymeSwapNet, neighboring residue amino acid identities and corresponding neighboring residue features (e.g., “side chain features” (SC)) were included as input features to add more contextual information.

Side chain features included the addition of K=19 neighboring residue amino acid identities as input features, including encodings of 7 physicochemical features (e.g., steric parameter; polarizability; volume; hydrophobicity; isoelectric point; helix probability; and sheet probability). In particular, the include of side chain features is beneficial in that ZymeSwapNet is better able to capture the response of the polypeptide to sequence changes. For example, if a small residue is initially replaced by a large residue, then the probability of the presence of a neighboring large residue is likely to decrease while the probability of the presence of a neighboring small residue is likely to increase. This improvement in prediction accuracy is described in Example 1, above, with reference to FIG. 4C, where the addition of neighboring residue features increases the accuracy of target site amino acid prediction to 54-56% across the test set of ˜300,000 target residue sites. FIG. 6C further illustrates the effect of using neighboring residue features as inputs on the prediction probabilities for amino acid identities obtained by ZymeSwapNet. First, using the IgG1 Fc domain, a “bulky residue” was swapped at a swap site (e.g., B/T394W) and a corresponding “small residue” was identified (e.g., at A/407) across the binding interface. Probabilities were generated from ZymeSwapNet, which was trained given backbone (BB) and side chain (SC) features. In the legend, entropy values for swap identities at A/407 are shown prior to and after the application of a B/T394W swap. Entropy values (“S”) of 0 and 1 correspond to respectively a flat and sharply peaked amino-acid probability distribution at a target residue site, in the current case at position A/407. Moreover, prediction probabilities for each respective amino acid identity vary depending on the presence or absence of the “bulky residue” (e.g., the B/T394W swap). These data therefore indicate that ZymeSwapNet can respond to local changes in amino-acid identity.

The findings obtained from automated sequence design suggested that the probability distributions output by ZymeSwapNet could be used to formulate neural network based stability and affinity metrics to score and rank mutations. Accordingly, ZymeSwapNet derived affinity metrics were tested on a set of mutations listed in the well-known, open-source protein-protein affinity database, SKEMPI. See, for example, Jankauskaité et al., “SKEMPI 2.0: an updated benchmark of changes in protein-protein binding energy, kinetics and thermodynamics upon mutation.” Bioinformatics 35(3), 462-469 (2019), which is hereby incorporated herein by reference in its entirety. As illustrated in FIG. 7A, affinity predictions made based on the ZymeSwapNet metrics correlated well with experimental protein binding affinity measurements (Pearson r˜0.5, Kendall Tau ˜0.3), such that the degree of this agreement was comparable to that exhibited by other standard physics-based (e.g., Amber force-field) and knowledge-based and/or statistical affinity metrics (e.g., structural packing-based affinity metrics such as LJ, Electrostatic, and/or DDRW).

Unlike the calculation of the latter metrics, the ZymeSwapNet metrics calculations are extremely fast because they do not require a lengthy side-chain conformational repacking; furthermore, as a result, ZymeSwapNet metric calculations avoid being beleaguered by issues associated with repacking: the choice of force-field; the treatment of protonation state; the potential deficiencies arising from the reliance on a rotamer library. This makes the calculation of ZymeSwapNet stability and affinity metrics ideal for fast inspections on the impacts of set of mutations applied to a starting protein structure.

For instance, probabilities distributions can be used to determine scores (e.g., changes in probability), which can in turn be used to identify amino acid “hot-spots” for selecting swap amino acid identities for polymer sequence generation. In some such embodiments, the probability of an amino acid identity can be used as a measure of the stability of the polypeptide. FIG. 7B provides a heatmap illustrating the change in probability for a given residue type from the wild-type sequence. Change in probability is defined as the probability of the mutated amino acid (e.g., the swap amino acid identity) minus the probability of the wild-type amino acid, and this difference was rescaled by a factor of 100 for better readability. Positive changes in probability indicate hot-spots (e.g., as depicted by deeply shaded elements outlined by the boxes).

In another example, ZymeSwapNet output probabilities can be used to determine a specificity metric. For instance, as illustrated in FIG. 7C, for the IgG1 Fc domain, the specificity metric is calculated as a measure of the difference in the binding affinity of the HetFc from these affinities for the two corresponding HomoFc. Thus, a more negative value indicates greater preferential binding of HetFc over the two HomoFc. FIG. 7C illustrates the specificity metric for swap amino acid identities obtained from ZymeSwapNet trained on BB features as well as neighboring side-chain amino-acid identities (SC features) to a target residue site. The inclusion of SC features in the set of ZymeSwapNet input features (“SC) boosts the predictive performance of these metrics and allows the modeling of coupled effects between swap sites. In particular, FIG. 7C illustrates that the ability to train on coupled effects increases the preferential binding of HetFc over the HomoFc, as evidenced by the decreased specificity metric values over all amino acid identities when swap-coupling is enabled. This boosted effect is more clearly observed in the presence of local changes in amino acid identity, for instance, by turning on or off the critical Steric Complementarity Pair (ScP) design of swaps that drive the selective formation of heterodimeric Fc domain (e.g., specificity). Thus, preferential binding of HetFc is highest when ScP design swaps are present and when swap-coupling is enabled for amino acid predictions.

### Example 3—Biased Gibbs DNN Sampling in Automated Sequence Design (ZymeSwapNet)

To handle also fulfilling additional, chosen physical objectives like specificity, a method was performed to bias the acceptance of the swaps drawn from the ZymeSwapNet probability distributions. Biasing drives the distribution of sequences generated towards enhanced values of the chosen physical property of interest.

As outlined, ZymeSwapNet can quickly rank mutations, but it can also be used in the context of a sampling algorithm within a computational automated sequence design workflow, which rapidly proposes novel mutations or designs. Here these workflows can be constructed so that the output mutations or designs potentially satisfy multiple protein design objectives. In some embodiments, the sampling workflow can be performing using, as input, a starting design structure and a list of target regions to be sampled over in order to generate favorable target region sequences. There is no restriction to the selection of these residues; in other words, the residues in the target region do not have to be contiguous in protein sequence position.

FIGS. 8A-C illustrate the design of a HetFc where the two objectives are the enhancements to stability of the HetFc and the binding specificity to this heterodimer over the two competing homodimers. The sampling workflow was performed on a selection of target regions spanning the Fc interface. See, e.g., Von Kreudenstein et al., “Improving biophysical properties of a bispecific antibody scaffold to aid developability.” mAbs 5, 5, 646-654 (2013), which is hereby incorporated herein by reference in its entirety.

As the specificity bias is transitioned from “OFF” to “ON,” the ensemble of sampled sequences shifts towards smaller value of specificity for a plurality of metrics. For instance, smaller values of specificity (e.g., greater preferential binding of HetFc over the HomoFc) was observed for the neural network derived (DNN) specificity metrics (FIG. 8A), but also for physically-derived (dAmber: FIG. 8B; and dDDRW: FIG. 8C) specificity metrics. In these samplings, the change in the binding specificity function given a swap, which is entered into the Metropolis condition, was calculated from DNN-based energy function giving a DNN measure of the changes in stability due to the swap for the bound and unbound chemical species involved. It was also observed that as the Metropolis specificity bias was increased, the distribution of generated HetFc designs shifted towards specificity metric values that heavily favored HetFc over HomoFc binding. This effect was observed for all such metrics, whether derived from ZymeSwapNet-based, physics-based, or knowledge-based energies.

It was further observed that the stochastic sampling algorithm, which incorporates using the conditional probability represented by ZymeSwapNet to make instantaneous amino-acid assignments at target residue sites, was capable of generating novel HetFc designs, as well as the Steric Complementarity Pair (ScP) designs very similar to those discovered by protein engineers through multiple rounds of rational design and experimental verification (see, Example 2, above). Given the specific set of HetFc designs examined across the rounds in this study, as the HetFc purity of a design increased, the distribution of specificity metric values, derived from our Swap-Net based energy, shifted towards values representing a more favorable binding of HetFc over HomoFc, thereby demonstrating another correlation with experiment.

Additional information was derived from clustering analysis of generated polymer sequences. For example, as illustrated in FIGS. 9A-E, for an example target region on the IgG1 Fc domain, a dendrogram produced by hierarchical clustering of generated sequences with specificity bias turned “ON” (FIGS. 9C and 9D) produced large clusters that were not present when the specificity bias was turned “OFF” (FIGS. 9A and 9B). FIG. 9E illustrates representative sequences from each of the two dominant dendrogram clusters identified in FIGS. 9C and 9D. The generated design ensemble clusters into groups guided by different physical principles, e.g., electrostatics and steric complementarity. Accordingly, the presently disclosed framework can be used to significantly reduce human effort to quickly generate an ensemble of potential candidates that meet design objectives and that can be further characterized using downstream experiments and applications.

### CONCLUSION

The methods illustrated in FIGS. 2A, 2B, 2C, 2D, 2E, 2F, and 2G may be governed by instructions that are stored in a computer readable storage medium and that are executed by at least one processor of at least one server. Each of the operations shown in FIGS. 2A, 2B, 2C, 2D, 2E, 2F, and 2G may correspond to instructions stored in a non-transitory computer memory or computer readable storage medium. In various implementations, the non-transitory computer readable storage medium includes a magnetic or optical disk storage device, solid state storage devices such as Flash memory, or other non-volatile memory device or devices. The computer readable instructions stored on the non-transitory computer readable storage medium may be in source code, assembly language code, object code, or other instruction format that is interpreted and/or executable by one or more processors.

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the exemplary configurations may be implemented as a combined structure or component. Similarly, structures and functionality presented as a single component may be implemented as separate components. These and other variations, modifications, additions, and improvements fall within the scope of the implementation(s).

It will also be understood that, although the terms “first,” “second,” etc. may be used herein to describe various elements, these elements should not be limited by these terms. These terms are only used to distinguish one element from another. For example, a first contact could be termed a second contact, and, similarly, a second contact could be termed a first contact, which changing the meaning of the description, so long as all occurrences of the “first contact” are renamed consistently and all occurrences of the second contact are renamed consistently. The first contact and the second contact are both contacts, but they are not the same contact.

The terminology used herein is for the purpose of describing particular implementations only and is not intended to be limiting of the claims. As used in the description of the implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined (that a stated condition precedent is true)” or “if (a stated condition precedent is true)” or “when (a stated condition precedent is true)” may be construed to mean “upon determining” or “in response to determining” or “in accordance with a determination” or “upon detecting” or “in response to detecting” that the stated condition precedent is true, depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. A computer system for polymer sequence prediction, the computer system comprising:
one or more processors; and
memory addressable by the one or more processors, the memory storing at least one program for execution by the one or more processors, wherein the at least one program comprises instructions for:
(A) obtaining a plurality of atomic coordinates for at least the main chain atoms of a polypeptide, wherein the polypeptide comprises a plurality of residues;
(B) using the plurality of atomic coordinates to encode each respective residue in the plurality of residues into a corresponding residue feature set in a plurality of residue feature sets, wherein the corresponding residue feature set comprises:
for the respective residue and for each respective residue having a C_{α} carbon that is within a nearest neighbor cutoff of the C carbon of the respective residue (i) an indication of the secondary structure of the respective residue encoded as one of helix, sheet and loop, (ii) a relative solvent accessible surface area of the C_{α}, N, C, and O backbone atoms of the respective residue, and (iii) cosine and sine values of the backbone dihedrals ϕ, ψ and ω of the respective residue,
a C_{α} to C_{α} distance of each neighboring residue having a C_{α} carbon within a threshold distance of the C_{α} carbon of the respective residue, and
an orientation and position of a backbone of each neighboring residue relative to the backbone residue segment of the respective residue in a reference frame centered on C atom of the respective residue;
(C) identifying a respective residue in the plurality of residues; and
(D) inputting the residue feature set corresponding to the identified respective residue into a neural network comprising at least 500 parameters thereby obtaining a plurality of probabilities, including a probability for each respective naturally occurring amino acid.

2. The computer system of claim 1, wherein the at least one program further comprises instructions for selecting, as the identity of the respective residue, the naturally occurring amino acid having the greatest probability in the plurality of probabilities.

3. The computer system of claim 1, wherein the at least one program further comprises:
for each respective residue in at least a subset of the plurality of residues, randomly assigning an amino acid identity to the respective residue prior to the using (B),
for each respective residue in the at least a subset of the plurality of residues, performing a procedure that comprises:
performing the identifying (C) and the inputting (D) to obtain a corresponding plurality of probabilities for the respective residue,
obtaining a respective swap amino acid identity for the respective residue based on a draw from the corresponding plurality of probabilities, and
when the respective swap amino acid identity of the respective residue changes the identity of the respective residue, updating each corresponding residue feature set in the plurality of residue feature sets affected by the change in amino acid identity.

4. The computer system of claim 3, wherein the procedure is repeated until a convergence criterion is satisfied.

5. The computer system of claim 4, wherein the convergence criterion is a requirement that the identity of none of the amino acid residues in at least the subset of the plurality of residues is changed during the last instance of the procedure performed for each residue in at least the subset of the plurality of residues.

6. The computer system of any one of claims 3-5, wherein the obtaining a respective swap amino acid identity for the respective residue based on a draw from the corresponding plurality of probabilities comprises:
determining a respective difference, E_{final}−Eᵢₙᵢₜᵢₐₗ, between (i) a property of the polypeptide without the respective swap amino acid identity for the respective residue (E_{final}) against (ii) a property of the polypeptide with the respective swap amino acid identity for the respective residue (Eᵢₙᵢₜᵢₐₗ) to determine whether the respective swap amino acid identity for the respective residue improves the property, wherein
when the respective difference indicates that the respective swap amino acid identity for the respective residue improves the property of the polypeptide, the identity of the respective residue is changed to the respective swap amino acid identity, and
when the respective difference indicates that the respective swap amino acid identity for the respective residue fails to improve the property of the polypeptide, the identity of the respective residue is conditionally changed to the respective swap amino acid identity based on a function of the respective difference.

7. The computer system of claim 6, wherein the function of the respective difference has the form e^{-(E}^{_{final}}^{-E}^{ᵢₙᵢₜᵢₐₗ}^{)/T}, wherein T is a predetermined user adjusted temperature.

8. The computer system of claim 6 or 7, wherein the property of the polypeptide is a stability of the polypeptide in forming a heterocomplex with a polypeptide of another type.

9. The computer system of claim 8, wherein
the polypeptide is an Fc chain of a first type,
the polypeptide of another type is an Fc chain of a second type, and
the property of the polypeptide is a stability of a heterodimerization of the Fc chain of a first type with the Fc chain of the second type.

10. The computer system of claim 6 or 7,
wherein the property of the polypeptide is a composite of (i) a stability of the polypeptide within a heterocomplex with a polypeptide of another type, and (ii) a stability of the polypeptide within one or more homocomplexes.

11. The computer system of claim 6 or 7,
wherein the property of the polypeptide is a composite of (i) a combination of a stability of the polypeptide within a heterocomplex with a polypeptide of another type and a binding specificity or binding affinity of the polypeptide for the polypeptide of another type, and (ii) a combination of a stability of the polypeptide within a homocomplex and a binding specificity or binding affinity of the polypeptide for itself to form one or more homocomplexes.

12. The computer system of claim 6 or 7, wherein the property of the polypeptide is a stability of polypeptide, a pI of polypeptide, a percentage of positively charged residues in the polypeptide, an extinction coefficient of the polypeptide, an instability index of the polypeptide, or an aliphatic index of the polypeptide, or any combination thereof.

13. The computer system of any one of claims 1-12, wherein the polypeptide is an antigen-antibody complex.

14. The computer system of any one of claims 1-12, wherein the plurality of residues comprises 50 or more residues.

15. The computer system of claim 1 or any one of claims 3-14, wherein the subset of the plurality of residues is 10 or more, 20 or more, or 30 more residues within the plurality of residues.

16. The computer system of any one of claims 1-15, wherein the nearest neighbor cutoff is the K closest residues to the respective residue as determined by C_{α} carbon to C_{α} carbon distance, wherein K is a positive integer of 10 or greater.

17. The computer system of claim 16, wherein K is between 15 and 25.

18. The computer system of any one of claims 1-17, wherein the corresponding residue feature set comprises an encoding of one or more physicochemical property of each side-chain of each residue within the nearest neighbor cutoff of the C_{α} carbon of the respective residue.

19. The computer system of any one of claims 1-15, wherein the neural network comprises a first-stage one-dimensional sub-network architecture that feeds into a fully connected neural network having a final node that outputs the probability of each respective naturally occurring amino acid as a twenty element probability vector in which the twenty elements sum to 1.

20. The computer system of claim 19, wherein
the first-stage one-dimensional sub-network architecture comprises a plurality of pairs of convolutional layers, including a first pair of convolutional layers and a second pair of convolutional layers,
the first pair of convolutional layers includes a first component convolutional layer and a second component convolutional layer that each receive the residue feature set during the inputting (D),
the second pair of convolutional layers includes a third component convolutional layer and a fourth component convolutional layer,
the first component convolutional layer of the first pair of convolutional layers and the third component convolutional layer of the second pair of convolutional layers each convolve with a first filter dimension,
the second component convolutional layer of the first pair of convolutional layers and the fourth component convolutional layer of the second pair of convolutional layers each convolve with a second filter dimension that is different than the first filter dimension, and
a concatenated output of the first and second component convolutional layers of the first pair of convolutional layers serves as input to both the third component and fourth component convolutional layers of the second pair of convolutional layers.

21. The computer system of claim 20, wherein
the plurality of pairs of convolutional layers comprises between two and ten pairs of convolutional layers, and
each respective pair of convolutional layers includes a component convolutional layer that convolves with the first filter dimension,
each respective pair of convolutional layers includes a component convolutional layer that convolves with the second filter dimension, and
each respective pair of convolutional layers other than a final pair of convolutional layers in the plurality of pairs of convolutional layers passes a concatenated output of the component convolutional layers of the respective convolutional layer into each component convolutional layer of another pair of convolutional layers in the plurality of convolutional layers.

22. The computer system of claim 20 or 21 wherein the first filter dimension is one and the second filter dimension is two.

23. The computer system of any one of claims 1-19, wherein, the neural network is characterized by a first convolution filter and a second convolutional filter that are different in size.

24. The computer system of any one of claims 1-23, wherein the at least one program further comprises instructions for training the neural network to minimize a cross-entropy loss function across a training dataset of reference protein residue sites labelled by their amino acid designations obtained from a dataset of protein structures.

25. The computer system of any one of claims 1-24, wherein the at least one program further comprises
instructions for using the probability for each respective naturally occurring amino acid for the respective residue to determine an identity of the respective residue,
using the respective residue to update an atomic structure of the polypeptide, and
using the updated atomic structure of the polypeptide to determine, in silico, an interaction score between the polypeptide and a composition.

26. The computer system of claim 25, wherein
the polypeptide is an enzyme,
the composition is being screened in silico to assess an ability to inhibit an activity of the enzyme, and
the interaction score is a calculated binding coefficient of the composition to the enzyme.

27. The computer system of claim 25, wherein
the protein is a first protein,
the composition is a second protein being screened in silico to assess an ability to bind to the first protein in order to inhibit or enhance an activity of the first protein, and
the interaction score is a calculated binding coefficient of the second protein to the first protein.

28. The computer system of claim 25, wherein
the protein is a first Fc fragment of a first type,
the composition is a second Fc fragment of a second type, and
the interaction score is a calculated binding coefficient of the second Fc fragment to the first Fc fragment.

29. The computer system of any one of claims 1-28, wherein the at least one program further comprises instructions for communicating instructions to modify the polypeptide using, for the respective residue, the naturally occurring amino acid having the greatest probability in the plurality of probabilities in order to improve a stability of the polypeptide.

30. The computer system of any one of claims 1-28, wherein the at least one program further comprises instructions for communicating instructions to modify the polypeptide using, for the respective residue, the naturally occurring amino acid having the greatest probability in the plurality of probabilities in order to improve an affinity of the polypeptide for another protein.

31. The computer system of any one of claims 1-28, wherein the at least one program further comprises instructions for communicating instructions to modify the polypeptide using, for the respective residue, the naturally occurring amino acid having the greatest probability in the plurality of probabilities in order to improve a selectivity of the polypeptide in binding a second protein relative to the polypeptide binding a third protein.

32. A non-transitory computer readable storage medium storing one or more computational modules for polymer sequence prediction, the one or more computational modules collectively comprising instructions for:
(A) obtaining a plurality of atomic coordinates for at least the main chain atoms of a polypeptide, wherein the polypeptide comprises a plurality of residues;
(B) using the plurality of atomic coordinates to encode each respective residue in the plurality of residues into a corresponding residue feature set in a plurality of residue feature sets, wherein the corresponding residue feature set comprises:
for the respective residue and for each respective residue having a C_{α} carbon that is within a nearest neighbor cutoff of the C_{α} carbon of the respective residue (i) an indication of the secondary structure of the respective residue encoded as one of helix, sheet and loop, (ii) a relative solvent accessible surface area of the C_{α}, N, C, and O backbone atoms of the respective residue, and (iii) cosine and sine values of backbone dihedrals ϕ, ψ and ω of the respective residue,
a C_{α} to C_{α} distance of each neighboring residue having a C_{α} carbon within a threshold distance of the C_{α} carbon of the respective residue, and
an orientation and position of a backbone of each neighboring residue relative to the backbone residue segment of the respective residue in a reference frame centered on C_{α} atom of the respective residue;
(C) identifying a respective residue in the plurality of residues; and
(D) inputting the residue feature set corresponding to the identified respective residue into a neural network comprising at least 500 parameters thereby obtaining a plurality of probabilities, including a probability for each respective naturally occurring amino acid.

33. A method for polymer sequence prediction, the method comprising:
at a computer system comprising a memory:
A) obtaining a plurality of atomic coordinates for at least the main chain atoms of a polypeptide, wherein the polypeptide comprises a plurality of residues;
(B) using the plurality of atomic coordinates to encode each respective residue in the plurality of residues into a corresponding residue feature set in a plurality of residue feature sets, wherein the corresponding residue feature set comprises:
for the respective residue and for each respective residue having a C_{α} carbon that is within a nearest neighbor cutoff of the C_{α} carbon of the respective residue (i) an indication of the secondary structure of the respective residue encoded as one of helix, sheet and loop, (ii) a relative solvent accessible surface area of the C_{α}, N, C, and O backbone atoms of the respective residue, and (iii) cosine and sine values of backbone dihedrals ϕ, ψ and ω of the respective residue,
a C_{α} to C_{α} distance of each neighboring residue having a C_{α} carbon within a threshold distance of the C_{α} carbon of the respective residue, and
an orientation and position of a backbone of each neighboring residue relative to the backbone residue segment of the respective residue in a reference frame centered on C_{α} atom of the respective residue;
(C) identifying a respective residue in the plurality of residues; and
(D) inputting the residue feature set corresponding to the identified respective residue into a neural network comprising at least 500 parameters thereby obtaining a plurality of probabilities, including a probability for each respective naturally occurring amino acid.

34. The method of claim 33, wherein the method further comprises selecting, as the identity of the respective residue, the naturally occurring amino acid having the greatest probability in the plurality of probabilities.

35. The method of claim 33, wherein the method further comprises:
for each respective residue in at least a subset of the plurality of residues, randomly assigning an amino acid identity to the respective residue prior to the using (B),
for each respective residue in the at least a subset of the plurality of residues, performing a procedure that comprises:
performing the identifying (C) and the inputting (D) to obtain a corresponding plurality of probabilities for the respective residue,
obtaining a respective swap amino acid identity for the respective residue based on a draw from the corresponding plurality of probabilities, and
when the respective swap amino acid identity of the respective residue changes the identity of the respective residue, updating each corresponding residue feature set in the plurality of residue feature sets affected by the change in amino acid identity.

36. The method of claim 35, wherein the procedure is repeated until a convergence criterion is satisfied.

37. The method of claim 36, wherein the convergence criterion is a requirement that the identity of none of the amino acid residues in at least the subset of the plurality of residues is changed during the last instance of the procedure performed for each residue in at least the subset of the plurality of residues.

38. The method of any one of claims 35-37, wherein the obtaining a respective swap amino acid identity for the respective residue based on a draw from the corresponding plurality of probabilities comprises:
determining a respective difference, E_{final}−Eᵢₙᵢₜᵢₐₗ, between (i) a property of the polypeptide without the respective swap amino acid identity for the respective residue (E_{final}) against (ii) a property of the polypeptide with the respective swap amino acid identity for the respective residue (Eᵢₙᵢₜᵢₐₗ) to determine whether the respective swap amino acid identity for the respective residue improves the property, wherein
when the respective difference indicates that the respective swap amino acid identity for the respective residue improves the property of the polypeptide, the identity of the respective residue is changed to the respective swap amino acid identity, and
when the respective difference indicates that the respective swap amino acid identity for the respective residue fails to improve the property of the polypeptide, the identity of the respective residue is conditionally changed to the respective swap amino acid identity based on a function of the respective difference.

39. The method of claim 38, wherein the function of the respective difference has the form e^{-(E}^{_{final}}^{-E}^{ᵢₙᵢₜᵢₐₗ}^{)/T}, wherein T is a predetermined user adjusted temperature.

40. The method of claim 38 or 39, wherein the property of the polypeptide is a stability of the polypeptide in forming a heterocomplex with a polypeptide of another type.

41. The method of claim 40, wherein
the polypeptide is an Fc chain of a first type,
the polypeptide of another type is an Fc chain of a second type, and
the property of the polypeptide is a stability of a heterodimerization of the Fc chain of a first type with the Fc chain of the second type.

42. The method of claim 38 or 39,
wherein the property of the polypeptide is a composite of (i) a stability of the polypeptide within a heterocomplex with a polypeptide of another type, and (ii) a stability of the polypeptide within one or more homocomplexes.

43. The method of claim 38 or 39,
wherein the property of the polypeptide is a composite of (i) a combination of a stability of the polypeptide within a heterocomplex with a polypeptide of another type and a binding specificity or binding affinity of the polypeptide for the polypeptide of another type, and (ii) a combination of a stability of the polypeptide within a homocomplex and a binding specificity or binding affinity of the polypeptide for itself to form one or more homocomplexes.

44. The method of claim 38 or 39, wherein the property of the polypeptide is a stability of polypeptide, a pI of polypeptide, a percentage of positively charged residues in the polypeptide, an extinction coefficient of the polypeptide, an instability index of the polypeptide, or an aliphatic index of the polypeptide, or any combination thereof.

45. The method of any one of claims 33-44, wherein the polypeptide is an antigen-antibody complex.

46. The method of any one of claims 33-45, wherein the plurality of residues comprises 50 or more residues.

47. The method of claim 33 or any one of claims 35-45, wherein the subset of the plurality of residues is 10 or more, 20 or more, or 30 more residues within the plurality of residues.

48. The method of any one of claims 33-47, wherein the nearest neighbor cutoff is the K closest residues to the respective residue as determined by C carbon to C carbon distance, wherein K is a positive integer of 10 or greater.

49. The method of claim 48, wherein K is between 15 and 25.

50. The method of any one of claims 33-49, wherein the corresponding residue feature set comprises an encoding of one or more physicochemical property of each side-chain of each residue within the nearest neighbor cutoff of the C carbon of the respective residue.

51. The method of any one of claims 33-47, wherein the neural network comprises a first-stage one-dimensional sub-network architecture that feeds into a fully connected neural network having a final node that outputs the probability of each respective naturally occurring amino acid as a twenty element probability vector in which the twenty elements sum to 1.

52. The method of claim 51, wherein
the first-stage one-dimensional sub-network architecture comprises a plurality of pairs of convolutional layers, including a first pair of convolutional layers and a second pair of convolutional layers,
the first pair of convolutional layers includes a first component convolutional layer and a second component convolutional layer that each receive the residue feature set during the inputting (D),
the second pair of convolutional layers includes a third component convolutional layer and a fourth component convolutional layer,
the first component convolutional layer of the first pair of convolutional layers and the third component convolutional layer of the second pair of convolutional layers each convolve with a first filter dimension,
the second component convolutional layer of the first pair of convolutional layers and the fourth component convolutional layer of the second pair of convolutional layers each convolve with a second filter dimension that is different than the first filter dimension, and
a concatenated output of the first and second component convolutional layers of the first pair of convolutional layers serves as input to both the third component and fourth component convolutional layers of the second pair of convolutional layers.

53. The method of claim 52, wherein
the plurality of pairs of convolutional layers comprises between two and ten pairs of convolutional layers, and
each respective pair of convolutional layers includes a component convolutional layer that convolves with the first filter dimension,
each respective pair of convolutional layers includes a component convolutional layer that convolves with the second filter dimension, and
each respective pair of convolutional layers other than a final pair of convolutional layers in the plurality of pairs of convolutional layers passes a concatenated output of the component convolutional layers of the respective convolutional layer into each component convolutional layer of another pair of convolutional layers in the plurality of convolutional layers.

54. The method of claim 52 or 53 wherein the first filter dimension is one and the second filter dimension is two.

55. The method of any one of claims 33-54, wherein, the neural network is characterized by a first convolution filter and a second convolutional filter that are different in size.

56. The method of any one of claims 33-55, wherein the method further comprises training the neural network to minimize a cross-entropy loss function across a training dataset of reference protein residue sites labelled by their amino acid designations obtained from a dataset of protein structures.

57. The method of any one of claims 33-56, wherein the method further comprises
using the probability for each respective naturally occurring amino acid for the respective residue to determine an identity of the respective residue,
using the respective residue to update an atomic structure of the polypeptide, and
using the updated atomic structure of the polypeptide to determine, in silico, an interaction score between the polypeptide and a composition.

58. The method of claim 57, wherein
the polypeptide is an enzyme,
the composition is being screened in silico to assess an ability to inhibit an activity of the enzyme, and
the interaction score is a calculated binding coefficient of the composition to the enzyme.

59. The method of claim 57, wherein
the protein is a first protein,
the composition is a second protein being screened in silico to assess an ability to bind to the first protein in order to inhibit or enhance an activity of the first protein, and
the interaction score is a calculated binding coefficient of the second protein to the first protein.

60. The method of claim 57, wherein
the protein is a first Fc fragment of a first type,
the composition is a second Fc fragment of a second type, and
the interaction score is a calculated binding coefficient of the second Fc fragment to the first Fc fragment.

61. The method of any one of claims 33-60, wherein the method further comprises modifying the polypeptide using, for the respective residue, the naturally occurring amino acid having the greatest probability in the plurality of probabilities, thereby forming a modified polypeptide, in order to improve a stability of the polypeptide.

62. The method of any one of claims 33-60, wherein the method further comprises modifying the polypeptide using, for the respective residue, the naturally occurring amino acid having the greatest probability in the plurality of probabilities, thereby forming a modified polypeptide, in order to improve an affinity of the polypeptide for another protein.

63. The method of any one of claims 33-60, wherein the method further comprises modifying the polypeptide using, for the respective residue, the naturally occurring amino acid having the greatest probability in the plurality of probabilities, thereby forming a modified polypeptide, in order to improve a selectivity of the polypeptide in binding a second protein relative to the polypeptide binding a third protein.

64. The method of any one of claims 61 through 63, wherein the method further comprises using the modified polypeptide as a treatment of a medical condition associated with the polypeptide.

65. The method of claim 64, wherein the treatment comprises a composition comprising the modified polypeptide and one or more excipient and/or one or more pharmaceutically acceptable carrier and/or one or more diluent.

66. The method of claim 64 or 65, wherein the medical condition is inflammation or pain.

67. The method of claim 64 or 65, wherein the medical condition is a disease.

68. The method of claim 64 or 65, wherein the medical condition is asthma, an autoimmune disease, autoimmune lymphoproliferative syndrome (ALPS), cholera, a viral infection, Dengue fever, an E. coli infection, Eczema, hepatitis, Leprosy, Lyme Disease, Malaria, Monkeypox, Pertussis, a Yersinia pestis infection, primary immune deficiency disease, prion disease, a respiratory syncytial virus infection, Schistosomiasis, gonorrhea, genital herpes, a human papillomavirus infection, chlamydia, syphilis, Shigellosis, Smallpox, STAT3 dominant-negative disease, tuberculosis, a West Nile viral infection, or a Zika viral infection.

69. The method of any one of claims 64-68, wherein the method further comprises treating the medical condition by administering the treatment to a subject in need of treatment of the medical condition.
